SEQUENCE LISTING (104 sequences)

SEQ ID NO: 1 - protein, 323 aa, Homo sapiens
  PEPTIDE: Full-length human CTGF protein
QNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA

SEQ ID NO: 2 - protein, 348 aa, Artificial Sequence
  PEPTIDE: Synthetic CTGF-his
MEFGLSWLFLVAILKGVQCQNCSGPCRCPDEPAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMAHHHHHH

SEQ ID NO: 3 - protein, 575 aa, Artificial Sequence
  PEPTIDE: Synthetic mCTGF-Fc
MEFGLSWLFLVAILKGVQCQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMAEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 4 - protein, 348 aa, Artificial Sequence
  PEPTIDE: Synthetic Mouse CTGF protein-His tag
MEFGLSWLFLVAILKGVQCQDCSAQCQCAAEAAPHCPAGVSLVLDGCGCCRVCAKQLGELCTERDPCDPHKGLFCDFGSPANRKIGVCTAKDGAPCVFGGSVYRSGESFQSSCKYQCTCLDGAVGCVPLCSMDVRLPSPDCPFPRRVKLPGKCCEEWVCDEPKDRTAVGPALAAYRLEDTFGPDPTMMRANCLVQTTEWSACSKTCGMGISTRVTNDNTFCRLEKQSRLCMVRPCEADLEENIKKGKKCIRTPKIAKPVKFELSGCTSVKTYRAKFCGVCTDGRCCTPHRTTTLPVEFKCPDGEIMKKNMMFIKTCACHYNCPGDNDIFESLYYRKMYGDMAHHHHHH

SEQ ID NO: 5 - protein, 574 aa, Artificial Sequence
  PEPTIDE: Synthetic Cynomolgus monkey CTGF-Fc
MEFGLSWLFLVAILKGVQCQNCSGPCRCPAEQAPRCPAGVSLVLDGCGCCRVCAKQLGELCTERDPCDPHKGLFCDFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSVKTYRAKFCGVCTDGRCCTPHRTTTLPVEFKCPDGEVMKKNMMFIKTCACHYNCPGDNDIFESLYYRKMYGDMAEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 116 aa, Mus musculus
  DOMAIN: mab147 VH
EVQLVESGGGLVQPEGSLKLSCAASGFSFNTYAMNWVRQAPGKGLEWVARIRTKSNNYATYYADSVKDRFTISRDDSESMLYLQMNNLKTEDTAMYYCVETGFAYWDQGTLVTVSA

SEQ ID NO: 7 - protein, 106 aa, Mus musculus
  DOMAIN: mab147 VL
QIVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELK

SEQ ID NO: 8 - protein, 114 aa, Mus musculus
  DOMAIN: mab164 VH
QVQLKQSGPGLVQPSQSLSITCTVSGFSLTTFGVHWIRQSPGKGLEWLGVIWRRGGTDYNAAFMSRLSITKDNSRSHVFFKMTSLQTDDSAIYYCARDGGFDYWGQGTTLTVSS

SEQ ID NO: 9 - protein, 109 aa, Mus musculus
  DOMAIN: mab164 VL
QAVVTQESALTTSPGGTVILTCRSSIGAVTTSNYANWVQEKPDHLFTGLIGGTSNRAPGVPVRFSGSLIGDKAALTITGAQAEDDAMYFCALWYSTHYVFGGGTKVTVL

SEQ ID NO: 10 - protein, 5 aa, Mus musculus
  DOMAIN: mab147 HCDR1
TYAMN

SEQ ID NO: 11 - protein, 19 aa, Mus musculus
  DOMAIN: mab147 HCDR2
RIRTKSNNYATYYADSVKD

SEQ ID NO: 12 - protein, 5 aa, Mus musculus
  DOMAIN: mab147 HCDR3
TGFAY

SEQ ID NO: 13 - protein, 10 aa, Mus musculus
  DOMAIN: mab147 LCDR1
SASSSVSYMH

SEQ ID NO: 14 - protein, 7 aa, Mus musculus
  DOMAIN: mab147 LCDR2
STSNLAS

SEQ ID NO: 15 - protein, 9 aa, Mus musculus
  DOMAIN: mab147 LCDR3
QQRSSYPLT

SEQ ID NO: 16 - protein, 5 aa, Mus musculus
  DOMAIN: mab164 HCDR1
TFGVH

SEQ ID NO: 17 - protein, 16 aa, Mus musculus
  DOMAIN: mab164 HCDR2
VIWRRGGTDYNAAFMS

SEQ ID NO: 18 - protein, 6 aa, Mus musculus
  DOMAIN: mab164 HCDR3
DGGFDY

SEQ ID NO: 19 - protein, 14 aa, Mus musculus
  DOMAIN: mab164 LCDR1
RSSIGAVTTSNYAN

SEQ ID NO: 20 - protein, 7 aa, Mus musculus
  DOMAIN: mab164 LCDR2
GTSNRAP

SEQ ID NO: 21 - protein, 9 aa, Mus musculus
  DOMAIN: mab164 LCDR3
ALWYSTHYV

SEQ ID NO: 22 - protein, 106 aa, Artificial Sequence
  DOMAIN: Synthetic Hu147-VL1
EIVLTQSPATLSLSPGERATLSCSASSSVSYMHWFQQKPGQSPRLLIYSTSNLASGIPARFSGSGSGTDYTLTISSLEPEDFAVYYCQQRSSYPLTFGQGTKLEIK

SEQ ID NO: 23 - protein, 106 aa, Artificial Sequence
  DOMAIN: Synthetic Hu147-VL2
EIVLTQSPATLSLSPGERATLSCSASSSVSYMHWFQQKPGQSPKLLIYSTSNLASGIPARFSGSGSGTDYTLTISSLEPEDFAVYYCQQRSSYPLTFGQGTKLEIK

SEQ ID NO: 24 - protein, 106 aa, Artificial Sequence
  DOMAIN: Synthetic Hu147-VL3
EIVLTQSPATLSLSPGERATLSCSASSSVSYMHWFQQKPGQSPKLWIYSTSNLASGVPARFSGSGSGTDYTLTISSLEPEDFAVYYCQQRSSYPLTFGQGTKLEIK

SEQ ID NO: 25 - protein, 106 aa, Artificial Sequence
  DOMAIN: Synthetic Hu147-VL4
DIQMTQSPSSLSASVGDRVTITCSASSSVSYMHWFQQKPGKSPKLLIYSTSNLASGVPSRFSGSGSGTDYTLTISSLQPEDFATYYCQQRSSYPLTFGQGTKLEIK

SEQ ID NO: 26 - protein, 106 aa, Artificial Sequence
  DOMAIN: Synthetic Hu147-VL5
DIQLTQSPSSLSASVGDRVTITCSASSSVSYMHWFQQKPGKSPKLWIYSTSNLASGVPSRFSGSGSGTDYTLTISSLQPEDFATYYCQQRSSYPLTFGQGTKLEIK

SEQ ID NO: 27 - protein, 116 aa, Artificial Sequence
  DOMAIN: Synthetic Hu147-VH1
EVQLVESGGGLVQPGGSLRLSCAASGFTFSTYAMNWVRQAPGKGLEWVGRIRTKSNNYATYYADSVKDRFTISRDDSKNSLYLQMNSLKTEDTAVYYCVETGFAYWDQGTLVTVSS

SEQ ID NO: 28 - protein, 116 aa, Artificial Sequence
  DOMAIN: Synthetic Hu147-VH2
EVQLVESGGGLVQPGGSLRLSCAASGFTFNTYAMNWVRQAPGKGLEWVARIRTKSNNYATYYADSVKDRFTISRDDSKNSLYLQMNSLKTEDTAVYYCVETGFAYWDQGTLVTVSS

SEQ ID NO: 29 - protein, 116 aa, Artificial Sequence
  DOMAIN: Synthetic Hu147-VH3
EVQLVESGGGLVQPGGSLRLSCAASGFSFNTYAMNWVRQAPGKGLEWVARIRTKSNNYATYYADSVKDRFTISRDDSESSLYLQMNSLKTEDTAVYYCVETGFAYWDQGTLVTVSS

SEQ ID NO: 30 - protein, 109 aa, Artificial Sequence
  DOMAIN: Synthetic Hu164-VL7
ETVVTQEPSLTVSPGGTVTLTCRSSIGAVTTSNYANWVQQKPGQAFRGLIGGTSNRAPWTPARFSGSLLGGKAALTLSGVQPEDEAEYYCALWYSTHYVFGGGTKLTVL

SEQ ID NO: 31 - protein, 109 aa, Artificial Sequence
  DOMAIN: Synthetic Hu164-VL8
ETVVTQEPSFSVSPGGTVTLTCRSSIGAVTTSNYANWVQQTPGQAFRGLIGGTSNRAPGVPDRFSGSILGNKAALTITGAQADDESDYYCALWYSTHYVFGGGTKLTVL

SEQ ID NO: 32 - protein, 109 aa, Artificial Sequence
  DOMAIN: Synthetic Hu164-VL9
ESVVTQPPSVSGAPGQRVTISCRSSIGAVTTSNYANWVQQLPGTAFKGLIGGTSNRAPGVPDRFSGSKSGTSASLAITGLQAEDEADYYCALWYSTHYVFGGGTKLTVL

SEQ ID NO: 33 - protein, 114 aa, Artificial Sequence
  DOMAIN: Synthetic Hu164-VH5
EVTLKESGPVLVKPTETLTLTCTVSGFSLSTFGVHWIRQPPGKALEWLAVIWRRGGTDYNAAFMSRLTISKDTSKSQVVLTMTNMDPVDTATYYCARDGGFDYWGQGTTVTVSS

SEQ ID NO: 34 - protein, 114 aa, Artificial Sequence
  DOMAIN: Synthetic Hu164-VH6
EVTLKESGPVLVKPTETLTLTCTVSGFSLSTFGVHWIRQPPGKGLEWLGVIWRRGGTDYNAAFMSRLTISKDTSKSQVVFTMTNMDPVDTATYYCARDGGFDYWGQGTTVTVSS

SEQ ID NO: 35 - protein, 114 aa, Artificial Sequence
  DOMAIN: Synthetic Hu164-VH7
EVQLQESGPGLVKPSQTLSLTCTVSGFSISTFGVHWIRQHPGKGLEWIGVIWRRGGTDYNAAFMSRVTISKDTSKNQVSLKLSSVTAADTAVYYCARDGGFDYWGQGTTVTVSS

SEQ ID NO: 36 - protein, 114 aa, Artificial Sequence
  DOMAIN: Synthetic Hu164-VH8
EVQLQESGPGLVKPSQTLSLTCTVSGFSISTFGVHWIRQHPGKGLEWLGVIWRRGGTDYNAAFMSRLTISKDTSKNQVSFKLSSVTAADTAVYYCARDGGFDYWGQGTTVTVSS

SEQ ID NO: 37 - protein, 330 aa, Artificial Sequence [sequence omitted]

SEQ ID NO: 38 - protein, 330 aa, Artificial Sequence
  DOMAIN: Synthetic Heavy chain constant region y
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 107 aa, Artificial Sequence [sequence omitted]

SEQ ID NO: 40 - protein, 105 aa, Artificial Sequence
  DOMAIN: Synthetic Humanized antibody light chain lambda constant region
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 41 - protein, 446 aa, Artificial Sequence
  CHAIN: Synthetic Heavy chain of Ch147
EVQLVESGGGLVQPEGSLKLSCAASGFSFNTYAMNWVRQAPGKGLEWVARIRTKSNNYATYYADSVKDRFTISRDDSESMLYLQMNNLKTEDTAMYYCVETGFAYWDQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 213 aa, Artificial Sequence
  CHAIN: Synthetic Light chain of Ch147
QIVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 446 aa, Artificial Sequence
  CHAIN: Synthetic Heavy chain of Hu147-11wk
EVQLVESGGGLVQPGGSLRLSCAASGFTFSTYAMNWVRQAPGKGLEWVGRIRTKSNNYATYYADSVKDRFTISRDDSKNSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 446 aa, Artificial Sequence
  CHAIN: Synthetic Heavy chain of Hu147-21wk
EVQLVESGGGLVQPGGSLRLSCAASGFTFNTYAMNWVRQAPGKGLEWVARIRTKSNNYATYYADSVKDRFTISRDDSKNSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 446 aa, Artificial Sequence
  CHAIN: Synthetic Heavy chain of Hu147-31wk
EVQLVESGGGLVQPGGSLRLSCAASGFSFNTYAMNWVRQAPGKGLEWVARIRTKSNNYATYYADSVKDRFTISRDDSESSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 446 aa, Artificial Sequence
  CHAIN: Synthetic Heavy chain of Hu147-11yk
EVQLVESGGGLVQPGGSLRLSCAASGFTFSTYAMNWVRQAPGKGLEWVGRIRTKSNNYATYYADSVKDRFTISRDDSKNSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 446 aa, Artificial Sequence
  CHAIN: Synthetic Heavy chain of Hu147-21yk
EVQLVESGGGLVQPGGSLRLSCAASGFTFNTYAMNWVRQAPGKGLEWVARIRTKSNNYATYYADSVKDRFTISRDDSKNSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 446 aa, Artificial Sequence
  CHAIN: Synthetic Heavy chain of Hu147-31yk
EVQLVESGGGLVQPGGSLRLSCAASGFSFNTYAMNWVRQAPGKGLEWVARIRTKSNNYATYYADSVKDRFTISRDDSESSLYLQMNSLKTEDTAVYYCVETGFAYWDQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 213 aa, Artificial Sequence
  CHAIN: Synthetic Light chain of Hu147-11wk
EIVLTQSPATLSLSPGERATLSCSASSSVSYMHWFQQKPGQSPRLLIYSTSNLASGIPARFSGSGSGTDYTLTISSLEPEDFAVYYCQQRSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 213 aa, Artificial Sequence
  CHAIN: Synthetic Light chain of Hu147-12wk
EIVLTQSPATLSLSPGERATLSCSASSSVSYMHWFQQKPGQSPKLLIYSTSNLASGIPARFSGSGSGTDYTLTISSLEPEDFAVYYCQQRSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 213 aa, Artificial Sequence
  CHAIN: Synthetic Light chain of Hu147-13wk
EIVLTQSPATLSLSPGERATLSCSASSSVSYMHWFQQKPGQSPKLWIYSTSNLASGVPARFSGSGSGTDYTLTISSLEPEDFAVYYCQQRSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 213 aa, Artificial Sequence
  CHAIN: Synthetic Light chain of Hu147-14wk
DIQMTQSPSSLSASVGDRVTITCSASSSVSYMHWFQQKPGKSPKLLIYSTSNLASGVPSRFSGSGSGTDYTLTISSLQPEDFATYYCQQRSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 213 aa, Artificial Sequence
  CHAIN: Synthetic Light chain of Hu147-15wk
DIQLTQSPSSLSASVGDRVTITCSASSSVSYMHWFQQKPGKSPKLWIYSTSNLASGVPSRFSGSGSGTDYTLTISSLQPEDFATYYCQQRSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 444 aa, Artificial Sequence
  CHAIN: Synthetic Heavy chain of Ch164
QVQLKQSGPGLVQPSQSLSITCTVSGFSLTTFGVHWIRQSPGKGLEWLGVIWRRGGTDYNAAFMSRLSITKDNSRSHVFFKMTSLQTDDSAIYYCARDGGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 214 aa, Artificial Sequence
  CHAIN: Synthetic Light chain of Ch164
QAVVTQESALTTSPGGTVILTCRSSIGAVTTSNYANWVQEKPDHLFTGLIGGTSNRAPGVPVRFSGSLIGDKAALTITGAQAEDDAMYFCALWYSTHYVFGGGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 56 - protein, 444 aa, Artificial Sequence
  CHAIN: Synthetic Heavy chain of Hu164-57wl
EVTLKESGPVLVKPTETLTLTCTVSGFSLSTFGVHWIRQPPGKALEWLAVIWRRGGTDYNAAFMSRLTISKDTSKSQVVLTMTNMDPVDTATYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 444 aa, Artificial Sequence
  CHAIN: Synthetic Heavy chain of Hu164-67wl
EVTLKESGPVLVKPTETLTLTCTVSGFSLSTFGVHWIRQPPGKGLEWLGVIWRRGGTDYNAAFMSRLTISKDTSKSQVVFTMTNMDPVDTATYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 444 aa, Artificial Sequence
  CHAIN: Synthetic Heavy chain of Hu164-77wl
EVQLQESGPGLVKPSQTLSLTCTVSGFSISTFGVHWIRQHPGKGLEWIGVIWRRGGTDYNAAFMSRVTISKDTSKNQVSLKLSSVTAADTAVYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 444 aa, Artificial Sequence
  CHAIN: Synthetic Heavy chain of Hu164-87wl
EVQLQESGPGLVKPSQTLSLTCTVSGFSISTFGVHWIRQHPGKGLEWLGVIWRRGGTDYNAAFMSRLTISKDTSKNQVSFKLSSVTAADTAVYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 444 aa, Artificial Sequence
  CHAIN: Synthetic Heavy chain of Hu164-57yl
EVTLKESGPVLVKPTETLTLTCTVSGFSLSTFGVHWIRQPPGKALEWLAVIWRRGGTDYNAAFMSRLTISKDTSKSQVVLTMTNMDPVDTATYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 444 aa, Artificial Sequence
  CHAIN: Synthetic Heavy chain of Hu164-67yl
EVTLKESGPVLVKPTETLTLTCTVSGFSLSTFGVHWIRQPPGKGLEWLGVIWRRGGTDYNAAFMSRLTISKDTSKSQVVFTMTNMDPVDTATYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 444 aa, Artificial Sequence
  CHAIN: Synthetic Heavy chain of Hu164-77yl
EVQLQESGPGLVKPSQTLSLTCTVSGFSISTFGVHWIRQHPGKGLEWIGVIWRRGGTDYNAAFMSRVTISKDTSKNQVSLKLSSVTAADTAVYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 444 aa, Artificial Sequence
  CHAIN: Synthetic Heavy chain of Hu164-87yl
EVQLQESGPGLVKPSQTLSLTCTVSGFSISTFGVHWIRQHPGKGLEWLGVIWRRGGTDYNAAFMSRLTISKDTSKNQVSFKLSSVTAADTAVYYCARDGGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 214 aa, Artificial Sequence
  CHAIN: Synthetic Light chain of Hu164-57wl
ETVVTQEPSLTVSPGGTVTLTCRSSIGAVTTSNYANWVQQKPGQAFRGLIGGTSNRAPWTPARFSGSLLGGKAALTLSGVQPEDEAEYYCALWYSTHYVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 65 - protein, 214 aa, Artificial Sequence
  CHAIN: Synthetic Light chain of Hu164-58wl
ETVVTQEPSFSVSPGGTVTLTCRSSIGAVTTSNYANWVQQTPGQAFRGLIGGTSNRAPGVPDRFSGSILGNKAALTITGAQADDESDYYCALWYSTHYVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 66 - protein, 214 aa, Artificial Sequence
  CHAIN: Synthetic Light chain of Hu164-59wl
ESVVTQPPSVSGAPGQRVTISCRSSIGAVTTSNYANWVQQLPGTAFKGLIGGTSNRAPGVPDRFSGSKSGTSASLAITGLQAEDEADYYCALWYSTHYVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 67 - protein, 450 aa, Artificial Sequence
  CHAIN: Synthetic Heavy chain of mAb1
EGQLVQSGGGLVHPGGSLRLSCAGSGFTFSSYGMHWVRQAPGKGLEWVSGIGTGGGTYSTDSVKGRFTISRDNAKNSLYLQMNSLRAEDMAVYYCARGDYYGSGSFFDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 214 aa, Artificial Sequence
  CHAIN: Synthetic Light chain of mAb1
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 122 aa, Artificial Sequence
  CHAIN: Synthetic mab95 VH
EVLLQQSGPVLVKPGPSVKISCKASGFTFTNYDIHWVKQSHGKSLEWIGLVYPYNGGTAYNQKFKDKATLTFDTSSSTAYMELNSLTSEDSAVYYCARWGLIPGTTSYFDVWGTGTTVTVSS

SEQ ID NO: 70 - protein, 106 aa, Artificial Sequence
  DOMAIN: Synthetic mab95 VL
QIVLSQSPPILSASPGEKVTMTCRASSSVSYIHWYQQKPRSSPKPWIYATSNLASGVPARFSGSGSGTSYSLTISRVEAEDAATYYCQQWNSNPWTFGGGTRLEIK

SEQ ID NO: 71 - protein, 5 aa, Artificial Sequence
  DOMAIN: Synthetic mab95 HCDR1
NYDIH

SEQ ID NO: 72 - protein, 17 aa, Artificial Sequence
  DOMAIN: Synthetic mab95 HCDR2
LVYPYNGGTAYNQKFKD

SEQ ID NO: 73 - protein, 13 aa, Artificial Sequence
  DOMAIN: Synthetic mab95 HCDR3
WGLIPGTTSYFDV

SEQ ID NO: 74 - protein, 10 aa, Artificial Sequence
  DOMAIN: Synthetic mab95 LCDR1
RASSSVSYIH

SEQ ID NO: 75 - protein, 7 aa, Artificial Sequence
  DOMAIN: Synthetic mab95 LCDR2
ATSNLAS

SEQ ID NO: 76 - protein, 9 aa, Artificial Sequence
  DOMAIN: Synthetic mab95 LCDR3
QQWNSNPWT

SEQ ID NO: 77 - protein, 106 aa, Artificial Sequence
  CHAIN: Synthetic Hu95-VL1
EIVLTQSPATLSLSPGERATLSCRASSSVSYIHWYQQKPGQAPRPWIYATSNLASGIPARFSGSGSGTDYTLTISRLEPEDFAVYYCQQWNSNPWTFGGGTKVEIK

SEQ ID NO: 78 - protein, 106 aa, Artificial Sequence
  CHAIN: Synthetic Hu95-VL2
EIVLTQSPATLSLSPGERATLSCRASSSVSYIHWYQQKPGQSPRPWIYATSNLASGVPARFSGSGSGTSYTLTISRLEPEDFAVYYCQQWNSNPWTFGGGTKVEIK

SEQ ID NO: 79 - protein, 105 aa, Artificial Sequence
  CHAIN: Synthetic Hu95-VL3
ESVLTQPPSVSGAPGQRVTISCRASSSVSYIHWYQQLPGTAPKPWIYATSNLASGVPDRFSGSKSGTSYSLAITGLQAEDEADYYCQQWNSNPWTFGGGTKVEIK

SEQ ID NO: 80 - protein, 105 aa, Artificial Sequence
  CHAIN: Synthetic Hu95-VL4
EIVLTQPPSVSGAPGQRVTISCRASSSVSYIHWYQQLPGTSPKPWIYATSNLASGVPDRFSGSKSGTSYSLAITGLQAEDEADYYCQQWNSNPWTFGGGTKVEIK

SEQ ID NO: 81 - protein, 122 aa, Artificial Sequence
  CHAIN: Synthetic Hu95-VH1
EVQLVQSGAEVKKPGASVKVSCKASGFTFTNYDIHWVRQAPGQRLEWMGLVYPYNGGTAYNQKFKDRVTLTFDTSASTAYMELSSLRSEDTAVYYCARWGLIPGTTSYFDVWGQGTTVTVSS

SEQ ID NO: 82 - protein, 122 aa, Artificial Sequence
  CHAIN: Synthetic Hu95-VH2
EVQLVQSGAEVKKPGASVKVSCKASGFTFTNYDIHWVRQAPGQRLEWIGLVYPYNGGTAYNQKFKDRATLTFDTSASTAYMELSSLRSEDTAVYYCARWGLIPGTTSYFDVWGQGTTVTVSS

SEQ ID NO: 83 - protein, 122 aa, Artificial Sequence
  CHAIN: Synthetic Hu95-VH3
EVQLVQSGAEVKKPGASVKVSCKASGFTFTNYDIHWVKQAPGQRLEWMGLVYPYNGGTAYNQKFKDKVTLTFDTSASTAYMELSSLRSEDTAVYYCARWGLIPGTTSYFDVWGQGTTVTVSS

SEQ ID NO: 84 - protein, 122 aa, Artificial Sequence
  CHAIN: Synthetic Hu95-VH4
EVQLVQSGAEVKKPGASVKVSCKASGFTFTNYDIHWVKQAPGQRLEWIGLVYPYNGGTAYNQKFKDKATLTFDTSASTAYMELSSLRSEDTAVYYCARWGLIPGTTSYFDVWGQGTTVTVSS

SEQ ID NO: 85 - protein, 122 aa, Artificial Sequence
  CHAIN: Synthetic Hu95-VH5
EVQLVQSGAEVKKPGASVKVSCRASGFTFTNYAIHWVKQAPGQRLEWMGLVYPYTGGTAYNQKFKDKVTLTFDTSASTAYMELSSLRSEDTAVYYCARWGMIPGTNSYFDVWGQGTTVTVSS

SEQ ID NO: 86 - protein, 452 aa, Artificial Sequence
  CHAIN: Synthetic Heavy chain of Ch95
EVLLQQSGPVLVKPGPSVKISCKASGFTFTNYDIHWVKQSHGKSLEWIGLVYPYNGGTAYNQKFKDKATLTFDTSSSTAYMELNSLTSEDSAVYYCARWGLIPGTTSYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 213 aa, Artificial Sequence
  CHAIN: Synthetic Light chain of Ch95
QIVLSQSPPILSASPGEKVTMTCRASSSVSYIHWYQQKPRSSPKPWIYATSNLASGVPARFSGSGSGTSYSLTISRVEAEDAATYYCQQWNSNPWTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 452 aa, Artificial Sequence
  CHAIN: Synthetic Heavy chain of Hu95-11wk
EVQLVQSGAEVKKPGASVKVSCKASGFTFTNYDIHWVRQAPGQRLEWMGLVYPYNGGTAYNQKFKDRVTLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 452 aa, Artificial Sequence
  CHAIN: Synthetic Heavy chain of Hu95-21wk
EVQLVQSGAEVKKPGASVKVSCKASGFTFTNYDIHWVRQAPGQRLEWIGLVYPYNGGTAYNQKFKDRATLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 452 aa, Artificial Sequence
  CHAIN: Synthetic Heavy chain of Hu95-31wk
EVQLVQSGAEVKKPGASVKVSCKASGFTFTNYDIHWVKQAPGQRLEWMGLVYPYNGGTAYNQKFKDKVTLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 452 aa, Artificial Sequence
  CHAIN: Synthetic Heavy chain of Hu95-41wk
EVQLVQSGAEVKKPGASVKVSCKASGFTFTNYDIHWVKQAPGQRLEWIGLVYPYNGGTAYNQKFKDKATLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 452 aa, Artificial Sequence
  CHAIN: Synthetic Heavy chain of Hu95-51wk
EVQLVQSGAEVKKPGASVKVSCRASGFTFTNYAIHWVKQAPGQRLEWMGLVYPYTGGTAYNQKFKDKVTLTFDTSASTAYMELSSLRSEDTAVYYCARWGMIPGTNSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 452 aa, Artificial Sequence
  CHAIN: Synthetic Heavy chain of Hu95-11yk
EVQLVQSGAEVKKPGASVKVSCKASGFTFTNYDIHWVRQAPGQRLEWMGLVYPYNGGTAYNQKFKDRVTLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 452 aa, Artificial Sequence
  CHAIN: Synthetic Heavy chain of Hu95-21yk
EVQLVQSGAEVKKPGASVKVSCKASGFTFTNYDIHWVRQAPGQRLEWIGLVYPYNGGTAYNQKFKDRATLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 452 aa, Artificial Sequence
  CHAIN: Synthetic Hu95-31yk
EVQLVQSGAEVKKPGASVKVSCKASGFTFTNYDIHWVKQAPGQRLEWMGLVYPYNGGTAYNQKFKDKVTLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 452 aa, Artificial Sequence
  CHAIN: Synthetic Heavy chain of Hu95-41yk
EVQLVQSGAEVKKPGASVKVSCKASGFTFTNYDIHWVKQAPGQRLEWIGLVYPYNGGTAYNQKFKDKATLTFDTSASTAYMELSSLRSEDTAVYYCARWGLIPGTTSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 452 aa, Artificial Sequence
  CHAIN: Synthetic Heavy chain of Hu95-51yk
EVQLVQSGAEVKKPGASVKVSCRASGFTFTNYAIHWVKQAPGQRLEWMGLVYPYTGGTAYNQKFKDKVTLTFDTSASTAYMELSSLRSEDTAVYYCARWGMIPGTNSYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 213 aa, Artificial Sequence
  CHAIN: Synthetic Light chain of Hu95-11wk
EIVLTQSPATLSLSPGERATLSCRASSSVSYIHWYQQKPGQAPRPWIYATSNLASGIPARFSGSGSGTDYTLTISRLEPEDFAVYYCQQWNSNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 213 aa, Artificial Sequence
  CHAIN: Synthetic Light chain of Hu95-12wk
EIVLTQSPATLSLSPGERATLSCRASSSVSYIHWYQQKPGQSPRPWIYATSNLASGVPARFSGSGSGTSYTLTISRLEPEDFAVYYCQQWNSNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 212 aa, Artificial Sequence
  CHAIN: Synthetic Light chain of Hu95-13wk
ESVLTQPPSVSGAPGQRVTISCRASSSVSYIHWYQQLPGTAPKPWIYATSNLASGVPDRFSGSKSGTSYSLAITGLQAEDEADYYCQQWNSNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 212 aa, Artificial Sequence
  CHAIN: Synthetic Light chain of Hu95-14wk
EIVLTQPPSVSGAPGQRVTISCRASSSVSYIHWYQQLPGTSPKPWIYATSNLASGVPDRFSGSKSGTSYSLAITGLQAEDEADYYCQQWNSNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 5 aa, Artificial Sequence
  DOMAIN: Synthetic Hu95-VH5 HCDR1
NYAIH

SEQ ID NO: 103 - protein, 17 aa, Artificial Sequence
  DOMAIN: Synthetic Hu95-VH5 HCDR2
LVYPYTGGTAYNQKFKD

SEQ ID NO: 104 - protein, 13 aa, Artificial Sequence
  DOMAIN: Synthetic Hu95-VH5 HCDR3
WGMIPGTNSYFDV

(19) United States
(12) Patent Application Publication
(10) Pub. No.: US 2023/0416352 A1
(43) Pub. Date: Dec. 28, 2023

(54) PHARMACEUTICAL COMPOSITION COMPRISING ANTI-CONNECTIVE TISSUE GROWTH FACTOR ANTIBODY

(71) Applicants: JIANGSU HENGRUI PHARMACEUTICALS CO., LTD., Lianyungang, Jiangsu (CN); SHANGHAI HENGRUI PHARMACEUTICAL CO., LTD., Shanghai (CN)

(72) Inventors: Xiazhen MA, Shanghai (CN); Tingting WU, Shanghai (CN); Xun LIU, Shanghai (CN)

(73) Assignees: JIANGSU HENGRUI PHARMACEUTICALS CO., LTD., Lianyungang, Jiangsu (CN); SHANGHAI HENGRUI PHARMACEUTICAL CO., LTD., Shanghai (CN)

(21) Appl. No.: 18/039,683

(22) PCT Filed: Dec. 3, 2021
(86) PCT No.: PCT/CN2021/135203

§ 371 Date: May 31, 2023

(30) Foreign Application Priority Data
Dec. 3, 2020 (CN) 202011405490.5
Nov. 18, 2021 (CN) 202111366667.X

(51) Int. Cl. C07K 16/22; A61P 19/00

(52) U.S. Cl. CPC C07K 16/22; A61P 19/00; C07K 2317/565

(57) ABSTRACT

Provided is a pharmaceutical composition comprising an anti-connective tissue growth factor antibody. In another aspect, further provided is a use of the pharmaceutical composition comprising an anti-connective tissue growth factor antibody.

### TECHNICAL FIELD

The present disclosure belongs to the field of pharmaceutical formulations and particularly relates to a pharmaceutical composition comprising an antibody and use thereof as a medicament.

### BACKGROUND

The statements herein merely provide background information related to the present disclosure and may not necessarily constitute the prior art.

Expression of connective tissue growth factor (CTGF) is induced by members of the transforming growth factor β (TGFβ) superfamily. The superfamily includes TGFβ-1, -2, and -3, bone morphogenetic protein (BMP)-2, and activin. Many modulators including dexamethasone, thrombin, vascular endothelial growth factor (VEGF), and angiotensin II, and environmental stimuli including hyperglycemia and hypertension also induce CTGF expression.

TGFβ stimulation of CTGF expression is rapid and prolonged and does not require persistent application of TGFβ. TGFβ activates transcription via the DNA regulatory elements in the CTGF promoter, leading to enhanced CTGF expression. CTGF expression is up-regulated in glomerulonephritis, IgA nephropathy, focal and segmental glomerulosclerosis and diabetic nephropathy. An increase in the number of cells expressing CTGF is also observed at sites of chronic tubulointerstitial damage, and CTGF levels correlate with the degree of damage. Further, CTGF expression is increased in the glomeruli and tubulointerstitium in a variety of renal diseases associated with scarring and sclerosis of renal parenchyma. Elevated levels of CTGF are also associated with liver fibrosis, myocardial infarction, and pulmonary fibrosis. For example, in patients with idiopathic pulmonary fibrosis (IPF), CTGF is strongly up-regulated in biopsies and bronchoalveolar lavage fluid cells. Thus, CTGF is a valid therapeutic target in the diseases described above.

### SUMMARY

The present disclosure provides a pharmaceutical composition comprising an anti-CTGF antibody. The composition has the advantages of having good stability, good lyophilization morphology, etc.

In some embodiments, the pharmaceutical composition according to any one of the above is provided, wherein the anti-CTGF antibody comprises a heavy chain variable region and a light chain variable region, wherein:
i) the heavy chain variable region comprises HCDR1, HCDR2, and HCDR3 having identical sequences to those of a heavy chain variable region set forth in SEQ ID NO: 6, and the light chain variable region comprises LCDR1, LCDR2, and LCDR3 having identical sequences to those of a light chain variable region set forth in SEQ ID NO: 7; or
ii) the heavy chain variable region comprises HCDR1, HCDR2, and HCDR3 having identical sequences to those of a heavy chain variable region set forth in SEQ ID NO: 8, and the light chain variable region comprises LCDR1, LCDR2, and LCDR3 having identical sequences to those of a light chain variable region set forth in SEQ ID NO: 9.

In some embodiments, the pharmaceutical composition according to any one of the above is provided, wherein the anti-CTGF antibody comprises a heavy chain variable region and a light chain variable region, wherein:
ii-i) the heavy chain variable region comprises HCDR1, HCDR2, and HCDR3 having identical sequences to those of a heavy chain variable region set forth in SEQ ID NO: 69, and the light chain variable region comprises LCDR1, LCDR2, and LCDR3 having identical sequences to those of a light chain variable region set forth in SEQ ID NO: 70; or
ii-ii) the heavy chain variable region comprises HCDR1, HCDR2, and HCDR3 having identical sequences to those of a heavy chain variable region set forth in SEQ ID NO: 85, and the light chain variable region comprises LCDR1, LCDR2, and LCDR3 having identical sequences to those of a light chain variable region set forth in SEQ ID NO: 70.

In some embodiments, the pharmaceutical composition according to any one of the above is provided, wherein the anti-CTGF antibody comprises a heavy chain variable region and a light chain variable region, wherein:
iii) the heavy chain variable region comprises HCDR1, HCDR2, and HCDR3 set forth in SEQ ID NO: 10, SEQ ID NO: 11, and SEQ ID NO: 12, respectively, and the light chain variable region comprises LCDR1, LCDR2, and LCDR3 set forth in SEQ ID NO: 13, SEQ ID NO: 14, and SEQ ID NO: 15, respectively; or
iv) the heavy chain variable region comprises HCDR1, HCDR2, and HCDR3 set forth in SEQ ID NO: 16, SEQ ID NO: 17, and SEQ ID NO: 18, respectively, and the light chain variable region comprises LCDR1, LCDR2, and LCDR3 set forth in SEQ ID NO: 19, SEQ ID NO: 20, and SEQ ID NO: 21, respectively.

In some embodiments, the pharmaceutical composition according to any one of the above is provided, wherein the anti-CTGF antibody comprises a heavy chain variable region and a light chain variable region, wherein:
iv-i) the heavy chain variable region comprises HCDR1, HCDR2, and HCDR3 set forth in SEQ ID NO: 71, SEQ ID NO: 72, and SEQ ID NO: 73, respectively, and the light chain variable region comprises LCDR1, LCDR2, and LCDR3 set forth in SEQ ID NO: 74, SEQ ID NO: 75, and SEQ ID NO: 76, respectively; or
iv-ii) the heavy chain variable region comprises HCDR1, HCDR2, and HCDR3 set forth in SEQ ID NO: 102, SEQ ID NO: 103, and SEQ ID NO: 104, respectively, and the light chain variable region comprises LCDR1, LCDR2, and LCDR3 set forth in SEQ ID NO: 74, SEQ ID NO: 75, and SEQ ID NO: 76, respectively.

In some embodiments, the anti-CTGF antibody according to any one of the above is a murine antibody, a chimeric antibody, or a humanized antibody.

In some embodiments, the pharmaceutical composition according to any one of the above is provided, wherein the anti-CTGF antibody comprises a heavy chain variable region and a light chain variable region, wherein:
(v-1) the heavy chain variable region's amino acid sequence has at least 90% sequence identity to SEQ ID NO: 6, 27, 28, or 29, and the light chain variable region's amino acid sequence has at least 90% sequence identity to SEQ ID NO: 7, 22, 23, 24, 25, or 26;
(vi-1) the heavy chain variable region's amino acid sequence has at least 90% sequence identity to SEQ ID NO: 8, 33, 34, 35, or 36, and the light chain variable region's amino acid sequence has at least 90% sequence identity to SEQ ID NO: 9, 30, 31, or 32; or
(vi-i) the heavy chain variable region's amino acid sequence has at least 90% sequence identity to SEQ ID NO: 69, 81, 82, 83, 84, or 85, and the light chain variable region's amino acid sequence has at least 90% sequence identity to SEQ ID NO: 70, 77, 78, 79, or 80.

In some embodiments, the pharmaceutical composition according to any one of the above is provided, wherein the anti-CTGF antibody comprises a heavy chain variable region and a light chain variable region, wherein:
(v) the heavy chain variable region has at least 90%, 91%, 92%, 93%, 94%, 95%, 96%, 97%, 98%, 99%, or 100% identity to a heavy chain variable region set forth in SEQ ID NO: 6, 27, 28, or 29, and the light chain variable region has at least 90%, 91%, 92%, 93%, 94%, 95%, 96%, 97%, 98%, 99%, or 100% identity to a light chain variable region set forth in SEQ ID NO: 7, 22, 23, 24, 25, or 26; or
(vi) the heavy chain variable region has at least 90%, 91%, 92%, 93%, 94%, 95%, 96%, 97%, 98%, 99%, or 100% identity to a heavy chain variable region set forth in SEQ ID NO: 8, 33, 34, 35, or 36, and the light chain variable region has at least 90%, 91%, 92%, 93%, 94%, 95%, 96%, 97%, 98%, 99%, or 100% identity to a light chain variable region set forth in SEQ ID NO: 9, 30, 31, or 32; or
the heavy chain variable region has at least 90%, 91%, 92%, 93%, 94%, 95%, 96%, 97%, 98%, 99%, or 100% identity to the heavy chain variable region set forth in SEQ ID NO: 6, and the light chain variable region has at least 90%, 91%, 92%, 93%, 94%, 95%, 96%, 97%, 98%, 99%, or 100% identity to the light chain variable region set forth in SEQ ID NO: 7;
the heavy chain variable region has at least 90%, 91%, 92%, 93%, 94%, 95%, 96%, 97%, 98%, 99%, or 100% identity to the heavy chain variable region set forth in SEQ ID NO: 27, 28, or 29, and the light chain variable region has at least 90%, 91%, 92%, 93%, 94%, 95%, 96%, 97%, 98%, 99%, or 100% identity to the light chain variable region set forth in SEQ ID NO: 22, 23, 24, 25, or 26;
the heavy chain variable region has at least 90%, 91%, 92%, 93%, 94%, 95%, 96%, 97%, 98%, 99%, or 100% identity to the heavy chain variable region set forth in SEQ ID NO: 8, and the light chain variable region has at least 90%, 91%, 92%, 93%, 94%, 95%, 96%, 97%, 98%, 99%, or 100% identity to the light chain variable region set forth in SEQ ID NO: 9;
the heavy chain variable region has at least 90%, 91%, 92%, 93%, 94%, 95%, 96%, 97%, 98%, 99%, or 100% identity to the heavy chain variable region set forth in SEQ ID NO: 33, 34, 35, or 36, and the light chain variable region has at least 90%, 91%, 92%, 93%, 94%, 95%, 96%, 97%, 98%, 99%, or 100% identity to the light chain variable region set forth in SEQ ID NO: 30, 31, or 32.

In some embodiments, the pharmaceutical composition according to any one of the above is provided, wherein the anti-CTGF antibody comprises a heavy chain variable region and a light chain variable region, wherein:
(vi-ii) the heavy chain variable region has at least 90%, 91%, 92%, 93%, 94%, 95%, 96%, 97%, 98%, 99%, or 100% identity to a heavy chain variable region set forth in SEQ ID NO: 69, 81, 82, 83, 84, or 85, and the light chain variable region has at least 90%, 91%, 92%, 93%, 94%, 95%, 96%, 97%, 98%, 99%, or 100% identity to a light chain variable region set forth in SEQ ID NO: 70, 77, 78, 79, or 80.

In some embodiments, the pharmaceutical composition according to any one of the above is provided, wherein the anti-CTGF antibody is a humanized antibody comprising a framework region of a human antibody or a framework region variant thereof, and the framework region variant has at most 10 back mutations on a light chain framework region and/or a heavy chain framework region of the human antibody.

In some embodiments, the pharmaceutical composition according to any one of the above is provided, wherein the anti-CTGF antibody comprises a heavy chain variable region and a light chain variable region as described below:
(a) the light chain variable region comprising LCDR1, LCDR2, and LCDR3 set forth in SEQ ID NO: 13, SEQ ID NO: 14, and SEQ ID NO: 15, respectively, and comprising one or more amino acid back mutations selected from the group consisting of 4L, 36F, 43S, 45K, 47W, 58V, and 71Y, and/or the heavy chain variable region comprising HCDR1, HCDR2, and HCDR3 set forth in SEQ ID NO: 10, SEQ ID NO: 11, and SEQ ID NO: 12, respectively, and comprising one or more amino acid back mutations selected from the group consisting of 28S, 30N, 49A, 75E, 76S, 93V, 94E, and 104D; or
(b) the light chain variable region comprising LCDR1, LCDR2, and LCDR3 set forth in SEQ ID NO: 19, SEQ ID NO: 20, and SEQ ID NO: 21, respectively, and comprising one or more back mutations selected from the group consisting of 36V, 44F, 46G, and 49G, and/or the heavy chain variable region comprising HCDR1, HCDR2, and HCDR3 set forth in SEQ ID NO: 16, SEQ ID NO: 17, and SEQ ID NO: 18, respectively, and comprising one or more back mutations selected from the group consisting of 44G, 49G, 27F, 48L, 67L, 71K, 78V, and 80F.

In some embodiments, the pharmaceutical composition according to any one of the above is provided, wherein the anti-CTGF antibody comprises a heavy chain variable region and a light chain variable region as described below:
(b-i) the light chain variable region comprising LCDR1, LCDR2, and LCDR3 set forth in SEQ ID NO: 74, SEQ ID NO: 75, and SEQ ID NO: 76, respectively, and comprising one or more back mutations selected from the group consisting of 45P, 46W, 48Y, 69S, and 70Y, and the heavy chain variable region comprising HCDR1, HCDR2, and HCDR3 set forth in SEQ ID NO: 71, SEQ ID NO: 72, and SEQ ID NO: 73, respectively, and comprising one or more of back mutations 27F, 38K, 481, 67K, 68A, 70L, and 72F; or
(b-ii) the light chain variable region comprising LCDR1, LCDR2, and LCDR3 set forth in SEQ ID NO: 74, SEQ ID NO: 75, and SEQ ID NO: 76, respectively, and comprising one or more back mutations selected from the group consisting of 45P, 46W, 48Y, 69S, and 70Y, and the heavy chain variable region comprising HCDR1, HCDR2, and HCDR3 set forth in SEQ ID NO: 102, SEQ ID NO: 103, and SEQ ID NO: 104, respectively, and comprising one or more of back mutations 27F, 38K, 481, 67K, 68A, 70L, and 72F.

In some embodiments, the pharmaceutical composition according to any one of the above is provided, wherein the anti-CTGF antibody comprises a heavy chain variable region and a light chain variable region as shown below:
(vii) the heavy chain variable region having a sequence set forth in SEQ ID NO: 6 and the light chain variable region having a sequence set forth in SEQ ID NO: 7;
(viii) the heavy chain variable region having a sequence set forth in SEQ ID NO: 27, 28, or 29 and the light chain variable region having a sequence set forth in SEQ ID NO: 22, 23, 24, 25, or 26;
(ix) the heavy chain variable region having a sequence set forth in SEQ ID NO: 8 and the light chain variable region having a sequence set forth in SEQ ID NO: 9; or
(x) the heavy chain variable region having a sequence set forth in SEQ ID NO: 33, 34, 35, or 36 and the light chain variable region having a sequence set forth in SEQ ID NO: 30, 31, or 32.

In some embodiments, the pharmaceutical composition according to any one of the above is provided, wherein the anti-CTGF antibody comprises a heavy chain variable region and a light chain variable region as shown below:
(xi) the heavy chain variable region having an amino acid sequence set forth in SEQ ID NO: 69, and the light chain variable region having an amino acid sequence set forth in SEQ ID NO: 70; or
(xii) the heavy chain variable region having an amino acid sequence set forth in SEQ ID NO: 81, 82, 83, 84, or 85, and the light chain variable region having an amino acid sequence set forth in SEQ ID NO: 77, 78, 79, or 80.

In some embodiments, the pharmaceutical composition according to any one of the above is provided, wherein the anti-CTGF antibody comprises a heavy chain variable region and a light chain variable region as shown in Table 1, Table 2, and Table 3 below:

TABLE 1
Light and heavy chain variable region combinations
of a humanized antibody derived from mab 147

| | Hu147- | Hu147- | Hu147- | Hu147- | Hu147- |
|---|---|---|---|---|---|
| | VL1 | VL2 | VL3 | VL4 | VL5 |
| | SEQ ID | SEQ ID | SEQ ID | SEQ ID | SEQ ID |
| | NO: 22 | NO: 23 | NO: 24 | NO: 25 | NO: 26 |
| Hu147-VH1 | Hu147-11 | Hu147-12 | Hu147-13 | Hu147-14 | Hu147-15 |
| SEQ ID NO: | | | | | |
| 27 | | | | | |
| Hu147-VH2 | Hu147-21 | Hu147-22 | Hu147-23 | Hu147-24 | Hu147-25 |
| SEQ ID NO: | | | | | |
| 28 | | | | | |
| Hu147-VH3 | Hu147-31 | Hu147-32 | Hu147-33 | Hu147-34 | Hu147-35 |
| SEQ ID NO: | | | | | |
| 29 | | | | | |

TABLE 2
Light and heavy chain variable region combinations
of a humanized antibody of mab 164

| | Hu164- | Hu164- | Hu164- | Hu164- |
|---|---|---|---|---|
| | VH5 | VH6 | VH7 | VH8 |
| | SEQ ID | SEQ ID | SEQ ID | SEQ ID |
| | NO: 33 | NO: 34 | NO: 35 | NO: 36 |
| Hu164-VL7 | Hu164-57 | Hu164-67 | Hu164-77 | Hu164-87 |
| SEQ ID NO: 30 | | | | |
| Hu164-VL8 | Hu164-58 | Hu164-68 | Hu164-78 | Hu164-88 |
| SEQ ID NO: 31 | | | | |
| Hu164-VL9 | Hu164-59 | Hu164-69 | Hu164-79 | Hu164-89 |
| SEQ ID NO: 32 | | | | |

TABLE 3
Light and heavy chain variable region combinations
of a humanized antibody of mab95

| | Hu95- | Hu95- | Hu95- | Hu95- | Hu95- |
|---|---|---|---|---|---|
| | VH1 | VH2 | VH3 | VH4 | VH5 |
| | SEQ ID | SEQ ID | SEQ ID | SEQ ID | SEQ ID |
| | NO: 81 | NO: 82 | NO: 83 | NO: 84 | NO: 85 |
| Hu95-VL1 | Hu95-11 | Hu95-21 | Hu95-31 | Hu95-41 | Hu95-51 |
| SEQ ID NO: | | | | | |
| 77 | | | | | |
| Hu95-VL2 | Hu95-12 | Hu95-22 | Hu95-32 | Hu95-42 | Hu95-52 |
| SEQ ID NO: | | | | | |
| 78 | | | | | |
| Hu95-VL3 | Hu95-13 | Hu95-23 | Hu95-33 | Hu95-43 | Hu95-53 |
| SEQ ID NO: | | | | | |
| 79 | | | | | |
| Hu95-VL4 | Hu95-14 | Hu95-24 | Hu95-34 | Hu95-44 | Hu95-54 |
| SEQ ID NO: | | | | | |
| 80 | | | | | |

In some embodiments, the pharmaceutical composition according to any one of the above is provided, wherein the anti-CTGF antibody comprises a heavy chain variable region and a light chain variable region as shown below:
(xiii) the heavy chain variable region having an amino acid sequence set forth in SEQ ID NO: 27, and the light chain variable region having an amino acid sequence set forth in SEQ ID NO: 22;
(xiv) the heavy chain variable region having an amino acid sequence set forth in SEQ ID NO: 34, and the light chain variable region having an amino acid sequence set forth in SEQ ID NO: 30; or
(xv) the heavy chain variable region having an amino acid sequence set forth in SEQ ID NO: 85, and the light chain variable region having an amino acid sequence set forth in SEQ ID NO: 77.

In some embodiments, the pharmaceutical composition according to any one of the above is provided, wherein the anti-CTGF antibody further comprises an antibody heavy chain constant region and an antibody light chain constant region; preferably, the heavy chain constant region is selected from the group consisting of constant regions of human IgG1, IgG2, IgG3 and IgG4 and conventional variants thereof, and the light chain constant region is selected from the group consisting of constant regions of human antibody κ and λ chains and conventional variants thereof; more preferably, the antibody comprises a heavy chain constant region set forth in SEQ ID NO: 37 or 38 and a light chain constant region set forth in SEQ ID NO: 39 or 40.

In some embodiments, the pharmaceutical composition according to any one of the above is provided, wherein the anti-CTGF antibody comprises:
(c) a heavy chain having at least 85%, 90%, 91%, 92%, 93%, 94%, 95%, 96%, 97%, 98%, 99%, or 100% identity to a heavy chain having a sequence set forth in SEQ ID NO: 41, 43, 44, 45, 46, 47, or 48 and a light chain having at least 85%, 90%, 91%, 92%, 93%, 94%, 95%, 96%, 97%, 98%, 99%, or 100% identity to a light chain having a sequence set forth in SEQ ID NO: 42, 49, 50, 51, 52, or 53;
(d) a heavy chain having at least 85%, 90%, 91%, 92%, 93%, 94%, 95%, 96%, 97%, 98%, 99%, or 100% identity to a heavy chain having a sequence set forth in SEQ ID NO: 54, 56, 57, 58, 59, 60, 61, 62, or 63 and a light chain having at least 85%, 90%, 91%, 92%, 93%, 94%, 95%, 96%, 97%, 98%, 99%, or 100% identity to a light chain having a sequence set forth in SEQ ID NO: 55, 64, 65, or 66;
(e) a heavy chain having a sequence set forth in SEQ ID NO: 41, 43, 44, 45, 46, 47, or 48 and a light chain having a sequence set forth in SEQ ID NO: 42, 49, 50, 51, 52, or 53; or
(f) a heavy chain having a sequence set forth in SEQ ID NO: 54, 56, 57, 58, 59, 60, 61, 62, or 63 and a light chain having a sequence set forth in SEQ ID NO: 55, 64, 65, or 66.

In some embodiments, the pharmaceutical composition according to any one of the above is provided, wherein the anti-CTGF antibody comprises:
(g) a heavy chain having at least 85%, 90%, 91%, 92%, 93%, 94%, 95%, 96%, 97%, 98%, 99%, or 100% sequence identity to SEQ ID NO: 86, 88, 89, 90, 91, 92, 93, 94, 95, 96, or 97, and a light chain having at least 85%, 90%, 91%, 92%, 93%, 94%, 95%, 96%, 97%, 98%, 99%, or 100% sequence identity to SEQ ID NO: 87, 98, 99, 100, or 101; or
(h) a heavy chain set forth in SEQ ID NO: 86, 88, 89, 90, 91, 92, 93, 94, 95, 96, or 97 and a light chain set forth in SEQ ID NO: 87, 98, 99, 100, or 101.

In some embodiments, the pharmaceutical composition according to any one of the above is provided, wherein the anti-CTGF antibody comprises:
(j) a heavy chain set forth in SEQ ID NO: 46 and a light chain set forth in SEQ ID NO: 49;
(k) a heavy chain set forth in SEQ ID NO: 61 and a light chain set forth in SEQ ID NO: 64; or
(l) a heavy chain set forth in SEQ ID NO: 97 and a light chain set forth in SEQ ID NO: 98.

In some embodiments, the pharmaceutical composition according to any one of the above is provided, wherein the pharmaceutical composition further comprises a buffer. In some embodiments, the buffer is a histidine salt buffer, an acetate buffer, a citrate buffer, a succinate buffer, or a phosphate buffer. In some embodiments, the buffer is a histidine-hydrochloride buffer or a histidine-acetate buffer. In some embodiments, the buffer is a histidine-hydrochloride buffer.

In some embodiments, the pharmaceutical composition according to any one of the above is provided, wherein the pharmaceutical composition has a pH of about 5.0 to about 6.5. In some embodiments, the pharmaceutical composition has a pH of about 5.0 to about 6.0. In some embodiments, the pharmaceutical composition has a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, or about 5.5. In some embodiments, the pharmaceutical composition has a pH of 5.0-6.5. In some embodiments, the pharmaceutical composition has a pH of 5.0-6.0. In some embodiments, the pharmaceutical composition has a pH of 5.5-5.7. In some embodiments, the pharmaceutical composition has a pH of 5.0, 5.1, 5.2, 5.3, 5.4, 5.5, 5.6, 5.7, 5.8, 5.9, 6.0, 6.1, 6.2, 6.3, 6.4, or 6.5, or any range between these point values.

In some embodiments, the pharmaceutical composition according to any one of the above is provided, wherein the anti-CTGF antibody is at a concentration of about 100 mg/mL to about 200 mg/mL. In some embodiments, the anti-CTGF antibody is at a concentration of about 150 mg/mL to about 200 mg/mL. In some embodiments, the anti-CTGF antibody is at a concentration of about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, or about 200 mg/mL. In some embodiments, the anti-CTGF antibody is at a concentration of 100 mg/mL to 200 mg/mL. In some embodiments, the anti-CTGF antibody is at a concentration of 150 mg/mL to 200 mg/mL. In some embodiments, the anti-CTGF antibody is at a concentration of 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, or 200 mg/mL, or any range between these point values.

In some embodiments, the pharmaceutical composition according to any one of the above is provided, wherein the pharmaceutical composition further comprises a surfactant. In some embodiments, the surfactant is a nonionic surfactant. In some embodiments, the surfactant is selected from the group consisting of polysorbate, polysorbate 20, polysorbate 80, poloxamer, Triton, sodium dodecyl sulfonate, sodium lauryl sulfonate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauramido propyl-betaine, cocaramide propyl-betaine, linoleinamide propyl-betaine, myristylamide propyl-betaine, palmitamide propyl-betaine, isostearamide propyl-betaine, myristylamide propyl-dimethylamine, palmitamide propyl-dimethylamine, isostearamide propyl-dimethylamine, sodium methyl cocoyl, sodium methyl oleyl taurate, polyethylene glycol, polypropylene glycol, copolymer of ethylene and propylene glycol, and the like. In some embodiments, the surfactant is a polysorbate or poloxamer. In some embodiments, the surfactant is polysorbate 80, polysorbate 20, or poloxamer 188. In some embodiments, the surfactant is polysorbate 80.

In some embodiments, the pharmaceutical composition according to any one of the above is provided, wherein the surfactant is at a concentration of about 0.05 mg/mL to about 1.0 mg/mL. In some embodiments, the surfactant is at a concentration of about 0.2 mg/mL to about 0.6 mg/mL. In some embodiments, the surfactant is at a concentration of about 0.05 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, or about 1.0 mg/mL. In some embodiments, the surfactant is at a concentration of 0.05 mg/mL to 1.0 mg/mL. In some embodiments, the surfactant is at a concentration of 0.2 mg/mL to 0.6 mg/mL. In some embodiments, the surfactant is at a concentration of 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, or 1.0 mg/mL, or any range between these point values.

In some embodiments, the pharmaceutical composition according to any one of the above is provided, wherein the pharmaceutical composition further comprises a sugar. In some embodiments, the sugar is selected from the group consisting of conventional composition (CH₂O)ₙ and derivatives thereof, including monosaccharides, disaccharides, trisaccharides, polysaccharides, sugar alcohols, reducing sugars, non-reducing sugars, and the like. The sugar may be selected from the group consisting of glucose, sucrose, trehalose, lactose, fructose, maltose, dextran, glycerin, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, and the like. In some embodiments, the sugar is selected from one or more of sucrose, mannitol, and trehalose. In some embodiments, the sugar is sucrose.

In some embodiments, the pharmaceutical composition according to any one of the above is provided, wherein the sugar is at a concentration of about 20 mg/mL to about 100 mg/mL. In some embodiments, the sugar is at a concentration of about 40 mg/mL to about 80 mg/mL. In some embodiments, the sugar is at a concentration of about 60 mg/mL to about 80 mg/mL. In some embodiments, the sugar is at a concentration of about 20 mg/mL, about 30 mg/mL, about 40 mg/mL, about 50 mg/mL, about 60 mg/mL, about 70 mg/mL, about 80 mg/mL, about 90 mg/mL, or about 100 mg/mL. In some embodiments, the sugar is at a concentration of 20 mg/mL to 100 mg/mL. In some embodiments, the sugar is at a concentration of 40 mg/mL to 80 mg/mL. In some embodiments, the sugar is at a concentration of 60 mg/mL to 80 mg/mL. In some embodiments, the sugar is at a concentration of 20 mg/mL, 30 mg/mL, 40 mg/mL, 50 mg/mL, 60 mg/mL, 70 mg/mL, 80 mg/mL, 90 mg/mL, or 100 mg/mL, or any range between these point values.

In some embodiments, the pharmaceutical composition according to any one of the above is provided, wherein the buffer is at a concentration of about 5 mM to about 100 mM. In some embodiments, the buffer is at a concentration of about 30 mM to about 70 mM. In some embodiments, the buffer is at a concentration of about 5 mM, about 10 mM, about 20 mM, about 30 mM, about 40 mM, about 50 mM, about 60 mM, about 70 mM, about 80 mM, about 90 mM, or about 100 mM. In some embodiments, the buffer is at a concentration of 5 mM to 100 mM. In some embodiments, the buffer is at a concentration of 30 mM to 70 mM. In some embodiments, the buffer is at a concentration of 5 mM, 10 mM, 20 mM, 30 mM, 40 mM, 50 mM, 60 mM, 70 mM, 80 mM, 90 mM, or 100 mM, or any range between these point values.

In some embodiments, the pharmaceutical composition according to any one of the above is provided, wherein the pharmaceutical composition further comprises an additional stabilizer. In some embodiments, the additional stabilizer is optionally a salt, a sugar alcohol, a surfactant, a polyether, an amino acid, a chelating agent, or the like. In some embodiments, the additional stabilizer is selected from the group consisting of PEG (polyethylene glycol), arginine, and EDTA. In some embodiments, the PEG is PEG 3350 or PEG 4000. In some embodiments, the PEG is at a concentration of 10 mg/mL to 50 mg/mL. In some embodiments, the PEG is at a concentration of 10 mg/mL, 20 mg/mL, 30 mg/mL, 40 mg/mL, or 50 mg/mL. In some embodiments, the arginine is at a concentration of 10 mM to 100 mM. In some embodiments, the arginine is at a concentration of 10 mM, 20 mM, 30 mM, 40 mM, 50 mM, 60 mM, 70 mM, 80 mM, 90 mM, or 100 mM. In some embodiments, the EDTA is at a concentration of 0.5 mM to 10 mM. In some embodiments, the EDTA is at a concentration of 0.5 mM, 1 mM, 2 mM, 3 mM, 4 mM, 5 mM, 6 mM, 7 mM, 8 mM, 9 mM, or 10 mM, or any range between these point values.

In some embodiments, the pharmaceutical composition according to any one of the above comprises the following components:
(a) the anti-CTGF antibody at about 100 mg/mL to about 200 mg/mL, (b) about 0.05 mg/mL to about 1 mg/mL surfactant, (c) about 20 mg/mL to about 100 mg/mL sugar, and (d) about 5 mM to about 100 mM histidine salt buffer; the pharmaceutical composition having a pH of about 5.0 to about 6.5.

In some embodiments, the pharmaceutical composition according to any one of the above comprises the following components:
(a) the anti-CTGF antibody at about 150 mg/mL to about 200 mg/mL, (b) about 0.2 mg/mL to about 0.6 mg/mL polysorbate, (c) about 40 mg/mL to about 80 mg/mL sugar, and (d) about 30 mM to about 70 mM histidine salt buffer; the pharmaceutical composition having a pH of about 5.0 to about 6.0.

In some embodiments, the pharmaceutical composition according to any one of the above comprises the following components:
(a) the anti-CTGF antibody at about 150 mg/mL, (b) about 0.4 mg/mL polysorbate 80, (c) about 70 mg/mL sucrose, and (d) about 50 mM histidine-hydrochloride buffer; the pharmaceutical composition having a pH of about 5.5 to about 5.7.

In some embodiments, the pharmaceutical composition according to any one of the above comprises the following components:
(a) the anti-CTGF antibody at about 150 mg/mL, (b) about 0.4 mg/mL polysorbate 80, (c) about 70 mg/mL sucrose, and (d) about 50 mM histidine-hydrochloride buffer; the pharmaceutical composition having a pH of about 5.5.

In some embodiments, the pharmaceutical composition according to any one of the above comprises the following components:
(a) the anti-CTGF antibody at 150 mg/mL, (b) 0.4 mg/mL polysorbate 80, (c) 70 mg/mL sucrose, and (d) 50 mM histidine-hydrochloride buffer; the pharmaceutical composition having a pH of 5.5 to 5.7.

In some embodiments, the pharmaceutical composition according to any one of the above comprises the following components:
(a) the anti-CTGF antibody at 150 mg/mL, (b) 0.4 mg/mL polysorbate 80, (c) 70 mg/mL sucrose, and (d) 50 mM histidine-hydrochloride buffer; the pharmaceutical composition having a pH of 5.5.

In some embodiments, the pharmaceutical composition according to any one of the above comprises the following components:
(a) the anti-CTGF antibody at 200 mg/mL, (b) 0.4 mg/mL polysorbate 80, (c) 70 mg/mL sucrose, and (d) 50 mM acetate buffer; the pharmaceutical composition having a pH of 5.0-5.5.

In some embodiments, the pharmaceutical composition according to any one of the above comprises the following components:
(a) the anti-CTGF antibody at 200 mg/mL, (b) 0.4 mg/mL polysorbate 80, (c) 70 mg/mL sucrose, and (d) 50 mM histidine-acetate buffer; the pharmaceutical composition having a pH of 5.5.

In some embodiments, the pharmaceutical composition according to any one of the above comprises the following components:
(a) the anti-CTGF antibody at 200 mg/mL, (b) 0.4 mg/mL polysorbate 80, (c) 70 mg/mL sucrose, and (d) 50 mM succinate buffer; the pharmaceutical composition having a pH of 5.5.

In some embodiments, the pharmaceutical composition according to any one of the above comprises the following components:
(a) the anti-CTGF antibody at 200 mg/mL, (b) 0.4 mg/mL polysorbate 80, (c) 70 mg/mL sucrose, and (d) 50 mM citrate buffer; the pharmaceutical composition having a pH of 5.5.

In some embodiments, the pharmaceutical composition according to any one of the above comprises the following components:
(a) the anti-CTGF antibody at 200 mg/mL, (b) 0.4 mg/mL polysorbate 80, (c) 70 mg/mL sucrose, and (d) 50 mM histidine-hydrochloride buffer; the pharmaceutical composition having a pH of 5.5-6.5. In some embodiments, the pharmaceutical composition has a pH of 5.5, 6.0, or 6.5.

In some embodiments, the pharmaceutical composition according to any one of the above comprises the following components:
(a) the anti-CTGF antibody at 200 mg/mL, (b) 0.4 mg/mL polysorbate 80, (c) 70 mg/mL sucrose, and (d) 50 mM phosphate buffer; the pharmaceutical composition having a pH of 6.0-6.5.

In some embodiments, the pharmaceutical composition according to any one of the above comprises the following components:
(a) the anti-CTGF antibody at 150 mg/mL, (b) 0.4 mg/mL polysorbate 80, (c) 70 mg/mL sucrose, and (d) 50 mM histidine-hydrochloride buffer; the pharmaceutical composition having a pH of 6.0.

In some embodiments, the pharmaceutical composition according to any one of the above comprises the following components:
(a) the anti-CTGF antibody at 150 mg/mL, (b) 0.6 mg/mL polysorbate 20, (c) 70 mg/mL sucrose, and (d) 50 mM histidine-hydrochloride buffer; the pharmaceutical composition having a pH of 5.5.

In some embodiments, the pharmaceutical composition according to any one of the above comprises the following components:
(a) the anti-CTGF antibody at 150 mg/mL, (b) 0.2 mg/mL polysorbate 80, (c) 70 mg/mL sucrose, and (d) 50 mM histidine-hydrochloride buffer; the pharmaceutical composition having a pH of 5.5.

In some embodiments, the pharmaceutical composition according to any one of the above comprises the following components:
(a) the anti-CTGF antibody at 150 mg/mL, (b) 0.6 mg/mL polysorbate 80, (c) 70 mg/mL sucrose, and (d) 50 mM histidine-hydrochloride buffer; the pharmaceutical composition having a pH of 5.5.

In some embodiments, the pharmaceutical composition according to any one of the above comprises the following components:
(a) the anti-CTGF antibody at 150 mg/mL, (b) 0.4 mg/mL poloxamer 188, (c) 70 mg/mL sucrose, and (d) 50 mM histidine-hydrochloride buffer; the pharmaceutical composition having a pH of 5.5.

In some embodiments, the pharmaceutical composition according to any one of the above comprises the following components:
(a) the anti-CTGF antibody at 150 mg/mL, (b) 0.4 mg/mL poloxamer 188, (c) 70 mg/mL sucrose, and (d) 50 mM histidine-hydrochloride buffer; the pharmaceutical composition having a pH of 5.5.

In some embodiments, the pharmaceutical composition according to any one of the above comprises the following components:
(a) the anti-CTGF antibody at 150 mg/mL, (b) 0.4 mg/mL polysorbate 80, (c) 70 mg/mL sucrose, (d) 50 mM histidine-hydrochloride buffer, and (e) 2% PEG 3350; the pharmaceutical composition having a pH of 5.5.

In some embodiments, the pharmaceutical composition according to any one of the above comprises the following components:
(a) the anti-CTGF antibody at 150 mg/mL, (b) 0.4 mg/mL polysorbate 80, (c) 70 mg/mL sucrose, (d) 50 mM histidine-hydrochloride buffer, and (e) 50 mM arginine; the pharmaceutical composition having a pH of 5.5.

In some embodiments, the pharmaceutical composition according to any one of the above comprises the following components:
(a) the anti-CTGF antibody at 150 mg/mL, (b) 0.4 mg/mL polysorbate 80, (c) 70 mg/mL sucrose, (d) 50 mM histidine-hydrochloride buffer, and (e) 1% mannitol; the pharmaceutical composition having a pH of 5.5.

In some embodiments, the pharmaceutical composition according to any one of the above comprises the following components:
(a) the anti-CTGF antibody at 150 mg/mL, (b) 0.4 mg/mL polysorbate 80, (c) 70 mg/mL sucrose, (d) 50 mM histidine-hydrochloride buffer, and (e) 0.5-10 mM EDTA; the pharmaceutical composition having a pH of 5.5. In some embodiments, the EDTA is at a concentration of 0.5 mM, 5 mM, or 10 mM.

In some embodiments, the pharmaceutical composition according to any one of the above comprises the following components:
(a) the anti-CTGF antibody at 150 mg/mL, (b) 0.4 mg/mL polysorbate 80, (c) 70 mg/mL trehalose, and (d) 50 mM histidine-hydrochloride buffer; the pharmaceutical composition having a pH of 5.5.

In some embodiments, the pharmaceutical composition according to any one of the above is provided, wherein the pharmaceutical composition is a liquid formulation. In some embodiments, the liquid formulation contains water as solvent.

The present disclosure further provides a lyophilized formulation, wherein the lyophilized formulation can form, upon reconstitution, the pharmaceutical composition according to any one of the above.

The present disclosure further provides a method for preparing a lyophilized formulation, which comprises a step of lyophilizing the pharmaceutical composition according to any one of the above.

The present disclosure further provides a lyophilized formulation, which is obtained by lyophilizing the pharmaceutical composition according to any one of the above. In some embodiments, the lyophilization according to any one of the above comprises steps of pre-freezing, primary drying, and secondary drying in sequence. In some embodiments, the lyophilized formulation is stable at 40° C. for at least 7 days, at least 14 days, or at least 28 days.

The present disclosure further provides a reconstituted solution, wherein the reconstituted solution is prepared by reconstituting the lyophilized formulation according to any one of the above; optionally, the reconstituted solution is obtained by reconstituting the lyophilized formulation according to any one of the above in a physiologically acceptable solvent, including but not limited to water for injection, normal saline, and buffers.

In some embodiments, the reconstituted solution described above comprises:
(a) the anti-CTGF antibody at about 150 mg/mL to about 200 mg/mL, (b) about 0.2 mg/mL to about 0.6 mg/mL polysorbate, (c) about 40 mg/mL to about 80 mg/mL sugar, and (d) about 30 mM to about 70 mM histidine salt buffer; the pharmaceutical composition having a pH of about 5.0 to about 6.0.

In some embodiments, the reconstituted solution described above comprises:
(a) the anti-CTGF antibody at about 150 mg/mL, (b) about 0.4 mg/mL polysorbate 80, (c) about 70 mg/mL sucrose, and (d) about 50 mM histidine-hydrochloride buffer; the pharmaceutical composition having a pH of about 5.5 to about 5.7.

In some embodiments, the reconstituted solution described above comprises:
(a) the anti-CTGF antibody at about 150 mg/mL, (b) about 0.4 mg/mL polysorbate 80, (c) about 70 mg/mL sucrose, and (d) about 50 mM histidine-hydrochloride buffer; the pharmaceutical composition having a pH of about 5.5.

In some embodiments, the pharmaceutical composition or reconstituted solution according to any one of the above is a formulation for intravenous, subcutaneous, intraperitoneal, or intramuscular injection; in some embodiments, the pharmaceutical composition or reconstituted solution according to any one of the above is a formulation for intravenous injection. In some embodiments, the pharmaceutical composition or reconstituted solution according to any one of the above is suitable for intravenous, subcutaneous, intraperitoneal, or intramuscular injection. In some embodiments, the pharmaceutical composition or reconstituted solution or lyophilized formulation according to any one of the above is for use in the preparation of a medicament for intravenous, subcutaneous, intraperitoneal, or intramuscular injection.

The present disclosure further provides an article of manufacture, which comprises a container containing the pharmaceutical composition according to any one of the above, the lyophilized formulation according to any one of the above, or the reconstituted solution according to any one of the above.

In some embodiments, the present disclosure further provides use of the pharmaceutical composition according to any one of the above, the lyophilized formulation according to any one of the above, the reconstituted solution according to any one of the above, or the article of manufacture according to any one of the above in the preparation of a medicament for treating a CTGF-associated disease.

The present disclosure further provides a method for treating or preventing a CTGF-associated disease, which comprises administering to a patient an effective amount of the pharmaceutical composition according to any one of the above, the lyophilized formulation according to any one of the above, the reconstituted solution according to any one of the above, or the article of manufacture according to any one of the above.

In some embodiments, the present disclosure further provides the pharmaceutical composition according to any one of the above, the lyophilized formulation according to any one of the above, the reconstituted solution according to any one of the above, or the article of manufacture according to any one of the above for use in the treatment of a CTGF-associated disease.

In some embodiments, the CTGF-associated disease is a fibrotic disease, hypertension, diabetes, myocardial infarction, arthritis, a CTGF-associated disease of cellular proliferation, atherosclerosis, glaucoma, or cancer. In some embodiments, the fibrotic disease is selected from the group consisting of: idiopathic pulmonary fibrosis, diabetic nephropathy, diabetic retinopathy, osteoarthritis, scleroderma, chronic heart failure, liver cirrhosis, and renal fibrosis. In some embodiments, the cancer is selected from the group consisting of: acute lymphoblastic leukemia, dermatofibroma, breast cancer, angiolipoma, angioleiomyoma, desmoplastic cancer, prostate cancer, ovarian cancer, colorectal cancer, pancreatic cancer, gastrointestinal cancer, and liver cancer.

### BRIEF DESCRIPTION OF THE DRAWINGS

FIG. 1: the affinity of a humanized antibody derived from mab164 for human CTGF, as measured by ELISA.

FIG. 2: the experimental results of the inhibition of SU86.86 xenograft tumors in mice by a humanized antibody derived from mab147 or mab164.

### DETAILED DESCRIPTION

### Terms

In order to facilitate the understanding of the present disclosure, some technical and scientific terms are specifically defined below. Unless otherwise specifically defined herein, all other technical and scientific terms used herein have the meanings generally understood by those of ordinary skill in the art to which the present disclosure belongs.

The three-letter and single-letter codes for amino acids used herein are as described in J. biol. chem, 243, p3558 (1968).

Connective tissue growth factor (CTGF) is a 36 kD, cysteine-rich, heparin-binding, secreted glycoprotein originally isolated from human umbilical vein endothelial cells. CTGF belongs to the CCN (CTGF, Cyr61, Nov) family of proteins (secreted glycoproteins), which includes the serum-induced immediate early gene product Cyr61, the putative oncogene Nov, the ECM-associated protein FISP-12, the src-induced gene CEF-10, the Wnt-induced secreted protein WISP-3, and the anti-proliferative protein HICP/rCOP. CCN proteins are characterized by 38 conserved cysteine residues that make up more than 10% of the total amino acid content and form a modular structure with N-terminal and C-terminal domains. The modular structure of CTGF includes conserved motifs for insulin-like growth factor binding protein (IGF-BP) and von Willebrand factor (VWC) in the N-terminal domain, and thrombospondin (TSP1) and a cysteine-knot motif in the C-terminal domain. The CTGF of the present disclosure includes wild-type proteins of any origin or variants that retain the function thereof.

The term “antibody” of the present disclosure is used in its broadest sense and encompasses a variety of antibody structures, including but not limited to monoclonal antibodies, polyclonal antibodies, multispecific antibodies (e.g., bispecific antibodies), full-length antibodies or antigen-binding fragments thereof (also known as “antigen-binding moieties”), murine antibodies, chimeric antibodies or humanized antibodies, or affinity-matured antibodies, so long as they exhibit the desired antigen-binding activity. Native antibodies refer to naturally-occurring immunoglobulin molecules. For example, native IgG antibodies are heterotetrameric glycoproteins of about 150,000 Daltons composed of two identical light chains and two identical heavy chains that are disulfide-bonded. From N- to C-terminus, each heavy chain has a variable region (VH), also known as variable heavy domain or heavy chain variable domain, followed by three constant domains (CH1, CH2, and CH3). Similarly, from N- to C-terminus, each light chain has a variable region (VL), also known as variable light domain or light chain variable domain, followed by a constant light (CL) domain. “Full-length antibody”, “intact antibody”, and “whole antibody” are used herein interchangeably to refer to an antibody having a structure substantially similar to a native antibody structure or having heavy chains that comprise an Fc region as defined herein. In some embodiments, the full-length antibodies of the present disclosure include full-length antibodies formed by linking a light chain variable region and a heavy chain variable region from the light and heavy chain variable region combinations in Table 1, Table 2, and Table 3 to a light chain constant region and a heavy chain constant region, respectively. Those skilled in the art can select different antibody-derived light chain constant regions and heavy chain constant regions according to actual needs, for example, human antibody-derived light chain constant regions and heavy chain constant regions.

“Variable region” or “variable domain” refers to a domain in the heavy or light chain of an antibody that is involved in the binding of the antibody to an antigen. Each VH and VL comprises four conserved framework regions (FRs) and three complementarity-determining regions (CDRs). The term “complementarity-determining region” or “CDR” refers to the regions within a variable domain that primarily contribute to antigen binding; “framework” or “FR” refers to variable domain residues other than CDR residues. A VH comprises 3 CDR regions: HCDR1, HCDR2, and HCDR3; a VL comprises 3 CDR regions: LCDR1, LCDR2, and LCDR3. Each VH and VL consists of three CDRs and four FRs arranged from amino-terminus to carboxy-terminus in the following order: FR1, CDR1, FR2, CDR2, FR3, CDR3, and FR4. A single VH or VL may be sufficient to confer antigen-binding specificity.

The boundaries of the amino acid sequences of CDRs can be determined using a variety of well-known schemes, such as the “Kabat” numbering scheme (see Kabat et al., Sequences of Proteins of Immunological Interest, 5th edition, Public Health Service, National Institutes of Health, Bethesda, M D, 1991), the “Chothia” numbering scheme, the “AbM” numbering scheme, the “contact” numbering scheme (see Martin, ACR. Protein Sequence and Structure Analysis of Antibody Variable Domains[J]. 2001), and the ImMunoGenTics (IMGT) numbering scheme (see Lefranc M. P., Dev. Comp. Immunol., 27, 55-77(2003)). The relationships between the numbering schemes including, for example, the Kabat numbering scheme and the IMGT numbering scheme, are well known to those skilled in the art and are shown in Table 4 below.

TABLE 4
The relationships between the numbering schemes for CDRs

| | IMGT | Kabat | AbM | Chothia | Contact |
|---|---|---|---|---|---|
| HCDR1 | 27-38 | 31-35 | 26-35 | 26-32 | 30-35 |
| HCDR2 | 50-65 | 50-65 | 50-58 | 52-56 | 47-58 |
| HCDR3 | 105-117 | 95-102 | 95-102 | 95-102 | 93-101 |
| LCDR1 | 27-38 | 24-34 | 24-34 | 24-34 | 30-36 |
| LCDR2 | 50-65 | 50-56 | 50-56 | 50-56 | 46-55 |
| LCDR3 | 105-117 | 89-97 | 89-97 | 89-97 | 89-96 |

The “class” of an antibody refers to the type of the constant region its heavy chains contain. According to the amino acid sequences of the constant regions, the light chains of antibodies fall into two categories: kappa (κ) and lambda (λ). According to differences in the amino acid composition and the order of arrangement of the heavy chain constant regions of antibodies, antibodies can be divided into five classes, otherwise called antibody isotypes, namely IgM, IgD, IgG, IgA, and IgE, the corresponding heavy chains of which are p chain, 6 chain, y chain, a chain, and F chain, respectively. Ig of the same class can be divided into different subclasses according to differences in the amino acid composition of the hinge regions and the number and positions of disulfide bonds of the heavy chains; for example, IgG can be divided into IgG₁, IgG₂, IgG₃, and IgG₄. Each of the five classes of Ig may have a κ chain or λ chain. The “conventional variant” of the human antibody heavy chain constant region and the human antibody light chain constant region described herein refers to a variant of the heavy chain constant region or light chain constant region derived from humans that has been disclosed in the prior art and does not change the structure and function of the antibody variable region. Exemplary variants include IgG₁, IgG₂, IgG₃, and IgG₄ heavy chain constant region variants with site-directed modifications and amino acid substitutions in the heavy chain constant region. In some embodiments, the substitutions are YTE mutations (M252Y/S254T/T256E), the L234A mutation and/or the L235A mutation, the S228P mutation, and/or mutations that give rise to knob-into-hole structures. These mutations have been shown to confer new properties on antibodies without altering the function of the variable regions of the antibodies. In some embodiments, the antibody is of the IgG₁ isotype, with the P329, P234, and P235 mutations in the hinge region to reduce effector function. In some embodiments, the antibody is of the IgG₂ isotype. In some embodiments, the antibody is of the IgG₄ isotype, with the S228P mutation in the hinge region to improve the stability of the IgG₄ antibody.

“Antibody fragment” refers to a molecule different from an intact antibody; it comprises a portion of an intact antibody, and the portion binds to an antigen to which the intact antibody binds. Examples of antibody fragments include, but are not limited to, Fv, Fab, Fab′, Fab′-SH, F(ab′)₂, single-domain antibodies, diabodies, linear antibodies, single-chain antibody molecules (e.g., scFv), and multispecific antibodies formed from antibody fragments.

“Fc region” or “fragment crystallizable region” is used to define the C-terminal region of the heavy chain of an antibody, including native sequence Fc regions and variant Fc regions. In some embodiments, the Fc region of the human IgG heavy chain is defined as extending from the amino acid residue at position Cys226 or from Pro230 to its carboxy-terminus. The boundaries of the Fc region of the heavy chain of an antibody may also be varied, for example, by deleting the C-terminal lysine of the Fc region (residue 447 according to the EU numbering scheme) or deleting the C-terminal glycine and lysine of the Fc region (residues 446 and 447 according to the EU numbering scheme). Accordingly, in some embodiments, a composition of intact antibodies may comprise antibody populations with all K447 residues and/or G446+K447 residues removed. In some embodiments, a composition of intact antibodies may comprise antibody populations without K447 residues and/or G446+K447 residues removed. In some embodiments, a composition of intact antibodies comprises antibody populations having a mixture of antibodies with and without K447 residues and/or G446+K447 residues. Suitable native sequence Fc regions for the antibodies described herein include human IgG1, IgG2 (IgG2A, IgG2B), IgG3, and IgG4. Unless otherwise specified herein, the numbering of amino acid residues in the Fc region or constant region conforms the EU numbering scheme, also known as the EU index, as described in Kabat et al., Sequences of Proteins of Immunological Interest, 5th edition, Public Health Service, National Institutes of Health, Bethesda, M D, 1991.

“Murine antibody” in the present disclosure is a monoclonal antibody against human CTGF which is derived from mice and is prepared according to the knowledge and skill in the art. During the preparation, a test subject is injected with a CTGF antigen, and then hybridoma of antibodies expressing the desired sequence or functional properties is isolated. In one preferred embodiment of the present disclosure, the murine anti-CTGF antibody or the antigen-binding fragment thereof may further comprise the light chain constant region of a murine κ or λ chain or a variant thereof, or further comprise the heavy chain constant region of murine IgG₁, IgG₂, or IgG₃, or a variant thereof.

The term “chimeric antibody” refers to an antibody obtained by fusing a variable region of a murine antibody and a constant region of a human antibody, which can reduce an immune response induced by the murine antibody. The chimeric antibody is established by firstly establishing hybridoma secreting murine specific monoclonal antibody, then cloning a variable region gene from the mouse hybridoma cells, cloning a constant region gene of human antibody as required, connecting the mouse variable region gene and the human constant region gene into a chimeric gene, inserting the chimeric gene into an expression vector, and finally expressing chimeric antibody molecules in a eukaryotic system or prokaryotic system. In a preferred embodiment of the present disclosure, the antibody light chain of the chimeric antibody further comprises the light chain constant region of a human κ or λ chain or a variant thereof. The antibody heavy chain of the CTGF chimeric antibody further comprises the heavy chain constant region of human IgG1, IgG2, IgG3, or IgG4, or a variant thereof, preferably the heavy chain constant region of human IgG1, IgG2, or IgG4, or an IgG1, IgG2 or IgG4 variant using an amino acid mutation (e.g., an L234A and/or L235A mutation, and/or an S228P mutation).

“Humanized antibody”, also known as a CDR-grafted antibody, refers to an antibody produced by grafting murine CDR sequences into a human antibody variable region framework, i.e., a different type of antibody framework sequence. Such an antibody can overcome the heterogeneous reaction induced by the chimeric antibody because of carrying a large amount of mouse protein component. Such framework sequences can be obtained from public DNA databases or published references that include germline antibody gene sequences. For example, germline DNA sequences of genes of the human heavy and light chain variable regions can be found in the “VBase” human species sequence database, as well as in Kabat, E. A. et al., 1991 Sequences of Proteins of Immunological Interest, 5th edition. To avoid the decrease in activity caused by the decrease in immunogenicity, the framework sequence in human antibody variable region can be subjected to minimum reverse mutation or back mutation to maintain or enhance activity. The humanized antibody of the present disclosure also includes humanized antibodies that were further subjected to CDR affinity maturation mutation by yeast display.

“Affinity” refers to the overall strength of a non-covalent interaction between a single binding site of a molecule (e.g., an antibody) and its binding partner (e.g., an antigen). Unless otherwise indicated, as used herein, “binding affinity” refers to an internal binding affinity that reflects a 1:1 interaction between members of a binding pair (e.g., an antibody and an antigen). The affinity of a molecule X for its ligand Y can be generally expressed by the dissociation constant (KD). Affinity can be determined by conventional methods known in the art, including those described herein. Specific illustrative and exemplary examples for measuring binding affinity are described below.

“kassoc” or “ka” refers to the association rate of a particular antibody-antigen interaction, while the term “kdis” or “kd” as used herein is intended to refer to the dissociation rate of a particular antibody-antigen interaction. As used herein, the term “KD” refers to the dissociation constant, which is obtained from the ratio of kd to ka (i.e., kd/ka) and expressed as a molar concentration (M). The KD value of an antibody can be determined using methods well known in the art. Methods for determining antibody KD include measuring surface plasmon resonance using a biosensing system, such as a system, or measuring affinity in solution by solution equilibrium titration (SET) assay.

An “affinity-matured” antibody is one with one or more alterations in one or more CDRs thereof which result in an improvement in the affinity of the antibody for antigens, compared to the parent antibody which does not contain these alterations. Preferably, in some embodiments, an affinity-matured antibody has nanomolar or even picomolar affinity for the target antigen. Affinity-matured antibodies can be produced using procedures known in the art.

“Amino acid mutation” encompasses amino acid substitutions, deletions, insertions, and modifications. Any combination of substitutions, deletions, insertions, and modifications can be made to arrive at the final construct, provided that the final construct possesses the desired properties. Amino acid sequence deletions and insertions include amino-terminal and/or carboxyl-terminal deletions and amino acid insertions. Particular amino acid mutations may be amino acid substitutions. In one embodiment, amino acid mutations are non-conservative amino acid substitutions—that is, one amino acid is replaced with another amino acid having different structural and/or chemical properties. Amino acid substitutions include replacement with non-naturally occurring amino acids or with derivatives of the 20 native amino acids (e.g., 4-hydroxyproline, 3-methylhistidine, ornithine, homoserine, and 5-hydroxylysine). Amino acid mutations can be generated using genetic or chemical methods well known in the art. Genetic methods may include site-directed mutagenesis, PCR, gene synthesis, and the like. It is contemplated that methods for altering amino acid side chain groups other than genetic engineering, such as chemical modification, may also be used. Various names may be used herein to indicate the same amino acid mutation.

“About” described in the disclosure means that it is within an acceptable error range for a particular value as determined by one of ordinary skill in the art, which will depend in part on how the value is measured or determined, i.e., the limitations of the measurement system. In the context of a particular assay, result or embodiment, “about” means a given value ±a range within 5%, unless otherwise explicitly stated in the example or elsewhere in the specification.

“Buffer” refers to a buffer that resists changes in pH by the action of its acid-base conjugate components. Examples of buffers that control the pH in an appropriate range include acetate, succinate, gluconate, histidine salt, oxalate, lactate, phosphate, citrate, tartrate, fumarate, glycylglycine, and other organic acid buffers.

“Histidine salt buffer” is a buffer comprising histidine ions. Examples of histidine salt buffers include histidine-hydrochloride buffer, histidine-acetate buffer, histidine-phosphate buffer, histidine-sulfate buffer, and the like, and the histidine-hydrochloride buffer or histidine-acetate buffer is preferred. The histidine-acetate buffer is prepared with histidine and acetic acid, and the histidine hydrochloride buffer is prepared with histidine and histidine hydrochloride, or histidine and hydrochloric acid.

“Citrate buffer” is a buffer comprising citrate ions. Examples of citrate buffers include citric acid-sodium citrate, citric acid-potassium citrate, citric acid-calcium citrate, citric acid-magnesium citrate, and the like. The preferred citrate buffer is citric acid-sodium citrate.

“Succinate buffer” is a buffer comprising succinate ions. Examples of succinate buffers include succinic acid-sodium succinate, succinic acid-potassium succinate, succinic acid-calcium succinate, and the like. The preferred succinate buffer is succinic acid-sodium succinate. Illustratively, the succinic acid-sodium succinate may be prepared with succinic acid and sodium hydroxide, or succinic acid and sodium succinate.

“Phosphate buffer” is a buffer comprising phosphate ions. Examples of phosphate buffers include disodium hydrogen phosphate-sodium dihydrogen phosphate, disodium hydrogen phosphate-potassium dihydrogen phosphate, disodium hydrogen phosphate-citric acid, and the like. The preferred phosphate buffer is disodium hydrogen phosphate-sodium dihydrogen phosphate.

“Acetate buffer” is a buffer comprising acetate ions. Examples of acetate buffers include acetic acid-sodium acetate, acetic acid histidine salt, acetic acid-potassium acetate, acetic acid-calcium acetate, acetic acid-magnesium acetate, and the like. The preferred acetate buffer is acetic acid-sodium acetate.

“Stabilizer” refers to a component that helps maintain the structural integrity of a biopharmaceutical drug, particularly during freezing and/or lyophilization and/or storage (particularly when exposed to stress). This stabilizing effect may arise for a variety of reasons, and typically, such stabilizers may act as osmotic agents which reduce protein denaturation. The additional stabilizer herein refers to a stabilizer other than buffer systems, surfactants, and sugar alcohols, for example, a stabilizer selected from the group consisting of PEG, arginine, and EDTA.

“Pharmaceutical composition” refers to a mixture comprising one or more of the antibodies described herein and other chemical components, for example, physiological/pharmaceutically acceptable carriers and excipients. The purpose of the pharmaceutical composition is to maintain the stability of the active ingredient and promote the administration to an organism, which facilitates the absorption of the active ingredient, thereby exerting biological activity.

As used herein, “pharmaceutical composition” and “formulation” are not mutually exclusive.

Unless otherwise stated, the solvent in the pharmaceutical composition described herein in solution form is water.

“Lyophilized formulation” refers to a formulation or a pharmaceutical composition obtained by freeze-drying a pharmaceutical composition in liquid or solution form or a liquid or solution formulation in vacuum.

While the present disclosure provides content ranges or values, those of ordinary skill in the art will appreciate that the content ranges or values encompass acceptable error ranges for the particular values determined.

The pharmaceutical composition described herein can achieve an effect of being stable: a pharmaceutical composition in which the antibody substantially retains its physical and/or chemical stability and/or biological activity after storage; preferably, the pharmaceutical composition substantially retains its physical and chemical stability as well as its biological activity after storage. The storage period is generally selected based on a predetermined shelf life of the pharmaceutical composition. There are a variety of analytical techniques currently available for determining protein stability, and the stability after storage for a selected period of time at a selected temperature can be determined.

A stable formulation is one in which no significant change is observed under the following conditions: stored at refrigeration temperature (2-8° C.) for at least 3 months, preferably 6 months, more preferably 1 year, and even more preferably up to 2 years. In addition, stable liquid formulations include liquid formulations that exhibit desirable features after storage at temperatures including 25° C. for periods including 1 month, 3 months, and 6 months. Typical examples for stability are as follows: typically, no more than about 10%, preferably no more than about 5%, of antibody monomers aggregate or are degraded as measured by SEC-HPLC. The formulation is a pale yellow, nearly colorless and clear liquid, or a colorless and clear liquid, or is clear to slightly opalescent, by visual analysis. The concentration, pH, and osmolality of the formulation have no more than ±10% change. Typically, no more than about 10%, preferably no more than about 5%, of decrease is observed. Typically, no more than about 10%, preferably no more than about 5%, of aggregation is formed.

An antibody “retains its physical stability” in a pharmaceutical formulation if it shows no significant increase in aggregation, precipitation and/or denaturation upon visual inspection of color and/or clarity, or as determined by UV light scattering, size exclusion chromatography (SEC), and dynamic light scattering (DLS). Changes in protein conformation can be assessed by fluorescence spectroscopy (which determines the protein tertiary structure) and by FTIR spectroscopy (which determines the protein secondary structure).

An antibody “retains its chemical stability” in a pharmaceutical formulation if it shows no significant chemical change. Chemical stability can be assessed by detecting and quantifying chemically changed forms of the protein. Degradation processes that often change the chemical structure of proteins include hydrolysis or clipping (evaluated by methods such as size exclusion chromatography and CE-SDS), oxidation (evaluated by methods such as peptide mapping in conjunction with mass spectroscopy or MALDI/TOF/MS), deamidation (evaluated by methods such as ion-exchange chromatography, capillary isoelectric focusing, peptide mapping, and isoaspartic acid measurement), and isomerization (evaluated by measuring the isoaspartic acid content, peptide mapping, etc.).

An antibody “retains its biological activity” in a pharmaceutical formulation if the biological activity of the antibody at a given time is within a predetermined range of the biological activity exhibited during the preparation of the pharmaceutical formulation.

“Administrating”, “giving”, and “treating”, when applied to animals, humans, experimental subjects, cells, tissues, organs or biological fluids, refer to contact of an exogenous drug, a therapeutic agent, a diagnostic agent or a composition with the animals, humans, subjects, cells, tissues, organs or biological fluids. “Administering”, “giving”, and “treating” can refer to, for example, therapeutic, pharmacokinetic, diagnostic, research, and experimental methods. The treatment of cells comprises making the reagent in contact with the cells and making the reagent in contact with fluid, where the fluid is in contact with the cells. “Administering”, “giving”, and “treating” also refer to treating, for example, cells by reagents, diagnosis, binding compositions or by another cell in vitro and ex vivo. “Treating”, when applied to humans, veterinary, or research subjects, refers to therapeutic treatment, preventive or prophylactic measures, and research and diagnostic applications.

“Treatment” refers to administering a therapeutic agent, for example, comprising any one of the pharmaceutical compositions of the present disclosure, either internally or externally to a patient with one or more symptoms of a disease on which the therapeutic agent is known to have a therapeutic effect. Generally, the therapeutic agent is administered in an amount effective to alleviate one or more symptoms of the disease in the patient or population being treated to induce regression of such symptoms or inhibit the development of such symptoms to any clinically measurable degree. The amount of therapeutic agent effective to alleviate the symptoms of any particular disease (also known as a “therapeutically effective amount”) may vary depending on a variety of factors, such as the disease state, age, and weight of the patient, and the ability of the drug to produce a desired therapeutic effect in the patient. Whether a symptom of a disease has been alleviated can be evaluated by any clinical testing methods commonly used by doctors or other health care professionals to evaluate the severity or progression of the symptom. Although the embodiments of the present disclosure (for example, treatment methods or articles of manufacture) may not be effective in alleviating the symptoms of each disease of interest, they shall reduce the symptoms of a disease of interest in a statistically significant number of patients, as determined according to any statistical testing methods known in the art, such as Student t-test, chi-square test, Mann and Whitney's U test, Kruskal-Wallis test (H test), Jonckheere-Terpstra test, and Wilcoxon test.

There is no limitation for a CTGF-associated disease in the present disclosure, as long as it is a disease associated with CTGF. For example, the therapeutic response induced by the molecule of the present disclosure can be achieved by binding human CTGF and then blocking the binding of CTGF to its receptors/ligands, or killing tumor cells over-expressing CTGF. Thus, the molecules of the present disclosure are very useful, when in preparations and formulations suitable for therapeutic applications, for people who have tumors or cancer, optionally including lung fibrosis, kidney fibrosis, tumor formation and growth, glaucoma, diseases of cell proliferation, cataracts, choroidal neovascularization, retinal detachment, proliferative vitreoretinopathy, macular degeneration, diabetic retinopathy, corneal scarring and corneal haze, cysts, reduced vascular calcification, pancreatic ductal adenocarcinoma, pancreatic cancer, melanoma, radiation-induced fibrosis (RIF), idiopathic pulmonary fibrosis, and pulmonary remodeling diseases selected from the group consisting of asthma, chronic bronchitis, chronic obstructive pulmonary disease (COPD), cystic fibrosis, emphysema, and the like, preferably pancreatic cancer, pulmonary fibrosis, and kidney fibrosis.

The details of one or more embodiments of the present disclosure are set forth in the specification above. Although any methods and materials similar or identical to those described herein can be used in the practice or testing of the present disclosure, the preferred methods and materials are described below. Other features, objects, and advantages of the present disclosure will be apparent from the specification and the claims. In the specification and claims, singular forms include plural referents unless otherwise indicated clearly in the context. Unless otherwise defined, all technical and scientific terms used herein have the meanings generally understood by those of ordinary skill in the art to which the present disclosure belongs. All the patents and publications cited in the specification are incorporated by reference. The following examples are set forth in order to more fully illustrate the preferred embodiments of the present disclosure. These examples should not be construed in any way as limiting the scope of the present disclosure, which is defined by the claims.

### EXAMPLES

The present disclosure is further described below with reference to examples, which, however, are not intended to limit the scope of the present disclosure. The experimental methods in the examples of the present disclosure in which specific conditions are not specified are generally performed under conventional conditions, for example, by referring to Antibodies: A Laboratory Manual and Molecular Cloning: A Laboratory Manual by Cold Spring Harbor Laboratory, or under conditions recommended by the manufacturer of the raw material or the goods. Reagents without specific origins indicated were commercially available conventional reagents.

### I. Antibodies

### Example 1-1. Preparation of CTGF Antigen and Antibodies

### I. Antigen Design and Expression

With human CTGF protein (Genbank No. NM_001901.2) as a template, the amino acid sequences of the antigen and the protein for assays were designed; optionally, the CTGF protein or fragments were fused with different tags. The sequences were cloned onto pTT5 vectors or pXC vectors and transiently expressed in 293 cells or stably expressed in LONZA CHO cells to obtain the encoded antigen and protein for assays of the present disclosure. The following CTGF antigens are all human CTGF unless otherwise specified.

Human CTGF full-length protein (for immunization), SEQ ID NO: 1)
QNCSGPCRCPDEPAPRCPAGVSLVLDGCGCCRVCAKQLGELCTERDPCDPHKGL
FCDFGSPANRKIGVCTAKDGAPCIFGGTVYRSGESFQSSCKYQCTCLDGAVGCM
PLCSMDVRLPSPDCPFPRRVKLPGKCCEEWVCDEPKDQTVVGPALAAYRLEDTF
GPDPTMIRANCLVQTTEWSACSKTCGMGISTRVTNDNASCRLEKQSRLCMVRP
CEADLEENIKKGKKCIRTPKISKPIKFELSGCTSMKTYRAKFCGVCTDGRCCTPH
RTTTLPVEFKCPDGEVMKKNMMFIKTCACHYNCPGDNDIFESLYYRKMYGDM
A
CTGF-his (a fusion protein of the human CTGF mature protein and a his
tag), can be used for assays,
SEQ ID NO: 2
MEFGLSWLFLVAILKGVQCQNCSGPCRCPDEPAPRCPAGVSLVLDGCGCCRVCAK
QLGELCTERDPCDPHKGLFCDFGSPANRKIGVCTAKDGAPCIFGGTVYRSGESFQSS
CKYQCTCLDGAVGCMPLCSMDVRLPSPDCPFPRRVKLPGKCCEEWVCDEPKDQTV
VGPALAAYRLEDTFGPDPTMIRANCLVQTTEWSACSKTCGMGISTRVINDNASCRLE
KQSRLCMVRPCEADLEENIKKGKKCIRTPKISKPIKFELSGCTSMKTYRAKFCGVCT
DGRCCTPHRTTTLPVEFKCPDGEVMKKNMMFIKTCACHYNCPGDNDIFESLYYRK
MYGDMAHHHHHH
(Note: the signal peptide is single-underlined, the human CTGF coding
region is italicized, and the his tag is double-underlined.)
mCTGF-mFc (a fusion protein of the mouse CTGF coding region and a
mouse Fc tag), can be used for assays,
SEQ ID NO: 3
MEFGLSWLFLVAILKGVQCQDCSAQCQCAAEAAPHCPAGVSLVLDGCGCCRVCAK
QLGELCTERDPCDPHKGLFCDFGSPANRKIGVCTAKDGAPCVFGGSVYRSGESFQS
SCKYQCTCLDGAVGCVPLCSMDVRLPSPDCPFPRRVKLPGKCCEEWVCDEPKDRT
AVGPALAAYRLEDTFGPDPTMMRANCLVQTTEWSACSKTCGMGISTRVINDNTFCRL
EKQSRLCMVRPCEADLEENIKKGKKCIRTPKIAKPVKFELSGCTSVKTYRAKFCGVC
TDGRCCTPHRTTTLPVEFKCPDGEIMKKNMMFIKTCACHYNCPGDNDIFESLYYRK
MYGDMAEPRGPTIKPCPPCKCPAPNLLGGPSVFIFPPKIKDVLMISLSPIVTCVVV
DVSEDDPDVQISWFVNNVEVHTAQTQTHREDYNSTLRVVSALPIQHQDWMSG
KEFKCKVNNKDLPAPIERTISKPKGSVRAPQVYVLPPPEEEMTKKQVTLTCMVT
DFMPEDIYVEWTNNGKTELNYKNTEPVLDSDGSYFMYSKLRVEKKNWVERNS
YSCSVVHEGLHNHHTTKSFSRTPGK
(Note: the signal peptide is single-underlined, the mouse CTGF coding
region is italicized, and the mFc tag is double-underlined.)
Mouse CTGF protein-His tag,
SEQ ID NO: 4
MEFGLSWLFLVAILKGVQCQDCSAQCQCAAEAAPHCPAGVSLVLDGCGCCRVCAK
QLGELCTERDPCDPHKGLFCDFGSPANRKIGVCTAKDGAPCVFGGSVYRSGESFQS
SCKYQCTCLDGAVGCVPLCSMDVRLPSPDCPFPRRVKLPGKCCEEWVCDEPKDRT
AVGPALAAYRLEDTFGPDPTMMRANCLVQTTEWSACSKTCGMGISTRVINDNTFCRL
EKQSRLCMVRPCEADLEENIKKGKKCIRTPKIAKPVKFELSGCTSVKTYRAKFCGVC
TDGRCCTPHRTTTLPVEFKCPDGEIMKKNMMFIKTCACHYNCPGDNDIFESLYYRK
MYGDMAHHHHHH
(Note: the signal peptide is single-underlined, the mouse CTGF coding
region is italicized, and the his tag is double-underlined.)
Cynomolgus monkey CTGF-Fc (a fusion protein of the cynomolgus monkey
CTGF coding region and Fc), can be used for assays,
SEQ ID NO: 5
MEFGLSWLFLVAILKGVQCQNCSGPCRCPAEQAPRCPAGVSLVLDGCGCCRVCAK
QLGELCTERDPCDPHKGLFCDFGSPANRKIGVCTAKDGAPCIFGGTVYRSGESFQSS
CKYQCTCLDGAVGCMPLCSMDVRLPSPDCPFPRRVKLPGKCCEEWVCDEPKDQTV
VGPALAAYRLEDTFGPDPTMIRANCLVQTTEWSACSKTCGMGISTRVINDNASCRLE
KQSRLCMVRPCEADLEENIKKGKKCIRTPKISKPIKFELSGCTSVKTYRAKFCGVCTD
GRCCTPHRTTTLPVEFKCPDGEVMKKNMMFIKTCACHYNCPGDNDIFESLYYRKM
YGDMAEPKSSDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLTCLVKG
FYPSDIAVEWESNGQPENNYKTTPPVLDSDGSFFLYSKLTVDKSRWQQGNVESC
SVMHEALHNHYTQKSLSLSPGK
(Note: the signal peptide is single-underlined, the cynomolgus monkey
CTGF coding region is italicized. and the Fc tag is double-underlined.)

### II. Purification of CTGF-Associated Recombinant Protein and Purification of Anti-Human CTGF Hybridoma Antibody and Recombinant Antibody

### 1. Separation and Purification of Hybridoma Supernatant by ProteinG Affinity Chromatography

For the purification of mouse hybridoma supernatant, Protein G is preferred for affinity chromatography. The cultured hybridoma was centrifuged to obtain the supernatant, and 10-15% by volume of 1 M Tris-HCl (pH 8.0-8.5) was added based on the volume of the supernatant to adjust the pH of the supernatant to neutrality. The Protein G column was washed with 3-5 column volumes of 6 M guanidine hydrochloride, then with 3-5 column volumes of pure water; the chromatographic column was equilibrated with 3-5 column volumes of, for example, 1×PBS (pH 7.4); the cell supernatant was loaded on the column at a low flow rate for binding, with the flow rate controlled to allow for a retention time of about 1 min or longer; the chromatographic column was washed with 3-5 column volumes of 1×PBS (pH 7.4) until the UV absorbance fell back to baseline; the sample was eluted with a 0.1 M acetic acid/sodium acetate (pH 3.0) buffer, the elution peaks were collected by UV detection, and the eluted product was rapidly adjusted to pH 5-6 with 1 M Tris-HCl (pH 8.0). For the eluted product, solution exchange may be performed by methods well known to those skilled in the art, such as ultrafiltration concentration using an ultrafiltration tube to exchange the solution to a desired buffer system, or exchange with a desired buffer system by size exclusion (e.g., G-25 desalination), or removal of polymer components from the eluted product using a high-resolution molecular exclusion column such as Superdex 200 to improve sample purity.

### 2. Purification of Antibody by Protein a Affinity Chromatography

Firstly, the cell culture supernatant expressing the antibody was centrifuged at high speed, and the supernatant was collected. The Protein A affinity column was washed with 3-5 column volumes of 6 M guanidine hydrochloride, then with 3-5 column volumes of pure water. The chromatographic column was equilibrated with 3-5 column volumes of, for example, 1×PBS (pH 7.4). The cell supernatant was loaded on the column at a low flow rate for binding, with the flow rate controlled to allow for a retention time of about 1 min or longer. After the binding was completed, the chromatographic column was washed with 3-5 column volumes of 1×PBS (pH 7.4) until the UV absorbance fell back to baseline. The sample was eluted with a 0.1 M acetic acid/sodium acetate (pH 3.0-3.5) buffer, the elution peaks were collected by UV detection, and the eluted product was rapidly adjusted to pH 5-6 with 1 M Tris-HCl (pH 8.0). For the eluted product, solution exchange may be performed by methods well known to those skilled in the art, such as ultrafiltration concentration using an ultrafiltration tube to exchange the solution to a desired buffer system, or exchange with a desired buffer system by size exclusion (e.g., G-25 desalination), or removal of polymer components from the eluted product using a high-resolution molecular exclusion column such as Superdex 200 to improve sample purity.

### 3. Nickel Column Purification of Human CTGF-his and Other CTGF-Associated Recombinant Proteins

The cell expression supernatant sample was centrifuged at high speed to remove impurities, and the buffer was exchanged with PBS, followed by the addition of imidazole to make a final concentration of 5 mM. A nickel column was equilibrated with a PBS solution containing 5 mM imidazole and washed with 2-5 column volumes. The supernatant sample after exchange was loaded on the column for binding. Nickel columns from different companies could be selected as the medium. The column was washed with a PBS solution containing 5 mM imidazole until A280 reading dropped to baseline. The chromatographic column was then washed with a mixture of PBS and 10 mM imidazole to remove non-specifically bound impure proteins, and the effluent was collected. The target protein was eluted with a PBS solution containing 300 mM imidazole and the elution peaks were collected.

After being concentrated, the collected eluted product could be further purified by gel chromatography Superdex200 (GE) with PBS as the mobile phase to remove aggregates and impurity protein peaks, and the elution peak of the target product was collected. The obtained protein was identified by electrophoresis, peptide mapping, and LC-MS, and then aliquoted for later use if it was determined to be correct.

### 4. Purification of Human CTGF-Fc, Cynomolgus Monkey CTGF-Fc, and Other CTGF-Associated Recombinant Proteins by Protein a Affinity Chromatography

Firstly, the culture supernatant was centrifuged at high speed, and the supernatant was collected. The Protein A affinity column was washed with 3-5 column volumes of 6 M guanidine hydrochloride, then with 3-5 column volumes of pure water. The chromatographic column was equilibrated with 3-5 column volumes of, for example, 1×PBS (pH 7.4). The cell supernatant was loaded on the column at a low flow rate for binding, with the flow rate controlled to allow for a retention time of about 1 min or longer. After the binding was completed, the chromatographic column was washed with 3-5 column volumes of 1×PBS (pH 7.4) until the UV absorbance fell back to baseline. The sample was eluted with a 0.1 M acetic acid/sodium acetate (pH 3.0-3.5) buffer, and the elution peak was collected by UV detection.

After being concentrated, the collected eluted product could be further purified by gel chromatography Superdex200 (GE) with PBS as the mobile phase to remove aggregates and impurity protein peaks, and the elution peak of the target product was collected. The obtained protein was identified by electrophoresis, peptide mapping, and LC-MS, and then aliquoted for later use if it was determined to be correct.

### Example 1-2. Preparation of Anti-Human CTGF Monoclonal Antibodies

### I. Immunization

Anti-human CTGF monoclonal antibodies were produced by immunizing mice. Laboratory SJL white mice, female, 6-8 weeks of age (Beijing Vital River Laboratory Animal Technology Co., Ltd., animal production license number: SCXK(Beijing)2012-0001). Housing environment: SPF. The purchased mice were housed in a laboratory environment for 1 week, in 12/12 hour light/dark cycles, at a temperature of 20-25° C., with humidity at 40-60%. The acclimatized mice were immunized according to the following scheme. The immune antigens were CTGF (R&D) and mCTGF-mFc.

Immunization scheme: mice were immunized with CTGF protein at 25 g/mouse/time by intraperitoneal injection. On day 0, each mouse was injected intraperitoneally (IP) with 100 μL and was administered a boost immunization every 14 days. Blood was collected on days 21, 35, 49, and 63, and the antibody titer in mouse serum was determined by ELISA. After 7-9 immunizations, mice in which the antibody titer in serum was high and was reaching a plateau were selected for splenocyte fusion. A boost immunization was performed 3 days before the splenocyte fusion, and a solution of CTGF protein antigen in normal saline was intraperitoneally (IP) injected at g/mouse.

### II. Splenocyte Fusion

Spleen lymphocytes and Sp2/0 myeloma cells (ATCC® CRL-8287™) were fused by following a PEG-mediated fusion procedure to obtain hybridoma cells. The resulting hybridoma cells were resuspended in a complete medium (an IMDM medium containing 20% FBS, 1×HAT, and 1×OPI) at a density of 0.5-1×10⁶/mL and seeded in a 96-well plate at 100 μL/well. The plate was incubated at 37° C. with 5% CO₂ for 3-4 days, supplemented with the HAT complete medium at 100 μL/well, and incubated for another 3-4 days to form pinpoint-like clones. The supernatant was removed, and an HT complete medium (an IMDM medium containing 20% FBS, 1×HT, and 1×OPI) was added at 200 μL/well. The plate was incubated at 37° C. with 5% CO₂ for 3 days, followed by an ELISA assay.

### III. Screening of Hybridoma Cells

Hybridoma culture supernatants were tested by a binding ELISA method according to the growth density of hybridoma cells. Then the cell supernatants from positive wells as determined by binding ELISA were subjected to a binding experiment with monkey CTGF/mouse CTGF/human CTGF proteins (using the same method as Example 3). The cells from positive wells as determined by the protein ELISA binding experiment were expanded and cryopreserved in time and subcloned 2 to 3 times until single cell clones were obtained.

A CTGF binding ELISA assay was also performed for each cell subcloning. Hybridoma clones were obtained by the above screening process, and antibodies were further prepared using a serum-free cell culture method. The antibodies were purified, according to the purification example, for use in the test examples.

### IV. Sequencing of Hybridoma Positive Clones

The cloning of sequences from positive hybridomas is as follows. Hybridoma cells in the logarithmic growth phase were harvested, and RNA was extracted with Trizol (Invitrogen, Cat No. 15596-018) by following the procedures in the kit instructions and reverse-transcribed using a PrimeScript™ Reverse Transcriptase kit (Takara, Cat No. 2680A). The cDNA obtained by reverse transcription was amplified by PCR using mouse Ig-Primer Set (Novagen, TB326 Rev.B 0503), and then the products of the amplification were sent to a sequencing company for sequencing. The amino acid sequences of the variable regions of the murine anti-CTGF antibodies mab147, mab164, and mab95 obtained by sequencing are as follows:

The amino acid sequence of the VH of mab 147 is set forth in SEQ ID NO: 6,
EVQLVESGGGLVQPEGSLKLSCAASGFSFNTYAMNWVRQAPGKGLEWVARIRTKSNN
YATYYADSVKDRFTISRDDSESMLYLQMNNLKTEDTAMYYCVETGFAYWDQGTLVTVS
A
The amino acid sequence of the VL of mab 147 is set forth in SEQ ID NO: 7,
QIVLTQSPAIMSASPGEKVTITCSASSSVSYMHWFQQKPGTSPKLWIYSTSNLASGVPAR
FSGSGSGTSYSLTISRMEAEDAATYYCQQRSSYPLTFGAGTKLELK
The amino acid sequence of the VH of mab 164 is set forth in SEQ ID NO: 8,
QVQLKQSGPGLVQPSQSLSITCTVSGFSLTTFGVHWIRQSPGKGLEWLGVIWRRGGT
DYNAAFMSRLSITKDNSRSHVFFKMTSLQTDDSAIYYCARDGGFDYWGQGTTLTVSS
The amino acid sequence of the VL of mab 164 is set forth in SEQ ID NO: 9,
QAVVTQESALTTSPGGTVILTCRSSIGAVTTSNYANWVQEKPDHLFTGLIGGTSNRAPG
VPVRFSGSLIGDKAALTITGAQAEDDAMYFCALWYSTHYVFGGGTKVTVL
The amino acid sequence of the VH of mab95 is set forth in SEQ ID NO: 69,
EVLLQQSGPVLVKPGPSVKISCKASGFTFTNYDIHWVKQSHGKSLEWIGLVYPYNGG
TAYNQKFKDKATLTFDTSSSTAYMELNSLTSEDSAVYYCARWGLIPGTTSYFDVWGTG
TTVTVSS
The amino acid sequence of the VL of mab95 is set forth in SEQ ID NO: 70,
QIVLSQSPPILSASPGEKVTMTCRASSSVSYIHWYQQKPRSSPKPWIYATSNLASGVPAR
FSGSGSGTSYSLTISRVEAEDAATYYCQQWNSNPWTFGGGTRLEIK

In the variable region sequences of the mab147, mab164 and mab95 antibodies, the FR sequences are italicized, the CDR sequences are underlined and italicized, and the sequence order is FR1-CDR1-FR2-CDR2-FR3-CDR3-FR4.

TABLE 5
The amino acid sequences of the CDR regions of the heavy and light
chains of anti-CTGF antibodies*

| Antibody | Heavy chain | | Light chain | |
|---|---|---|---|---|
| mab147 | HCDR1 | TYAMN | LCDR1 | SASSSVSYMH |
| | | SEQ ID NO: 10 | | SEQ ID NO: 13 |
| | HCDR2 | RIRTKSNNYATYYADSVKD | LCDR2 | STSNLAS |
| | | SEQ ID NO: 11 | | SEQ ID NO: 14 |
| | HCDR3 | TGFAY | LCDR3 | QQRSSYPLT |
| | | SEQ ID NO: 12 | | SEQ ID NO: 15 |
| mab164 | HCDR1 | TFGVH | LCDR1 | RSSIGAVTTSNYAN |
| | | SEQ ID NO: 16 | | SEQ ID NO: 19 |
| | HCDR2 | VIWRRGGTDYNAAFMS | LCDR2 | GTSNRAP |
| | | SEQ ID NO: 17 | | SEQ ID NO: 20 |
| | HCDR3 | DGGFDY | LCDR3 | ALWYSTHYV |
| | | SEQ ID NO: 18 | | SEQ ID NO: 21 |
| mab95 | HCDR1 | NYDIH | LCDR1 | RASSSVSYIH |
| | | SEQ ID NO: 71 | | SEQ ID NO: 74 |
| | HCDR2 | LVYPYNGGTAYNQKFKD | LCDR2 | ATSNLAS |
| | | SEQ ID NO: 72 | | SEQ ID NO: 75 |
| | HCDR3 | WGLIPGTTSYFDV | LCDR3 | QQWNSNPWT |
| | | SEQ ID NO: 73 | | SEQ ID NO: 76 |

*The CDRs in the table are determined by the Kabat numbering scheme.

### V. Preparation of Human IgG1 Chimeric Antibodies

The variable region coding gene sequences could be obtained by amplifying and sequencing candidate molecules mab147, mab164, and mab95, a head-tail primer was designed using the sequences obtained by sequencing, a VH/VK gene fragment was constructed for each antibody by PCR using the sequenced gene as a template, and homologously recombined with an expression vector pHr (with a signal peptide and an hIgG1/hkappa/hlambda constant region gene (CH1-Fc/CL) fragment) to construct a recombinant chimeric antibody full-length expression plasmid VH-CH1-Fc-pHr/VL-CL-pHr, and to form three chimeric antibodies: Ch147, Ch164, and Ch95.

### VI. Humanization of Murine Anti-Human CTGF Antibodies

By comparing the IMGT (http://imgt.cines.fr) human antibody heavy and light chain variable region germline gene database with the MOE (molecular operating environment) software, heavy and light chain variable region germline genes with high homology to the murine antibodies were selected as templates, and CDRs of the murine antibodies were grafted into corresponding humanized templates to form variable region sequences in the following order: FR1-CDR1-FR2-CDR2-FR3-CDR3-FR4.

1. Humanization of mab147

The humanization light chain templates for the murine antibody mab147 are IGKV3-11*01 and IGKJ2*01 or IGKV1-39*01 and IGKJ2*01, and the humanization heavy chain templates are IGHV3-72*01 and IGHJ1*01. The CDRs of the murine antibody mab147 were grafted into its humanization templates, and further, some amino acids of the FR of the humanized antibody were modified with back mutations, wherein back mutations for light chain variable regions include one or more of 4L, 36F, 43S, 45K, 47W, 58V, and 71Y, and back mutations for heavy chain variable regions include one or more of 28S, 30N, 49A, 75E, 76S, 93V, 94E, and 104D (the positions of the back mutations in the light and heavy chain variable regions are determined by the Kabat numbering scheme); light and heavy chain variable regions with different sequences were obtained. Thus, a humanized antibody comprising the CDRs of mab147 was obtained, and the variable region sequences thereof are as follows:

The specific sequences of the variable regions of the mAb147 humanized antibody are as follows:

The amino acid sequence of Hu147-VL1 is set forth in SEQ ID NO: 22:
EIVLTQSPATLSLSPGERATLSCSASSSVSYMHWFQQKPGQSPRLLIYSTSNLASGIPARF
SGSGSGTDYTLTISSLEPEDFAVYYCQQRSSYPLTFGQGTKLEIK
The amino acid sequence of Hu147-VL2 is set forth in SEQ ID NO: 23:
EIVLTQSPATLSLSPGERATLSCSASSSVSYMHWFQQKPGQSPKLLIYSTSNLASGIPAR
FSGSGSGTDYTLTISSLEPEDFAVYYCQQRSSYPLTFGQGTKLEIK
The amino acid sequence of Hu147-VL3 is set forth in SEQ ID NO: 24:
EIVLTQSPATLSLSPGERATLSCSASSSVSYMHWFQQKPGQSPKLWIYSTSNLASGVPAR
FSGSGSGTDYTLTISSLEPEDFAVYYCQQRSSYPLTFGQGTKLEIK
The amino acid sequence of Hu147-VL4 is set forth in SEQ ID NO: 25:
DIQMTQSPSSLSASVGDRVTITCSASSSVSYMHWFQQKPGKSPKLLIYSTSNLASGVPS
RFSGSGSGTDYTLTISSLQPEDFATYYCQQRSSYPLTFGQGTKLEIK
The amino acid sequence of Hu147-VL5 is set forth in SEQ ID NO: 26:
DIQLTQSPSSLSASVGDRVTITCSASSSVSYMHWFQQKPGKSPKLWIYSTSNLASGVPS
RFSGSGSGTDYTLTISSLQPEDFATYYCQQRSSYPLTFGQGTKLEIK
The amino acid sequence of Hu147-VH1 is set forth in SEQ ID NO: 27:
EVQLVESGGGLVQPGGSLRLSCAASGFTFSTYAMNWVRQAPGKGLEWVGRIRTKSN
NYATYYADSVKDRFTISRDDSKNSLYLQMNSLKTEDTAVYYCVETGFAYWDQGTLVTV
SS
The amino acid sequence of Hu147-VH2 is set forth in SEQ ID NO: 28:
EVQLVESGGGLVQPGGSLRLSCAASGFTFNTYAMNWVRQAPGKGLEWVARIRTKSN
NYATYYADSVKDRFTISRDDSKNSLYLQMNSLKTEDTAVYYCVETGFAYWDQGTLVTV
SS
The amino acid sequence of Hu147-VH3 is set forth in SEQ ID NO: 29:
EVQLVESGGGLVQPGGSLRLSCAASGFSFNTYAMNWVRQAPGKGLEWVARIRTKSN
NYATYYADSVKDRFTISRDDSESSLYLQMNSLKTEDTAVYYCVETGFAYWDQGTLVTVS
S.

Note:
The CDR sequences defined by the Kabat numbering scheme are underlined and the order is FR1-CDR1-FR2-CDR2-FR3- CDR3-FR4.

2. Humanization of mab164

The humanization light chain template for the murine antibody mab164 consists of IGLV7-43*01 and IGLJ2*01, IGLV8-61*01 and IGLJ2*01, or IGLV1-40*02 and IGLJ2*01, and the humanization heavy chain template consists of IGHV2-26*01 and IGHJ6*01, or IGHV4-31*02 and IGHJ6*01. The CDRs of the murine antibody mab164 were grafted into its humanization templates, and further, some amino acids of the FR of the humanized antibody were modified with back mutations, wherein back mutations for light chain variable regions include one or more of 36V, 44F, 46G, and 49G, and back mutations for heavy chain variable regions include one or more of 44G, 49G, 27F, 48L, 67L, 71K, 78V, and 80F (the positions of the back mutations in the light and heavy chain variable regions are determined by the Kabat numbering scheme); thus, the humanized antibody heavy chain and the humanized antibody light chain of mab164 were obtained, and the variable region sequences thereof are as follows:

The specific sequences of the variable regions of the mAb164 humanized antibody are as follows:

The amino acid sequence of Hu164-VL7 is set forth in SEQ ID NO: 30:
ETVVTQEPSLTVSPGGTVTLTCRSSIGAVTTSNYANWVQQKPGQAFRGLIGGTSNRAP
WTPARFSGSLLGGKAALTLSGVQPEDEAEYYCALWYSTHYVFGGGTKLTVL
The amino acid sequence of Hu164-VL8 is set forth in SEQ ID NO: 31:
ETVVTQEPSFSVSPGGTVTLTCRSSIGAVTTSNYANWVQQTPGQAFRGLIGGTSNRAP
GVPDRFSGSILGNKAALTITGAQADDESDYYCALWYSTHYVFGGGTKLTVL
The amino acid sequence of Hu164-VL9 is set forth in SEQ ID NO: 32:
ESVVTQPPSVSGAPGQRVTISCRSSIGAVTTSNYANWVQQLPGTAFKGLIGGTSNRAP
GVPDRFSGSKSGTSASLAITGLQAEDEADYYCALWYSTHYVFGGGTKLTVL
The amino acid sequence of Hu164-VH5 is set forth in SEQ ID NO: 33:
EVTLKESGPVLVKPTETLTLTCTVSGFSLSTFGVHWIRQPPGKALEWLAVIWRRGGTD
YNAAFMSRLTISKDTSKSQVVLTMTNMDPVDTATYYCARDGGFDYWGQGTTVTVSS
The amino acid sequence of Hu164-VH6 is set forth in SEQ ID NO: 34:
EVTLKESGPVLVKPTETLTLTCTVSGFSLSTFGVHWIRQPPGKGLEWLGVIWRRGGT
DYNAAFMSRLTISKDTSKSQVVFTMTNMDPVDTATYYCARDGGFDYWGQGTTVTVS
S
The amino acid sequence of Hu164-VH7 is set forth in SEQ ID NO: 35:
EVQLQESGPGLVKPSQTLSLTCTVSGFSISTFGVHWIRQHPGKGLEWIGVIWRRGGT
DYNAAFMSRVTISKDTSKNQVSLKLSSVTAADTAVYYCARDGGFDYWGQGTTVTVSS
The amino acid sequence of Hu164-VH8 is set forth in SEQ ID NO: 36:
EVQLQESGPGLVKPSQTLSLTCTVSGFSISTFGVHWIRQHPGKGLEWLGVIWRRGGT
DYNAAFMSRLTISKDTSKNQVSFKLSSVTAADTAVYYCARDGGFDYWGQGTTVTVSS.

Note:
in the above Hu164 antibody sequences, the CDR sequences are underlined and the order is FR1-CDR1-FR2-CDR2- FR3-CDR3-FR4, wherein the CDR sequences are defined by the Kabat numbering scheme.

3. Humanization of mab95

The humanization light chain templates for the murine antibody mab95 are IGKV3-20*02 and IGKV1-40*01, and the humanization heavy chain template is IGHV1-3*01. The CDRs of the murine antibody mab95 were grafted into its humanization templates, and further, some amino acids of the FR of the humanized antibody were modified with back mutations, wherein back mutations for light chain variable regions include one or more of 45P, 46W, 48Y, 69S, and 70Y, and back mutations for heavy chain variable regions include one or more of 27F, 38K, 481, 67K, 68A, 70L, and 72F (the positions of the back mutations in the light and heavy chain variable regions are determined by the Kabat numbering scheme); light and heavy chain variable regions with different sequences were obtained. Thus, a humanized antibody comprising the CDRs of mab95 was obtained, and the variable region sequences thereof are as follows:

The specific sequences of the variable regions of the mAb95 humanized antibody are as follows:

The amino acid sequence of Hu95-VL1 is set forth in SEQ ID NO: 77:
EIVLTQSPATLSLSPGERATLSCRASSSVSYIHWYQQKPGQAPRPWIYATSNLASGIPARF
SGSGSGTDYTLTISRLEPEDFAVYYCQQWNSNPWTFGGGTKVEIK
The amino acid sequence of Hu95-VL2 is set forth in SEQ ID NO: 78:
EIVLTQSPATLSLSPGERATLSCRASSSVSYIHWYQQKPGQSPRPWIYATSNLASGVPAR
FSGSGSGTSYTLTISRLEPEDFAVYYCQQWNSNPWTFGGGTKVEIK
The amino acid sequence of Hu95-VL3 is set forth in SEQ ID NO: 79:
ESVLTQPPSVSGAPGQRVTISCRASSSVSYIHWYQQLPGTAPKPWIYATSNLASGVPDR
FSGSKSGTSYSLAITGLQAEDEADYYCQQWNSNPWTFGGGTKVEIK
The amino acid sequence of Hu95-VL4 is set forth in SEQ ID NO: 80:
EIVLTQPPSVSGAPGQRVTISCRASSSVSYIHWYQQLPGTSPKPWIYATSNLASGVPDR
FSGSKSGTSYSLAITGLQAEDEADYYCQQWNSNPWTFGGGTKVEIK
The amino acid sequence of Hu95-VH1 is set forth in SEQ ID NO: 81:
EVQLVQSGAEVKKPGASVKVSCKASGFTFTNYDIHWVRQAPGQRLEWMGLVYPYNG
GTAYNQKFKDRVTLTFDTSASTAYMELSSLRSEDTAVYYCARWGLIPGTTSYFDVWGQ
GTTVTVSS
The amino acid sequence of Hu95-VH2 is set forth in SEQ ID NO: 82:
EVQLVQSGAEVKKPGASVKVSCKASGFTFTNYDIHWVRQAPGQRLEWIGLVYPYNG
GTAYNQKFKDRATLTFDTSASTAYMELSSLRSEDTAVYYCARWGLIPGTTSYFDVWGQ
GTTVTVSS
The amino acid sequence of Hu95-VH3 is set forth in SEQ ID NO: 83:
EVQLVQSGAEVKKPGASVKVSCKASGFTFTNYDIHWVKQAPGQRLEWMGLVYPYNG
GTAYNQKFKDKVTLTFDTSASTAYMELSSLRSEDTAVYYCARWGLIPGTTSYFDVWGQ
GTTVTVSS
The amino acid sequence of Hu95-VH4 is set forth in SEQ ID NO: 84:
EVQLVQSGAEVKKPGASVKVSCKASGFTFTNYDIHWVKQAPGQRLEWIGLVYPYNG
GTAYNQKFKDKATLTFDTSASTAYMELSSLRSEDTAVYYCARWGLIPGTTSYFDVWGQ
GTTVTVSS
The amino acid sequence of Hu95-VH5 is set forth in SEQ ID NO: 85:
EVQLVQSGAEVKKPGASVKVSCRASGFTFTNYAIHWVKQAPGQRLEWMGLVYPYTG
GTAYNQKFKDKVTLTFDTSASTAYMELSSLRSEDTAVYYCARWGMIPGTNSYFDVWG
QGTTVTVSS.

Note:
The CDR sequences defined by the Kabat numbering scheme are underlined and the order is FR1-CDR1-FR2-CDR2-FR3- CDR3-FR4.

The CDR regions of Hu95-VH5 were further modified. HCDR1 is set forth in SEQ ID NO: 102 (NYAIH), HCDR2 is set forth in SEQ ID NO: 103 (LVYPYTGGTAYNQKFKD), and HCDR3 is set forth in SEQ ID NO: 104 (WGMIPGTNSYFDV).

### 4. Construction and Expression of Anti-CTGF Humanized Full-Length Antibodies

A primer was designed, and then a VH/VL gene fragment of each humanized antibody was constructed by PCR and subjected to homologous recombination with an expression vector pHr (with a signal peptide and a constant region gene (CH/CL) fragment), thus constructing an expression vector VH-CH-pHr/VL-CL-pHr for a full-length antibody. The constant region of the humanized antibody may be selected from the group consisting of the light chain constant regions of human κ and λ chains, and from the group consisting of the heavy chain constant regions of IgG1, IgG2, IgG3, and IgG4, and variants thereof. Non-limiting examples include optimizing the constant regions of human IgG1, IgG2, and IgG4 to improve antibody function; for example, the half-life of the antibody can be prolonged by M252Y/S254T/T256E (“YTE”) point mutations in the constant region, S228P, F234A, and L235A, and other point mutations in the constant region, and the like.

Exemplary anti-CTGF humanized antibody constant region sequences are as follows:

The heavy chain constant region amino acid sequence is set forth in SEQ ID NO:
37 (w is suffixed to the name of the formed full-length antibody heavy chain):
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSL
SLSPGK
The heavy chain constant region amino acid sequence of the YTE-modified
antibody is set forth in SEQ ID NO: 38 (y is suffixed to the name of the formed
full-length antibody heavy chain):
ASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTC
PPCPAPELLGGPSVFLFPPKPKDTLYITREPEVTCVVVDVSHEDPEVKFNWYVDG
VEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLS
LSPGK
The light chain kappa constant region amino acid sequence of the anti-CTGF
humanized antibody is set forth in SEQ ID NO: 39 (k is suffixed to the name of the
formed full-length antibody heavy chain):
RTVAAPSVFIFPPSDEQLKSGTASVVCLLNNFYPREAKVQWKVDNALQSGNSQE
SVTEQDSKDSTYSLSSTLTLSKADYEKHKVYACEVTHQGLSSPVTKSFNRGEC
The light chain lambda constant region amino acid sequence of the anti-CTGF
humanized antibody is set forth in SEQ ID NO: 40 (1 is suffixed to the name of the
formed full-length antibody heavy chain):
GQPKANPTVTLFPPSSEELQANKATLVCLISDFYPGAVTVAWKADGSPVKAGVE
TTKPSKQSNNKYAASSYLSLTPEQWKSHRSYSCQVTHEGSTVEKTVAPTEC

Full-length humanized antibodies derived from mab147 formed by linking a heavy chain variable region of the humanized antibody to the heavy chain constant region set forth in SEQ ID NO: 37 and linking a light chain variable region of the humanized antibody to the light chain kappa constant region set forth in SEQ ID NO: 39 include:

TABLE 6

| | Hu147-VL1 | Hu147-VL2 | Hu147-VL3 | Hu147-VL4 | Hu147-VL5 |
|---|---|---|---|---|---|
| Hu147-VH1 | Hu147-11wk | Hu147-12wk | Hu147-13wk | Hu147-14wk | Hu147-15wk |
| Hu147-VH2 | Hu147-21wk | Hu147-22wk | Hu147-23wk | Hu147-24wk | Hu147-25wk |
| Hu147-VH3 | Hu147-31wk | Hu147-32wk | Hu147-33wk | Hu147-34wk | Hu147-35wk |

Full-length humanized antibodies derived from mab147 formed by linking a heavy chain variable region of the humanized antibody to the heavy chain constant region YTE variant set forth in SEQ ID NO: 38 and linking a light chain variable region of the humanized antibody to the light chain kappa constant region set forth in SEQ ID NO: 39 include:

TABLE 7

| | Hu147-VL1 | Hu147-VL2 | Hu147-VL3 | Hu147-VL4 | Hu147-VL5 |
|---|---|---|---|---|---|
| Hu147-VH1 | Hu147-11yk | Hu147-12yk | Hu147-13yk | Hu147-14yk | Hu147-15yk |
| Hu147-VH2 | Hu147-21yk | Hu147-22yk | Hu147-23yk | Hu147-24yk | Hu147-25yk |
| Hu147-VH3 | Hu147-31yk | Hu147-32yk | Hu147-33yk | Hu147-34yk | Hu147-35yk |

Full-length humanized antibodies derived from mab164 formed by linking a heavy chain variable region of the humanized antibody to the heavy chain constant region set forth in SEQ ID NO: 37 and linking a light chain variable region of the humanized antibody to the light chain lambda constant region set forth in SEQ ID NO: 40 include:

TABLE 8

| | Hu164-VH5 | Hu164-VH6 | Hu164-VH7 | Hu164-VH8 |
|---|---|---|---|---|
| Hu164-VL7 | Hu164-57wl | Hu164-67wl | Hu164-77wl | Hu164-87wl |
| Hu164-VL8 | Hu164-58wl | Hu164-68wl | Hu164-78wl | Hu164-88wl |
| Hu164-VL9 | Hu164-59wl | Hu164-69wl | Hu164-79wl | Hu164-89wl |

Full-length humanized antibodies derived from mab164 formed by linking a heavy chain variable region of the humanized antibody to the heavy chain constant region YTE variant set forth in SEQ ID NO: 38 and linking a light chain variable region of the humanized antibody to the light chain lambda constant region set forth in SEQ ID NO: 40 include:

TABLE 9

| | Hu164-VH5 | Hu164-VH6 | Hu164-VH7 | Hu164-VH8 |
|---|---|---|---|---|
| Hu164-VL7 | Hu164-57y1 | Hu164-67yl | Hu164-77y1 | Hu164-87yl |
| Hu164-VL8 | Hu164-58y1 | Hu164-68yl | Hu164-78y1 | Hu164-88y1 |
| Hu164-VL9 | Hu164-59y1 | Hu164-69yl | Hu164-79yl | Hu164-89yl |

Full-length humanized antibodies derived from mab95 formed by linking a heavy chain variable region of the humanized antibody to the heavy chain constant region set forth in SEQ ID NO: 37 and linking a light chain variable region of the humanized antibody to the light chain kappa constant region set forth in SEQ ID NO: 39 include: Table 10

TABLE 10

| | Hu95-VH1 | Hu95-VH2 | Hu95-VH3 | Hu95-VH4 | Hu95-VH5 |
|---|---|---|---|---|---|
| Hu95-VL1 | Hu95-11wk | Hu95-21wk | Hu95-31wk | Hu95-41wk | Hu95-51wk |
| Hu95-VL2 | Hu95-12wk | Hu95-22wk | Hu95-32wk | Hu95-42wk | Hu95-52wk |
| Hu95-VL3 | Hu95-13wk | Hu95-23wk | Hu95-33wk | Hu95-43wk | Hu95-53wk |
| Hu95-VL4 | Hu95-14wk | Hu95-24wk | Hu95-34wk | Hu95-44wk | Hu95-54wk |

Full-length humanized antibodies derived from mab95 formed by linking a heavy chain variable region of the humanized antibody to the heavy chain constant region YTE variant set forth in SEQ ID NO: 38 and linking a light chain variable region of the humanized antibody to the light chain kappa constant region set forth in SEQ ID NO: 39 include:

TABLE 11

| | Hu95-VH1 | Hu95-VH2 | Hu95-VH3 | Hu95-VH4 | Hu95-VH5 |
|---|---|---|---|---|---|
| Hu95-VL1 | Hu95-11yk | Hu95-21yk | Hu95-31yk | Hu95-41yk | Hu95-51yk |
| Hu95-VL2 | Hu95-12yk | Hu95-22yk | Hu95-32yk | Hu95-42yk | Hu95-52yk |
| Hu95-VL3 | Hu95-13yk | Hu95-23yk | Hu95-33yk | Hu95-43yk | Hu95-53yk |
| Hu95-VL4 | Hu95-14yk | Hu95-24yk | Hu95-34yk | Hu95-44yk | Hu95-54yk |

Exemplary anti-CTGF antibody full-length amino acid sequences are as follows:

TABLE 12
Light or heavy chains of full-length antibodies

| Light or heavy | |
|---|---|
| chains of full-length | |
| antibodies (SEQ ID NO) | Amino acid sequence |
| Ch147 heavy chain | EVQLVESGGGLVQPEGSLKLSCAASGFSFNTYAMNWVRQAPGKGLEWVARI |
| (SEQ ID NO: 41) | RTKSNNYATYYADSVKDRFTISRDDSESMLYLQMNNLKTEDTAMYYCVETGF |
| | AYWDQGTLVTVSAASTKGPSVFPLAPSSKSTSGGTAALGCLVKDYFPEPV |
| | TVSWNSGALTSGVHTFPAVLQSSGLYSLSSVVTVPSSSLGTQTYICNVN |
| | HKPSNTKVDKKVEPKSCDKTHTCPPCPAPELLGGPSVFLFPPKPKDTLM |
| | ISRTPEVTCVVVDVSHEDPEVKFNWYVDGVEVHNAKTKPREEQYNST |
| | YRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLS |
| | PGK |
| Ch147 light chain | QIVLTQSPAIMSASPGEKVTITCSASSSVSYMHWFQQKPGTSPKLWIYSTSNLA |
| (SEQ ID NO: 42) | SGVPARFSGSGSGTSYSLTISRMEAEDAATYYCQQRSSYPLTFGAGTKLELKR |
| | TVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPV |
| | TKSFNRGEC |
| Full-length heavy | EVQLVESGGGLVQPGGSLRLSCAASGFTFSTYAMNWVRQAPGKGLEWVGR |
| chains of humanized | IRTKSNNYATYYADSVKDRFTISRDDSKNSLYLQMNSLKTEDTAVYYCVETGF |
| antibodies | AYWDQGTLVTVSSASTKGPSVFPLAPSSKSTSGGTAALGCLVKDYFPEPV |
| Hu147-11wk, | TVSWNSGALTSGVHTFPAVLQSSGLYSLSSVVTVPSSSLGTQTYICNVN |
| Hu147-12wk, | HKPSNTKVDKKVEPKSCDKTHTCPPCPAPELLGGPSVFLFPPKPKDTLM |
| Hu147-13wk, | ISRTPEVTCVVVDVSHEDPEVKFNWYVDGVEVHNAKTKPREEQYNST |
| Hu147-14wk, and | YRVVSVLTVLHQDWLNGKEYKCKVSNKALPAPIEKTISKAKGQPREPQ |
| Hu147-15wk | VYTLPPSRDELTKNQVSLTCLVKGFYPSDIAVEWESNGQPENNYKTTPP |
| (SEQ ID NO: 43) | VLDSDGSFFLYSKLTVDKSRWQQGNVFSCSVMHEALHNHYTQKSLSLS |
| | PGK |
| Full-length heavy | EVQLVESGGGLVQPGGSLRLSCAASGFTFNTYAMNWVRQAPGKGLEWVAR |
| chains of humanized | IRTKSNNYATYYADSVKDRFTISRDDSKNSLYLQMNSLKTEDTAVYYCVETGF |
| antibodies | AYWDQGTLVTVSSASTKGPSVFPLAPSSKSTSGGTAALGCLVKDYFPEPV |
| Hu147-21wk, | TVSWNSGALTSGVHTFPAVLQSSGLYSLSSVVTVPSSSLGTQTYICNVN |
| Hu147-22wk, | HKPSNTKVDKKVEPKSCDKTHTCPPCPAPELLGGPSVFLFPPKPKDTLM |
| Hu147-23wk, | ISRTPEVTCVVVDVSHEDPEVKFNWYVDGVEVHNAKTKPREEQYNST |
| Hu147-24wk, and | YRVVSVLTVLHQDWLNGKEYKCKVSNKALPAPIEKTISKAKGQPREPQ |
| Hu147-24wk | VYTLPPSRDELTKNQVSLTCLVKGFYPSDIAVEWESNGQPENNYKTTPP |
| (SEQ ID NO: 44) | VLDSDGSFFLYSKLTVDKSRWQQGNVFSCSVMHEALHNHYTQKSLSLS |
| | PGK |
| Full-length heavy | EVQLVESGGGLVQPGGSLRLSCAASGFSFNTYAMNWVRQAPGKGLEWVARI |
| chains of humanized | RTKSNNYATYYADSVKDRFTISRDDSESSLYLQMNSLKTEDTAVYYCVETGFA |
| antibodies | YWDQGTLVTVSSASTKGPSVFPLAPSSKSTSGGTAALGCLVKDYFPEPVT |
| Hu147-31wk, | VSWNSGALTSGVHTFPAVLQSSGLYSLSSVVTVPSSSLGTQTYICNVNH |
| Hu147-32wk, | KPSNTKVDKKVEPKSCDKTHTCPPCPAPELLGGPSVFLFPPKPKDTLMIS |
| Hu147-33wk, | RTPEVTCVVVDVSHEDPEVKFNWYVDGVEVHNAKTKPREEQYNSTYR |
| Hu147-34wk, and | VVSVLTVLHQDWLNGKEYKCKVSNKALPAPIEKTISKAKGQPREPQVY |
| Hu147-35wk | TLPPSRDELTKNQVSLTCLVKGFYPSDIAVEWESNGQPENNYKTTPPVL |
| (SEQ ID NO: 45) | DSDGSFFLYSKLTVDKSRWQQGNVFSCSVMHEALHNHYTQKSLSLSPG |
| | K |
| Full-length heavy | EVQLVESGGGLVQPGGSLRLSCAASGFTFSTYAMNWVRQAPGKGLEWVGR |
| chains of humanized | IRTKSNNYATYYADSVKDRFTISRDDSKNSLYLQMNSLKTEDTAVYYCVETGF |
| antibodies | AYWDQGTLVTVSSASTKGPSVFPLAPSSKSTSGGTAALGCLVKDYFPEPV |
| Hu147-11yk, | TVSWNSGALTSGVHTFPAVLQSSGLYSLSSVVTVPSSSLGTQTYICNVN |
| Hu147-12yk, | HKPSNTKVDKKVEPKSCDKTHTCPPCPAPELLGGPSVFLFPPKPKDTLYI |
| Hu147-13yk, | TREPEVTCVVVDVSHEDPEVKFNWYVDGVEVHNAKTKPREEQYNSTY |
| Hu147-14yk, and | RVVSVLTVLHQDWLNGKEYKCKVSNKALPAPIEKTISKAKGQPREPQV |
| Hu147-15yk | YTLPPSRDELTKNQVSLTCLVKGFYPSDIAVEWESNGQPENNYKTTPPV |
| (SEQ ID NO: 46) | LDSDGSFFLYSKLTVDKSRWQQGNVFSCSVMHEALHNHYTQKSLSLSP |
| | GK |
| Full-length heavy | EVQLVESGGGLVQPGGSLRLSCAASGFTFNTYAMNWVRQAPGKGLEWVAR |
| chains of humanized | IRTKSNNYATYYADSVKDRFTISRDDSKNSLYLQMNSLKTEDTAVYYCVETGF |
| antibodies | AYWDQGTLVTVSSASTKGPSVFPLAPSSKSTSGGTAALGCLVKDYFPEPV |
| Hu147-21yk, | TVSWNSGALTSGVHTFPAVLQSSGLYSLSSVVTVPSSSLGTQTYICNVN |
| Hu147-22yk, | HKPSNTKVDKKVEPKSCDKTHTCPPCPAPELLGGPSVFLFPPKPKDTLYI |
| Hu147-23yk, | TREPEVTCVVVDVSHEDPEVKFNWYVDGVEVHNAKTKPREEQYNSTY |
| Hu147-24yk, and | RVVSVLTVLHQDWLNGKEYKCKVSNKALPAPIEKTISKAKGQPREPQV |
| Hu147-24yk | YTLPPSRDELTKNQVSLTCLVKGFYPSDIAVEWESNGQPENNYKTTPPV |
| (SEQ ID NO: 47) | LDSDGSFFLYSKLTVDKSRWQQGNVFSCSVMHEALHNHYTQKSLSLSP |
| | GK |
| Full-length heavy | EVQLVESGGGLVQPGGSLRLSCAASGFSFNTYAMNWVRQAPGKGLEWVARI |
| chains of humanized | RTKSNNYATYYADSVKDRFTISRDDSESSLYLQMNSLKTEDTAVYYCVETGFA |
| antibodies | YWDQGTLVTVSSASTKGPSVFPLAPSSKSTSGGTAALGCLVKDYFPEPVT |
| Hu147-31yk, | VSWNSGALTSGVHTFPAVLQSSGLYSLSSVVTVPSSSLGTQTYICNVNH |
| Hu147-32yk, | KPSNTKVDKKVEPKSCDKTHTCPPCPAPELLGGPSVFLFPPKPKDTLYIT |
| Hu147-33yk, | REPEVTCVVVDVSHEDPEVKFNWYVDGVEVHNAKTKPREEQYNSTYR |
| Hu147-34yk, and | VVSVLTVLHQDWLNGKEYKCKVSNKALPAPIEKTISKAKGQPREPQVY |
| Hu147-35yk | TLPPSRDELTKNQVSLTCLVKGFYPSDIAVEWESNGQPENNYKTTPPVL |
| (SEQ ID NO: 48) | DSDGSFFLYSKLTVDKSRWQQGNVFSCSVMHEALHNHYTQKSLSLSPG |
| | K |
| Full-length light | EIVLTQSPATLSLSPGERATLSCSASSSVSYMHWFQQKPGQSPRLLIYSTSNLA |
| chains of humanized | SGIPARFSGSGSGTDYTLTISSLEPEDEAVYYCQQRSSYPLTFGQGTKLEIKRT |
| antibodies | VAAPSVFIFPPSDEQLKSGTASVVCLLNNFYPREAKVQWKVDNALQSG |
| Hu147-11wk, | NSQESVTEQDSKDSTYSLSSTLTLSKADYEKHKVYACEVTHQGLSSPVT |
| Hu147-21wk, and | KSFNRGEC |
| Hu147-31wk | (it is also the full-length light chains of Hu147-11yk, |
| (SEQ ID NO: 49) | H u147-31yk) |
| Full-length light | EIVLTQSPATLSLSPGERATLSCSASSSVSYMHWFQQKPGQSPKLLIYSTSNLA |
| chains of humanized | SGIPARFSGSGSGTDYTLTISSLEPEDFAVYYCQQRSSYPLTFGQGTKLEIKRT |
| antibodies | VAAPSVFIFPPSDEQLKSGTASVVCLLNNFYPREAKVQWKVDNALQSG |
| Hu147-12wk, | NSQESVTEQDSKDSTYSLSSTLTLSKADYEKHKVYACEVTHQGLSSPVT |
| Hu147-22wk, and | KSFNRGEC |
| Hu147-32wk | (it is also the full-length light chains of Hul47-12yk, |
| (SEQ ID NO: 50) | Hul47-22yk, and Hu147-32yk) |
| Full-length light | EIVLTQSPATLSLSPGERATLSCSASSSVSYMHWFQQKPGQSPKLWIYSTSNLA |
| chains of humanized | SGVPARFSGSGSGTDYTLTISSLEPEDFAVYYCQQRSSYPLTFGQGTKLEIKRT |
| antibodies | VAAPSVFIFPPSDEQLKSGTASVVCLLNNFYPREAKVQWKVDNALQSG |
| Hu147-13wk, | NSQESVTEQDSKDSTYSLSSTLTLSKADYEKHKVYACEVTHQGLSSPVT |
| Hu147-23wk, and | KSFNRGEC |
| Hu147-33wk | (it is also the full-length light chains of Hu147-13yk, |
| (SEQ ID NO: 51) | Hu147-23yk, and Hu147-33yk) |
| Full-length light | DIQMTQSPSSLSASVGDRVTITCSASSSVSYMHWFQQKPGKSPKLLIYSTSNL |
| chains of humanized | ASGVPSRFSGSGSGTDYTLTISSLQPEDFATYYCQQRSSYPLTFGQGTKLEIK |
| antibodies | RTVAAPSVFIFPPSDEQLKSGTASVVCLLNNFYPREAKVQWKVDNALQ |
| Hu147-14wk, | SGNSQESVTEQDSKDSTYSLSSTLTLSKADYEKHKVYACEVTHQGLSSP |
| Hu147-24wk, and | VTKSFNRGEC |
| Hu147-34wk | (it is also the full-length light chains of Hu147-14yk, |
| (SEQ ID NO: 52) | Hu147-24yk, and Hu147-34yk) |
| Full-length light | DIQLTQSPSSLSASVGDRVTITCSASSSVSYMHWFQQKPGKSPKLWIYSTSNL |
| chains of humanized | ASGVPSRFSGSGSGTDYTLTISSLQPEDFATYYCQQRSSYPLTFGQGTKLEIK |
| antibodies | RTVAAPSVFIFPPSDEQLKSGTASVVCLLNNFYPREAKVQWKVDNALQ |
| Hu147-15wk, | SGNSQESVTEQDSKDSTYSLSSTLTLSKADYEKHKVYACEVTHQGLSSP |
| Hu147-25wk, and | VTKSFNRGEC |
| Hu147-35wk | (it is also the full-length light chains of Hu147-15yk, |
| (SEQ ID NO: 53) | Hu147-25yk, and Hu147-35yk) |
| Ch164 heavy chain | QVQLKQSGPGLVQPSQSLSITCTVSGFSLTTFGVHWIRQSPGKGLEWLGVI |
| (SEQ ID NO: 54) | WRRGGTDYNAAFMSRLSITKDNSRSHVFFKMTSLQTDDSAIYYCARDGGED |
| | YWGQGTTLTVSSASTKGPSVFPLAPSSKSTSGGTAALGCLVKDYFPEPVT |
| | VSWNSGALTSGVHTFPAVLQSSGLYSLSSVVTVPSSSLGTQTYICNVNH |
| | KPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVL |
| | DSDGSFFLYSKLTVDKSRWQQGNVFSCSVMHEALHNHYTQKSLSLSPG |
| | K |
| Ch164 light chain | QAVVTQESALTTSPGGTVILTCRSSIGAVTTSNYANWVQEKPDHLFTGLIGGT |
| (SEQ ID NO: 55) | SNRAPGVPVRFSGSLIGDKAALTITGAQAEDDAMYFCALWYSTHYVFGGGT |
| | KVTVLGQPKANPTVTLFPPSSEELQANKATLVCLISDFYPGAVTVAWKA |
| | DGSPVKAGVETTKPSKQSNNKYAASSYLSLTPEQWKSHRSYSCQVTHE |
| | GSTVEKTVAPTEC |
| Full-length heavy | EVTLKESGPVLVKPTETLTLTCTVSGFSLSTFGVHWIRQPPGKALEWLAVIW |
| chains of | RRGGTDYNAAFMSRLTISKDTSKSQVVLTMTNMDPVDTATYYCARDGGFDY |
| Hu164-57wl, | WGQGTTVTVSSASTKGPSVFPLAPSSKSTSGGTAALGCLVKDYFPEPVTV |
| Hu164-58wl, and | SWNSGALTSGVHTFPAVLQSSGLYSLSSVVTVPSSSLGTQTYICNVNHKP |
| Hu164-59wl | SNTKVDKKVEPKSCDKTHTCPPCPAPELLGGPSVFLFPPKPKDTLMISRT |
| (SEQ ID NO: 56) | PEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| Full-length heavy | EVTLKESGPVLVKPTETLTLTCTVSGFSLSTFGVHWIRQPPGKGLEWLGVIW |
| chains of | RRGGTDYNAAFMSRLTISKDTSKSQVVFTMTNMDPVDTATYYCARDGGFDY |
| Hu164-67wl, | WGQGTTVTVSSASTKGPSVFPLAPSSKSTSGGTAALGCLVKDYFPEPVTV |
| Hu164-68wl, and | SWNSGALTSGVHTFPAVLQSSGLYSLSSVVTVPSSSLGTQTYICNVNHKP |
| Hu164-69wl | SNTKVDKKVEPKSCDKTHTCPPCPAPELLGGPSVFLFPPKPKDTLMISRT |
| (SEQ ID NO: 57) | PEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| Full-length heavy | EVQLQESGPGLVKPSQTLSLTCTVSGFSISTFGVHWIRQHPGKGLEWIGVIW |
| chains of | RRGGTDYNAAFMSRVTISKDTSKNQVSLKLSSVTAADTAVYYCARDGGFDY |
| Hu164-77wl, | WGQGTTVTVSSASTKGPSVFPLAPSSKSTSGGTAALGCLVKDYFPEPVTV |
| Hu164-78wl, and | SWNSGALTSGVHTFPAVLQSSGLYSLSSVVTVPSSSLGTQTYICNVNHKP |
| Hu164-79wl | SNTKVDKKVEPKSCDKTHTCPPCPAPELLGGPSVFLFPPKPKDTLMISRT |
| (SEQ ID NO: 58) | PEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| Full-length heavy | EVQLQESGPGLVKPSQTLSLTCTVSGFSISTFGVHWIRQHPGKGLEWLGVI |
| chains of | WRRGGTDYNAAFMSRLTISKDTSKNQVSFKLSSVTAADTAVYYCARDGGFD |
| Hu164-87wl, | YWGQGTTVTVSSASTKGPSVFPLAPSSKSTSGGTAALGCLVKDYFPEPVT |
| Hu164-88wl, and | VSWNSGALTSGVHTFPAVLQSSGLYSLSSVVTVPSSSLGTQTYICNVNH |
| Hu164-89wl | KPSNTKVDKKVEPKSCDKTHTCPPCPAPELLGGPSVFLFPPKPKDTLMIS |
| (SEQ ID NO: 59) | RTPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVL |
| | DSDGSFFLYSKLTVDKSRWQQGNVFSCSVMHEALHNHYTQKSLSLSPG |
| | K |
| Full-length heavy | EVTLKESGPVLVKPTETLTLTCTVSGFSLSTFGVHWIRQPPGKALEWLAVIW |
| chains of | RRGGTDYNAAFMSRLTISKDTSKSQVVLTMTNMDPVDTATYYCARDGGFDY |
| Hu164-57yl, | WGQGTTVTVSSASTKGPSVFPLAPSSKSTSGGTAALGCLVKDYFPEPVTV |
| Hu164-58yl, and | SWNSGALTSGVHTFPAVLQSSGLYSLSSVVTVPSSSLGTQTYICNVNHKP |
| Hu164-59yl | SNTKVDKKVEPKSCDKTHTCPPCPAPELLGGPSVFLFPPKPKDTLYITRE |
| (SEQ ID NO: 60) | PEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| Full-length heavy | EVTLKESGPVLVKPTETLTLTCTVSGFSLSTFGVHWIRQPPGKGLEWLGVIW |
| chains of | RRGGTDYNAAFMSRLTISKDTSKSQVVFTMTNMDPVDTATYYCARDGGFDY |
| Hu164-67yl, | WGQGTTVTVSSASTKGPSVFPLAPSSKSTSGGTAALGCLVKDYFPEPVTV |
| Hu164-68yl, and | SWNSGALTSGVHTFPAVLQSSGLYSLSSVVTVPSSSLGTQTYICNVNHKP |
| Hu164-69yl | SNTKVDKKVEPKSCDKTHTCPPCPAPELLGGPSVFLFPPKPKDTLYITRE |
| (SEQ ID NO: 61) | PEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| Full-length heavy | EVQLQESGPGLVKPSQTLSLTCTVSGFSISTFGVHWIRQHPGKGLEWIGVIW |
| chains of | RRGGTDYNAAFMSRVTISKDTSKNQVSLKLSSVTAADTAVYYCARDGGFDY |
| Hu164-77yl, | WGQGTTVTVSSASTKGPSVFPLAPSSKSTSGGTAALGCLVKDYFPEPVTV |
| Hu164-78yl, and | SWNSGALTSGVHTFPAVLQSSGLYSLSSVVTVPSSSLGTQTYICNVNHKP |
| Hu164-79yl | SNTKVDKKVEPKSCDKTHTCPPCPAPELLGGPSVFLFPPKPKDTLYITRE |
| (SEQ ID NO: 62) | PEVTCVVVDVSHEDPEVKFNWYVDGVEVHNAKTKPREEQYNSTYRV |
| | VSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPGK |
| Full-length heavy | EVQLQESGPGLVKPSQTLSLTCTVSGFSISTFGVHWIRQHPGKGLEWLGVI |
| chains of | WRRGGTDYNAAFMSRLTISKDTSKNQVSFKLSSVTAADTAVYYCARDGGFD |
| Hu164-87yl, | YWGQGTTVTVSSASTKGPSVFPLAPSSKSTSGGTAALGCLVKDYFPEPVT |
| Hu164-88yl, and | VSWNSGALTSGVHTFPAVLQSSGLYSLSSVVTVPSSSLGTQTYICNVNH |
| Hu164-89yl | KPSNTKVDKKVEPKSCDKTHTCPPCPAPELLGGPSVFLFPPKPKDTLYIT |
| (SEQ ID NO: 63) | REPEVTCVVVDVSHEDPEVKFNWYVDGVEVHNAKTKPREEQYNSTYR |
| | VVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVL |
| | DSDGSFFLYSKLTVDKSRWQQGNVFSCSVMHEALHNHYTQKSLSLSPG |
| | K |
| Full-length light | ETVVTQEPSLTVSPGGTVTLTCRSSIGAVTTSNYANWVQQKPGQAFRGLIGG |
| chains of | TSNRAPWTPARFSGSLLGGKAALTLSGVQPEDEAEYYCALWYSTHYVFGGG |
| Hu164-57wl, | TKLTVLGQPKANPTVTLFPPSSEELQANKATLVCLISDFYPGAVTVAWKA |
| Hu164-67wl, | DGSPVKAGVETTKPSKQSNNKYAASSYLSLTPEQWKSHRSYSCQVTHE |
| Hu164-77wl, and | GSTVEKTVAPTEC |
| Hu164-87wl | (it is also the full-length light chains of Hu164-57yl, |
| (SEQ ID NO: 64) | Hu164-67yl, Hu164-77yl, and Hu164-87yl) |
| Full-length light | ETVVTQEPSFSVSPGGTVTLTCRSSIGAVTTSNYANWVQQTPGQAFRGLIGG |
| chains of | TSNRAPGVPDRESGSILGNKAALTITGAQADDESDYYCALWYSTHYVFGGGT |
| Hu164-58wl, | KLTVLGQPKANPTVTLFPPSSEELQANKATLVCLISDFYPGAVTVAWKA |
| Hu164-68wl, | DGSPVKAGVETTKPSKQSNNKYAASSYLSLTPEQWKSHRSYSCQVTHE |
| Hu164-78wl, and | GSTVEKTVAPTEC |
| Hu164-88wl | (it is also the full-length light chains of Hu164-58yl, |
| (SEQ ID NO: 65) | Hu164-68yl, Hu164-78yl, and Hu164-88yl) |
| Full-length light | ESVVTQPPSVSGAPGQRVTISCRSSIGAVTTSNYANWVQQLPGTAFKGLIGGT |
| chains of | SNRAPGVPDRFSGSKSGTSASLAITGLQAEDEADYYCALWYSTHYVFGGGTK |
| Hu164-59wl, | LTVLGQPKANPTVTLFPPSSEELQANKATLVCLISDFYPGAVTVAWKAD |
| Hu164-69wl, | GSPVKAGVETTKPSKQSNNKYAASSYLSLTPEQWKSHRSYSCQVTHEG |
| Hu164-79wl, and | STVEKTVAPTEC |
| Hu164-89wl | (it is also the full-length light chains of Hu164-59yl, |
| (SEQ ID NO: 66) | Hu164-69yl, Hul64-79yl, and Hu164-89yl) |
| Ch95 heavy chain | EVLLQQSGPVLVKPGPSVKISCKASGFTFTNYDIHWVKQSHGKSLEWIGLVY |
| (SEQ ID NO: 86) | PYNGGTAYNQKFKDKATLTFDTSSSTAYMELNSLTSEDSAVYYCARWGLIPGT |
| | TSYFDVWGTGTTVTVSSASTKGPSVFPLAPSSKSTSGGTAALGCLVKDYF |
| | PEPVTVSWNSGALTSGVHTFPAVLQSSGLYSLSSVVTVPSSSLGTQTYIC |
| | NVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSL |
| | SLSPGK |
| Ch95 light chain | QIVLSQSPPILSASPGEKVTMTCRASSSVSYIHWYQQKPRSSPKPWIYATSNLA |
| (SEQ ID NO: 87) | SGVPARFSGSGSGTSYSLTISRVEAEDAATYYCQQWNSNPWTFGGGTRLEIK |
| | RTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSP |
| | VTKSFNRGEC |
| Full-length heavy | EVQLVQSGAEVKKPGASVKVSCKASGFTFTNYDIHWVRQAPGQRLEWMGL |
| chains of | VYPYNGGTAYNQKFKDRVTLTFDTSASTAYMELSSLRSEDTAVYYCARWGLIP |
| Hu95-11wk, | GTTSYFDVWGQGTTVTVSSASTKGPSVFPLAPSSKSTSGGTAALGCLVKD |
| Hu95-12wk, | YFPEPVTVSWNSGALTSGVHTFPAVLQSSGLYSLSSVVTVPSSSLGTQTY |
| Hu95-13wk, and | ICNVNHKPSNTKVDKKVEPKSCDKTHTCPPCPAPELLGGPSVFLFPPKP |
| Hu95-14wk | KDTLMISRTPEVTCVVVDVSHEDPEVKFNWYVDGVEVHNAKTKPREE |
| (SEQ ID NO: 88) | QYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQK |
| | SLSLSPGK |
| Full-length heavy | EVQLVQSGAEVKKPGASVKVSCKASGFTFTNYDIHWVRQAPGQRLEWIGLY |
| chains of | YPYNGGTAYNQKFKDRATLTFDTSASTAYMELSSLRSEDTAVYYCARWGLIPG |
| Hu95-21wk, | TTSYFDVWGQGTTVTVSSASTKGPSVFPLAPSSKSTSGGTAALGCLVKDY |
| Hu95-22wk, | FPEPVTVSWNSGALTSGVHTFPAVLQSSGLYSLSSVVTVPSSSLGTQTYI |
| Hu95-23wk, and | CNVNHKPSNTKVDKKVEPKSCDKTHTCPPCPAPELLGGPSVFLFPPKPK |
| Hu95-24wk | DTLMISRTPEVTCVVVDVSHEDPEVKFNWYVDGVEVHNAKTKPREEQ |
| (SEQ ID NO: 89) | YNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKS |
| | LSLSPGK |
| Full-length heavy | EVQLVQSGAEVKKPGASVKVSCKASGFTFTNYDIHWVKQAPGQRLEWMGL |
| chains of | VYPYNGGTAYNQKFKDKVTLTFDTSASTAYMELSSLRSEDTAVYYCARWGLIP |
| Hu95-31wk, | GTTSYFDVWGQGTTVTVSSASTKGPSVFPLAPSSKSTSGGTAALGCLVKD |
| Hu95-32wk, | YFPEPVTVSWNSGALTSGVHTFPAVLQSSGLYSLSSVVTVPSSSLGTQTY |
| Hu95-33wk, and | ICNVNHKPSNTKVDKKVEPKSCDKTHTCPPCPAPELLGGPSVFLFPPKP |
| Hu95-34wk | KDTLMISRTPEVTCVVVDVSHEDPEVKFNWYVDGVEVHNAKTKPREE |
| (SEQ ID NO: 90) | QYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQK |
| | SLSLSPGK |
| Full-length heavy | EVQLVQSGAEVKKPGASVKVSCKASGFTFTNYDIHWVKQAPGQRLEWIGLY |
| chains of | YPYNGGTAYNQKFKDKATLTFDTSASTAYMELSSLRSEDTAVYYCARWGLIPG |
| Hu95-41wk, | TTSYFDVWGQGTTVTVSSASTKGPSVFPLAPSSKSTSGGTAALGCLVKDY |
| Hu95-42wk, | FPEPVTVSWNSGALTSGVHTFPAVLQSSGLYSLSSVVTVPSSSLGTQTYI |
| Hu95-43wk, and | CNVNHKPSNTKVDKKVEPKSCDKTHTCPPCPAPELLGGPSVFLFPPKPK |
| Hu95-44wk | DTLMISRTPEVTCVVVDVSHEDPEVKFNWYVDGVEVHNAKTKPREEQ |
| (SEQ ID NO: 91) | YNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKS |
| | LSLSPGK |
| Full-length heavy | EVQLVQSGAEVKKPGASVKVSCRASGFTFTNYAIHWVKQAPGQRLEWMGL |
| chains of | VYPYTGGTAYNQKFKDKVTLTFDTSASTAYMELSSLRSEDTAVYYCARWGMI |
| Hu95-51wk, | PGTNSYFDVWGQGTTVTVSSASTKGPSVFPLAPSSKSTSGGTAALGCLVK |
| Hu95-52wk, | DYFPEPVTVSWNSGALTSGVHTFPAVLQSSGLYSLSSVVTVPSSSLGTQT |
| Hu95-53wk, and | YICNVNHKPSNTKVDKKVEPKSCDKTHTCPPCPAPELLGGPSVFLFPPK |
| Hu95-54wk | PKDTLMISRTPEVTCVVVDVSHEDPEVKFNWYVDGVEVHNAKTKPRE |
| (SEQ ID NO: 92) | EQYNSTYRVVSVLTVLHQDWLNGKEYKCKVSNKALPAPIEKTISKAKG |
| | QPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQ |
| | KSLSLSPGK |
| Full-length heavy | EVQLVQSGAEVKKPGASVKVSCKASGFTFTNYDIHWVRQAPGQRLEWMGL |
| chains of Hu95-11yk, | VYPYNGGTAYNQKFKDRVTLTFDTSASTAYMELSSLRSEDTAVYYCARWGLIP |
| Hu95-12yk, | GTTSYFDVWGQGTTVTVSSASTKGPSVFPLAPSSKSTSGGTAALGCLVKD |
| Hu95-13yk, and | YFPEPVTVSWNSGALTSGVHTFPAVLQSSGLYSLSSVVTVPSSSLGTQTY |
| Hu95-14yk | ICNVNHKPSNTKVDKKVEPKSCDKTHTCPPCPAPELLGGPSVFLFPPKP |
| (SEQ ID NO: 93) | KDTLYITREPEVTCVVVDVSHEDPEVKFNWYVDGVEVHNAKTKPREE |
| | QYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQK |
| | SLSLSPGK |
| Full-length heavy | EVQLVQSGAEVKKPGASVKVSCKASGFTFTNYDIHWVRQAPGQRLEWIGLY |
| chains of | YPYNGGTAYNQKFKDRATLTFDTSASTAYMELSSLRSEDTAVYYCARWGLIPG |
| Hu95-21yk, | TTSYFDVWGQGTTVTVSSASTKGPSVFPLAPSSKSTSGGTAALGCLVKDY |
| Hu95-22yk, | FPEPVTVSWNSGALTSGVHTFPAVLQSSGLYSLSSVVTVPSSSLGTQTYI |
| Hu95-23yk, and | CNVNHKPSNTKVDKKVEPKSCDKTHTCPPCPAPELLGGPSVFLFPPKPK |
| Hu95-24yk | DTLYITREPEVTCVVVDVSHEDPEVKFNWYVDGVEVHNAKTKPREEQ |
| (SEQ ID NO: 94) | YNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKS |
| | LSLSPGK |
| Full-length heavy | EVQLVQSGAEVKKPGASVKVSCKASGFTFTNYDIHWVKQAPGQRLEWMGL |
| chains of | VYPYNGGTAYNQKFKDKVTLTFDTSASTAYMELSSLRSEDTAVYYCARWGLIP |
| Hu95-31yk, | GTTSYFDVWGQGTTVTVSSASTKGPSVFPLAPSSKSTSGGTAALGCLVKD |
| Hu95-32yk, | YFPEPVTVSWNSGALTSGVHTFPAVLQSSGLYSLSSVVTVPSSSLGTQTY |
| Hu95-33yk, and | ICNVNHKPSNTKVDKKVEPKSCDKTHTCPPCPAPELLGGPSVFLFPPKP |
| Hu95-34yk | KDTLYITREPEVTCVVVDVSHEDPEVKFNWYVDGVEVHNAKTKPREE |
| (SEQ ID NO: 95) | QYNSTYRVVSVLTVLHQDWLNGKEYKCKVSNKALPAPIEKTISKAKGQ |
| | PREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQK |
| | SLSLSPGK |
| Full-length heavy | EVQLVQSGAEVKKPGASVKVSCKASGFTFTNYDIHWVKQAPGQRLEWIGLV |
| chains of | YPYNGGTAYNQKFKDKATLTFDTSASTAYMELSSLRSEDTAVYYCARWGLIPG |
| Hu95-41yk, | TTSYFDVWGQGTTVTVSSASTKGPSVFPLAPSSKSTSGGTAALGCLVKDY |
| Hu95-42yk, | FPEPVTVSWNSGALTSGVHTFPAVLQSSGLYSLSSVVTVPSSSLGTQTYI |
| Hu95-43yk, and | CNVNHKPSNTKVDKKVEPKSCDKTHTCPPCPAPELLGGPSVFLFPPKPK |
| Hu95-44yk | DTLYITREPEVTCVVVDVSHEDPEVKFNWYVDGVEVHNAKTKPREEQ |
| (SEQ ID NO: 96) | YNSTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKS |
| | LSLSPGK |
| Full-length heavy | EVQLVQSGAEVKKPGASVKVSCRASGFTFTNYAIHWVKQAPGQRLEWMGL |
| chains of | VYPYTGGTAYNQKFKDKVTLTFDTSASTAYMELSSLRSEDTAVYYCARWGMI |
| Hu95-51yk, | PGTNSYFDVWGQGTTVTVSSASTKGPSVFPLAPSSKSTSGGTAALGCLVK |
| Hu95-52yk, | DYFPEPVTVSWNSGALTSGVHTFPAVLQSSGLYSLSSVVTVPSSSLGTQT |
| Hu95-53yk, and | YICNVNHKPSNTKVDKKVEPKSCDKTHTCPPCPAPELLGGPSVFLFPPK |
| Hu95-54yk | PKDTLYITREPEVTCVVVDVSHEDPEVKFNWYVDGVEVHNAKTKPRE |
| (SEQ ID NO: 97) | EQYNSTYRVVSVLTVLHQDWLNGKEYKCKVSNKALPAPIEKTISKAKG |
| | QPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQ |
| | KSLSLSPGK |
| Full-length light | EIVLTQSPATLSLSPGERATLSCRASSSVSYIHWYQQKPGQAPRPWIYATSNLA |
| chains of | SGIPARFSGSGSGTDYTLTISRLEPEDFAVYYCQQWNSNPWTFGGGTKVEIK |
| Hu95-11wk, | RTVAAPSVFIFPPSDEQLKSGTASVVCLLNNFYPREAKVQWKVDNALQ |
| Hu95-21wk, | SGNSQESVTEQDSKDSTYSLSSTLTLSKADYEKHKVYACEVTHQGLSSP |
| Hu95-31wk, | VTKSFNRGEC |
| Hu95-41wk, and | (it is also the full-length light chains of Hu95-11yk, |
| Hu95-51wk | Hu95-21yk, Hu95-31yk, Hu95-41yk, and Hu95-51yk) |
| (SEQ ID NO: 98) | |
| Full-length light | EIVLTQSPATLSLSPGERATLSCRASSSVSYIHWYQQKPGQSPRPWIYATSNLA |
| chains of | SGVPARFSGSGSGTSYTLTISRLEPEDFAVYYCQQWNSNPWTFGGGTKVEIK |
| Hu95-12wk, | RTVAAPSVFIFPPSDEQLKSGTASVVCLLNNFYPREAKVQWKVDNALQ |
| Hu95-22wk, | SGNSQESVTEQDSKDSTYSLSSTLTLSKADYEKHKVYACEVTHQGLSSP |
| Hu95-32wk, | VTKSFNRGEC |
| Hu95-42wk, and | (it is also the full-length light chains of Hu95-12yk, |
| Hu95-52wk | Hu95-22yk, Hu95-32yk, Hu95-42yk, and Hu95-52yk) |
| (SEQ ID NO: 99) | |
| Full-length light | ESVLTQPPSVSGAPGQRVTISCRASSSVSYIHWYQQLPGTAPKPWIYATSNLAS |
| chains of | GVPDRFSGSKSGTSYSLAITGLQAEDEADYYCQQWNSNPWTFGGGTKVEIK |
| Hu95-13wk, | RTVAAPSVFIFPPSDEQLKSGTASVVCLLNNFYPREAKVQWKVDNALQ |
| Hu95-23wk, | SGNSQESVTEQDSKDSTYSLSSTLTLSKADYEKHKVYACEVTHQGLSSP |
| Hu95-33wk, | VTKSFNRGEC |
| Hu95-43wk, and | (it is also the full-length light chains of Hu95-13yk, |
| Hu95-53wk | Hu95-23yk, Hu95-33yk, Hu95-43yk, and Hu95-53yk) |
| (SEQ ID NO: 100) | |
| Full-length light | EIVLTQPPSVSGAPGQRVTISCRASSSVSYIHWYQQLPGTSPKPWIYATSNLAS |
| chains of | GVPDRFSGSKSGTSYSLAITGLQAEDEADYYCQQWNSNPWTFGGGTKVEIK |
| Hu95-14wk, | RTVAAPSVFIFPPSDEQLKSGTASVVCLLNNFYPREAKVQWKVDNALQ |
| Hu95-24wk, | SGNSQESVTEQDSKDSTYSLSSTLTLSKADYEKHKVYACEVTHQGLSSP |
| Hu95-34wk, | VTKSFNRGEC |
| Hu95-44wk, and | (it is also the full-length light chains of Hu95-14yk, |
| Hu95-54wk | Hu95-24yk, Hu95-34yk, Hu95-44yk, and Hu95-54yk) |
| (SEQ ID NO: 101) | |

The sequence of the CTGF antibody mAb1 (from CN1829740B under the name pamrevlumab, also known as FG-3019) as a positive control is as follows:

Heavy chain:
(SEQ ID NO: 67)
EGQLVQSGGGLVHPGGSLRLSCAGSGFTFSSYGMHWVRQAPGKGLEWVS
GIGTGGGTYSTDSVKGRFTISRDNAKNSLYLQMNSLRAEDMAVYYCARG
DYYGSGSFFDCWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKRVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK
Light chain:
(SEQ ID NO: 68)
DIQMTQSPSSLSASVGDRVTITCRASQGISSWLAWYQQKPEKAPKSLIY
AASSLQSGVPSRFSGSGSGTDFTLTISSLQPEDFATYYCQQYNSYPPTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

### Examples 1-3. Assays for Binding Activity of CTGF Antibodies

### I. ELISA Assays of CTGF Antibodies Binding to Human CTGF Protein

The binding abilities of anti-human CTGF antibodies were measured by ELISA assays of the antibodies and human CTGF protein and Biacore. A 96-well microplate was directly coated with CTGF recombinant protein. The intensity of the signal after an antibody was added was used to measure the binding activity of the antibody to CTGF.

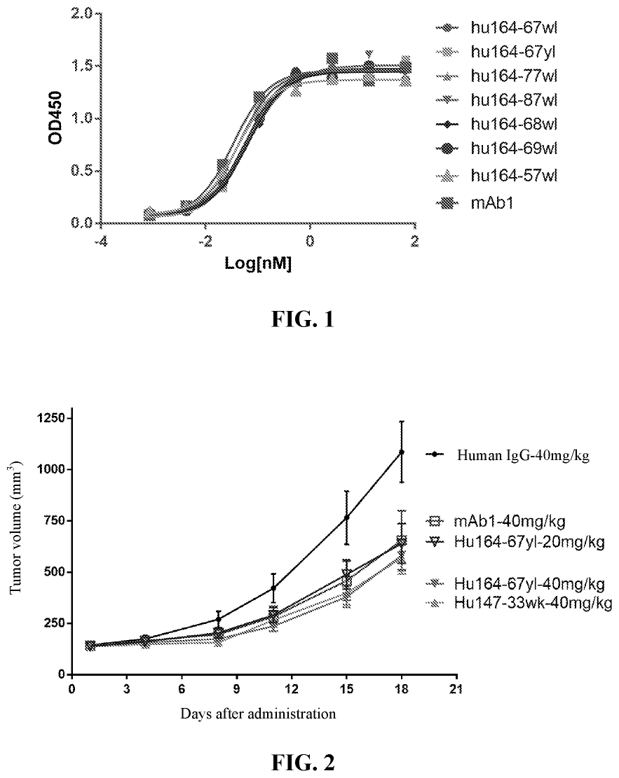

The specific experimental method is as follows: CTGF protein (R&D, Cat No. 9190-CC) was diluted to a concentration of 0.2 g/mL with PBS (Shanghai BasalMedia, Cat No. B320) buffer having a pH of 7.4, and added to a 96-well microplate (Corning, Cat No. CLS3590-100EA) at a volume of 50 μL/well. The plate was left to stand in an incubator at 37° C. for 2 h or at 4° C. overnight (16-18 h). After the liquid was discarded, a PBS-diluted 5% skim milk (BD skim milk, Cat No. 232100) blocking solution was added at 250 μL/well, and the plate was incubated in an incubator at 37° C. for 3 h or left to stand at 4° C. overnight (16-18 h) for blocking. After blocking, the blocking solution was discarded, and the plate was washed 5 times with PBST buffer (PBS containing 0.1% Tween-20, pH 7.4). Then test antibodies (hybridoma purified antibodies or humanized antibodies) that had been diluted with a sample diluent to different concentrations were added at 50 μL/well, and the plate was incubated in a 37° C. incubator for 1 h. After incubation, the plate was washed 3 times with PBST, HRP-labeled goat anti-mouse secondary antibody (Jackson Immuno Research, Cat No. 115-035-003) or goat anti-human secondary antibody (Jackson Immuno Research, Cat No. 109-035-003) that had been diluted with a sample diluent was added at 50 μL/well, and the plate was incubated at 37° C. for 1 h. After the plate was washed 3 times with PBST, TMB chromogenic substrate (KPL, Cat No. 52-00-03) was added at 50 μL/well, and the plate was incubated at room temperature for 5-10 min. The reaction was stopped by adding 1 M H₂SO₄ at 50 μL/well. Absorbance readings were taken at the wavelength of 450 nm on a microplate reader (Molecular Devices, VERSA max). The data were analyzed using GraphPad Prism 5, and the EC50 values of the CTGF antibodies binding to human CTGF protein were calculated. The experimental results are shown in FIG. 1 and Table 13.

TABLE 13
The results of the ELISA assays of CTGF
antibodies binding to human CTGF protein

| | ELISA assay EC50 | | ELISA assay EC50 |
|---|---|---|---|
| Test | of binding to human | Test | of binding to human |
| sample | CTGF protein (nM) | sample | CTGF protein (nM) |
| Hu147-11wk | 0.045 | Hu164-57wl | 0.044 |
| Hu147-12wk | 0.046 | Hu164-67wl | 0.044 |
| Hu147-13wk | 0.043 | Hu164-67yl | 0.060 |
| Hu147-14wk | 0.073 | Hu164-77wl | 0.055 |
| Hu147-15wk | 0.034 | Hu164-87wl | 0.055 |
| Hu147-21wk | 0.067 | Hu164-68wl | 0.069 |
| Hu147-22wk | 0.056 | Hu164-69wl | 0.063 |
| Hu147-23wk | 0.058 | mAb1 | 0.067 |
| Hu147-24wk | 0.048 | Hu95-51yk | 0.080 |
| Hu147-31wk | 0.056 | | |
| Hu147-32wk | 0.063 | | |
| Hu147-33wk | 0.054 | | |
| Hu147-34wk | 0.134 | | |

The experimental results show that all the humanized full-length anti-CTGF antibodies of the present disclosure have a good binding effect on human CTGF protein.

### II. ELISA Assays of CTGF Antibodies Binding to Cynomolgus Monkey CTGF Protein

The binding abilities of anti-monkey CTGF antibodies were measured by ELISA assays of the antibodies and monkey CTGF protein. A 96-well microplate was directly coated with anti-mFc protein. Subsequently, Fc-tagged cyno-CTGF protein was allowed to bind to the plate. Then an antibody was added. The intensity of the signal after the addition was used to measure the binding activity of the antibody to cyno-CTGF. The specific experimental method is as follows:

Anti-mFc protein (Sigma, Cat No. M4280) was diluted to a concentration of 2 g/mL with PBS (Shanghai BasalMedia, Cat No. B320) buffer having a pH of 7.4, and added to a 96-well microplate (Corning, Cat No. CLS3590-100EA) at a volume of 50 μL/well. The plate was left to stand in an incubator at 37° C. for 2 h. After the liquid was discarded, a PBS-diluted 5% skim milk (BD skim milk, Cat No. 232100) blocking solution was added at 250 μL/well, and the plate was incubated in an incubator at 37° C. for 3 h or left to stand at 4° C. overnight (16-18 h) for blocking. After blocking, the blocking solution was discarded, and the plate was washed 3 times with PBST buffer (PBS containing 0.1% Tween-20, pH 7.4). Then 50 μL of 1 μg/mL cyno-CTGF-mFc protein was added to each well. The plate was incubated in a 37° C. incubator for 1 h and then washed 3 times with PBST buffer (PBS containing 0.1% Tween-20, pH 7.4). Then test antibodies (hybridoma purified antibodies or humanized antibodies) that had been diluted with a sample diluent to different concentrations were added at 50 μL/well, and the plate was incubated in a 37° C. incubator for 1 h. After incubation, the plate was washed 3 times with PBST, HRP-labeled goat anti-mouse secondary antibody (Jackson Immuno Research, Cat No. 115-035-003) or goat anti-human secondary antibody (Jackson Immuno Research, Cat No. 109-035-003) that had been diluted with a sample diluent was added at 50 μL/well, and the plate was incubated at 37° C. for 1 h. After the plate was washed 5 times with PBST, TMB chromogenic substrate (KPL, Cat No. 52-00-03) was added at 50 μL/well, and the plate was incubated at room temperature for 5-10 min. The reaction was stopped by adding 1 M H₂SO₄ at 50 μL/well. Absorbance readings were taken at the wavelength of 450 nm on a microplate reader (Molecular Devices, VERSA max). The data were analyzed using GraphPad Prism 5, and the EC50 values of the CTGF antibodies binding to cynomolgus monkey CTGF were calculated. The experimental results are shown in Table 14.

TABLE 14
The results of the ELISA assays of CTGF antibodies
binding to cynomolgus monkey CTGF protein

| | EC50 | | EC50 | | EC50 |
|---|---|---|---|---|---|
| Test sample | (nM) | Test sample | (nM) | Test sample | (nM) |
| Hu147-11wk | 0.052 | Hu147-23wk | 0.063 | Hu164-57wl | 0.029 |
| Hu147-12wk | 0.062 | Hu147-24wk | 0.080 | Hu164-67wl | 0.028 |
| Hu147-13wk | 0.043 | Hu147-31wk | 0.070 | Hu164-77wl | 0.038 |
| Hu147-14wk | 0.077 | Hu147-32wk | 0.079 | Hu164-87wl | 0.034 |
| Hu147-15wk | 0.053 | Hu147-33wk | 0.044 | Hu164-68wl | 0.036 |
| Hu147-21wk | 0.053 | Hu147-34wk | 0.144 | Hu164-69wl | 0.032 |
| Hu147-22wk | 0.073 | Hu95-51yk | 0.059 | mAb1 | 0.086 |

The experimental results show that all the humanized anti-CTGF antibodies of the present disclosure have a good binding effect on monkey CTGF protein.

### III. ELISA Assays of CTGF Antibodies and Mouse CTGF Protein

The binding abilities of anti-mouse CTGF antibodies were measured by ELISA assays of the antibodies and mouse CTGF protein. A 96-well microplate was directly coated with mouse-CTGF fusion protein. The intensity of the signal after an antibody was added was used to measure the binding activity of the antibody to mouse-CTGF.

The specific experimental method is as follows: Mouse CTGF-his protein was diluted to a concentration of 0.5 μg/mL with PBS (Shanghai BasalMedia, Cat No. B320) buffer having a pH of 7.4, and added to a 96-well microplate (Corning, Cat No. CLS3590-100EA) at a volume of 50 μL/well. The plate was left to stand in an incubator at 37° C. for 2 h. After the liquid was discarded, a PBS-diluted 5% skim milk (BD skim milk, Cat No. 232100) blocking solution was added at 250 μL/well, and the plate was incubated in an incubator at 37° C. for 3 h or left to stand at 4° C. overnight (16-18 h) for blocking. After blocking, the blocking solution was discarded, and the plate was washed 3 times with PBST buffer (pH 7.4 PBS containing 0.1% Tween-20). Then test antibodies (hybridoma purified antibodies or humanized antibodies) that had been diluted with a sample diluent to different concentrations were added at 50 μL/well, and the plate was incubated in a 37° C. incubator for 1 h. After incubation, the plate was washed 3 times with PBST, HRP-labeled goat anti-mouse secondary antibody (Jackson Immuno Research, Cat No. 115-035-003) or goat anti-human secondary antibody (Jackson Immuno Research, Cat No. 109-035-003) that had been diluted with a sample diluent was added at 50 μL/well, and the plate was incubated at 37° C. for 1 h. After the plate was washed 5 times with PBST, TMB chromogenic substrate (KPL, Cat No. 52-00-03) was added at 50 μL/well, and the plate was incubated at room temperature for 5-10 min. The reaction was stopped by adding 1 M H₂SO₄ at 50 μL/well. Absorbance readings were taken at the wavelength of 450 nm on a microplate reader (Molecular Devices, VERSA max). The data were analyzed using GraphPad Prism 5, and the EC50 values of the CTGF antibodies binding to mouse CTGF were calculated. The experimental results are shown in Table 15.

TABLE 15
The results of the ELISA assays of CTGF
antibodies binding to mouse CTGF protein

| | EC50 | | EC50 | | EC50 |
|---|---|---|---|---|---|
| Test sample | (nM) | Test sample | (nM) | Test sample | (nM) |
| Hu147-11wk | 0.228 | Hu147-23wk | 0.200 | Hu164-57wl | 0.041 |
| Hu147-12wk | 0.249 | Hu147-24wk | 0.263 | Hu164-67wl | 0.049 |
| Hu147-13wk | 0.127 | Hu147-31wk | 0.320 | Hu164-77wl | 0.058 |
| Hu147-14wk | 0.431 | Hu147-32wk | 0.349 | Hu164-87wl | 0.067 |
| Hu147-15wk | 0.159 | Hu147-33wk | 0.258 | Hu164-68wl | 0.065 |
| Hu147-21wk | 0.247 | Hu147-34wk | 0.664 | Hu164-69wl | 0.059 |
| Hu147-22wk | 0.336 | | | mAb1 | 0.077 |

The experimental results show that all the humanized full-length anti-CTGF antibodies of the present disclosure have a good binding effect on mouse CTGF protein.

### Examples 1-4. Function-Blocking Experiment with CTGF Antibodies

In this experiment, the activity of the test humanized anti-CTGF antibodies blocking human TGF-β1 function was measured using a Biacore instrument.

The antigen human CTGF was firstly fixed onto a CM5 biosensor chip (Cat. #BR-1005-30, GE) by amino coupling at a level of 1500 RU. Then high-concentration CTGF antibody (150 μg/ml) was allowed to flow over the surface of the chip for 150 s, and 50 nM TGF-β1 protein was injected for 120 s. The reaction signals were detected in real time using a Biacore T200 instrument to obtain an association and dissociation curve. After dissociation was complete in each test cycle, the biosensor chip was washed thoroughly and regenerated with a regeneration buffer.

The data obtained from the experiment were statistically analyzed using GE Biacore T200 Evaluation version 3.0 software.

Blocking efficiency=[1−(sample response value/blank control response value)]×100%. The specific results are shown in Table 16.

TABLE 16
The results of the function-blocking
experiment with CTGF antibodies

| | Percent function | | Percent function |
|---|---|---|---|
| Test sample | blocking (%) | Test sample | blocking (%) |
| Hu147-11wk | 50.8% | Hu147-24wk | 52.8% |
| Hu147-12wk | 51.6% | Hu147-31wk | 55.0% |
| Hu147-13wk | 53.3% | Hu147-32wk | 55.2% |
| Hu147-14wk | 50.9% | Hu147-33wk | 57.3% |
| Hu147-15wk | 52.3% | Hu147-34wk | 56.2% |
| Hu147-21wk | 51.6% | Hu164-67wl | 55.5% |
| Hu147-22wk | 52.3% | Hu164-67yl | 53.9% |
| Hu147-23wk | 54.6% | mAb1 | 41.3% |

The experimental results show that all the humanized anti-CTGF antibodies of the present disclosure have high function-blocking activity.

### Biacore Assays for Affinities of Anti-CTGF Antibodies

In the assays, the affinities of the test humanized anti-CTGF antibodies for human CTGF and mouse CTGF were measured using a Biacore instrument.

A Protein A biosensor chip (Cat. #29127556, GE) was allowed to affinity capture a certain amount of a test antibody, and then a certain concentration of human or mouse CTGF antigen was allowed to flow over the surface of the chip. The reaction signals were detected in real time using a Biacore instrument to obtain an association and dissociation curve. After each cycle of dissociation was complete, the biochip was washed thoroughly and regenerated with a pH 1.5 glycine-hydrochloric acid regeneration solution (Cat. #BR-1003-54, GE). The running buffer for the experiment was 1×THBS-EP buffer (Cat. #3BR-1001-88, GE).

The data obtained from the experiment were fitted using GE Biacore T200 Evaluation version 3.0 software with the (1:1) Langmuir model to obtain affinity values, which are shown in detail in Tables 17 and 18.

TABLE 17
The reaction affinities of CTGF
antibodies for human CTGF protein

| Antibody | ka (1/Ms) | kd (1/s) | KD (M) |
|---|---|---|---|
| Ch147 | 1.49E+07 | 6.93E−03 | 4.66E−10 |
| Ch164 | 6.62E+07 | 8.83E−03 | 1.33E−10 |
| Ch95 | 7.85E+06 | 3.02E−02 | 3.85E−09 |
| Hu147-11wk | 2.98E+07 | 3.36E−02 | 1.13E−09 |
| Hu147-12wk | 3.41E+07 | 3.30E−02 | 9.69E−10 |
| Hu147-13wk | 2.97E+07 | 2.40E−02 | 8.08E−10 |
| Hu147-14wk | 4.89E+07 | 5.54E−02 | 1.13E−09 |
| Hu147-21wk | 1.66E+07 | 1.62E−02 | 9.74E−10 |
| Hu147-22wk | 1.40E+07 | 1.42E−02 | 1.01E−09 |
| Hu147-23wk | 1.37E+07 | 1.12E−02 | 8.18E−10 |
| Hu147-31wk | 1.78E+07 | 1.59E−02 | 8.94E−10 |
| Hu147-32wk | 1.46E+07 | 1.33E−02 | 9.10E−10 |
| Hu147-33wk | 1.45E+07 | 1.04E−02 | 7.13E−10 |
| Hu147-34wk | 1.47E+07 | 1.55E−02 | 1.05E−09 |
| Hu164-57wl | 4.26E+07 | 2.94E−03 | 6.91E−11 |
| Hu164-67wl | 5.17E+07 | 1.96E−03 | 3.79E−11 |
| Hu164-67yl | 3.43E+07 | 2.27E−03 | 6.62E−11 |
| Hu164-77wl | 5.15E+07 | 1.76E−02 | 3.43E−10 |
| Hu164-87wl | 1.05E+08 | 2.04E−02 | 1.94E−10 |
| Hu164-68wl | 3.84E+07 | 1.16E−02 | 3.02E−10 |
| Hu164-69wl | 8.71E+07 | 2.80E−02 | 3.22E−10 |
| Hu95-21wk | 4.09E+06 | 3.03E−02 | 7.39E−09 |
| Hu95-31wk | 5.59E+06 | 3.26E−02 | 5.84E−09 |
| Hu95-41wk | 7.70E+06 | 4.08E−02 | 5.29E−09 |
| Hu95-42wk | 3.32E+06 | 1.91E−02 | 5.74E−09 |
| Hu95-51yk | 2.84E+07 | 1.02E−03 | 3.58E−11 |

TABLE 18
The reaction affinities of CTGF
antibodies for mouse CTGF protein

| Antibody | ka (1/Ms) | kd (1/s) | KD (M) |
|---|---|---|---|
| Ch147 | 3.12E+07 | 3.84E−02 | 1.23E−09 |
| Ch164 | 1.23E+08 | 1.01E−02 | 8.22E−11 |
| Ch95 | 1.38E+07 | 2.35E−02 | 1.71E−09 |
| Hu147-11wk | 1.20E+08 | 3.74E−01 | 3.11E−09 |
| Hu147-12wk | 3.75E+07 | 1.16E−01 | 3.09E−09 |
| Hu147-13wk | 5.73E+07 | 1.46E−01 | 2.55E−09 |
| Hu147-23wk | 8.18E+07 | 1.73E−01 | 2.12E−09 |
| Hu147-31wk | 3.83E+07 | 1.07E−02 | 2.79E−09 |
| Hu147-32wk | 1.37E+08 | 3.43E−01 | 2.49E−09 |
| Hu147-33wk | 9.01E+07 | 1.74E−01 | 1.93E−09 |
| Hu164-57wl | 7.35E+07 | 2.37E−03 | 3.22E−11 |
| Hu164-67wl | 6.36E+07 | 1.17E−03 | 1.84E−11 |
| Hu164-67yl | 5.34E+07 | 1.94E−03 | 3.63E−11 |
| Hu164-77wl | 5.98E+07 | 8.83E−03 | 1.48E−10 |
| Hu164-87wl | 5.24E+07 | 4.40E−03 | 8.40E−11 |
| Hu164-68wl | 5.70E+07 | 7.60E−03 | 1.33E−10 |
| Hu164-69wl | 6.17E+07 | 7.74E−03 | 1.26E−10 |
| Hu95-21wk | 5.29E+06 | 2.11E−02 | 3.99E−09 |
| Hu95-31wk | 7.84E+06 | 2.25E−02 | 2.87E−09 |
| Hu95-41wk | 1.39E+07 | 3.39E−02 | 2.44E−09 |
| Hu95-42wk | 5.66E+06 | 1.37E−02 | 2.42E−09 |
| Hu95-51yk | 2.56E+07 | 5.16E−04 | 2.02E−11 |

The experimental results show that the chimeric antibodies Ch147 and Ch164 derived from the murine antibodies mAb147 and mAb164 and the humanized anti-CTGF antibodies can all bind to human CTGF and mouse CTGF with high affinity; all the substitutions with various human antibody germline FR regions and back mutations did not affect the affinity of the humanized antibodies, and some of the modifications could even enhance the affinity of antibodies for antigens.

### Examples 1-5. Inhibition of TGFβ-Induced In Vitro Migration of C2C12 Cells by CTGF Antibodies

C2C12 cells (mouse myoblasts, Cell Bank, Chinese Academy of Sciences, #GNM26) were digested with 0.25% pancreatin (Gibco, #25200-072), centrifuged, and then resuspended in a serum-free DMEM medium (Gibco, #10564-029). Cell-antibody mixtures and TGFβ1-antibody mixtures were prepared with a serum-free DMEM medium; the cell density was 2×10⁵/mL, the concentration of recombinant human TGFβ1 (Cell signaling Technology, #8915LC) was 10 ng/mL, and the concentrations of test antibodies were all 30 μg/mL.

The upper lid and filter membrane of a ChemoTx® Disposable Chemotaxis System (Neuro Probe, #106-8) were removed, and the TGFβ-antibody mixtures were added to the lower chamber at 30 μL/well, each group in quadruplicate. The filter membrane was put back on, and the cell-antibody mixtures were added to the membrane at 50 μL/well, each group in quadruplicate. The upper lid was put back on, and the system was placed into an incubator (37° C., 5% CO₂).

After 48 h of incubation, a clean paper towel was used to soak up the liquid on the filter membrane. The filter membrane was removed, 10 μL of pre-cooled 0.25% pancreatin was added to each well in the lower chamber, and the filter membrane was put back on. After 5 min of digestion, the system was centrifuged at 1000 rpm for 1 min. The filter membrane was removed, and 20 μL of Cell Titer-Glo solution (Promega, #G7573) was added to each well in the lower chamber. The system was incubated at room temperature for 10 min. The liquid was transferred to a 384-well white-bottom plate (Thermo Scientific, #267462), and the plate was read by chemiluminescence using a microplate reader (BMG, #PheraStar). The data were analyzed and processed using Graphpad Prism 6.

Inhibition rate=[(TGFβ mean value−sample group mean value)/(TGFβgroup mean value−untreated group mean value)]×100%

TABLE 19
The results of the inhibition of in vitro
migration of C2C12 cells by CTGF antibodies

| | Percent inhibition of in vitro |
|---|---|
| Test sample | migration of C2C12 cells (%) |
| Hu147-11wk | 72.2% |
| Hu147-33wk | 63.8% |
| Hu164-57wl | 75.4% |
| Hu164-67wl | 78.8% |
| Hu164-67yl | 76.5% |
| Hu164-77wl | 72.8% |
| Hu164-87wl | 76.4% |
| Hu164-68wl | 76.3% |
| Hu164-69wl | 74.2% |
| Hu95-51yk | 80.0% |
| mAb1 | 63.0% |

The results show that the humanized anti-CTGF antibodies of the present disclosure can inhibit the in vitro migration ability of C2C12 cells after TGFβ1 induction and exhibited greater inhibition of the migration ability of C2C12 cells than the positive control antibody mAb1.

### Examples 1-6. Inhibition of TGFβ-Induced In Vitro Migration of PANC1 Cells by CTGF Antibodies

PANC-1 cells (human pancreatic cancer cells, ATCC, #CRL-1469) were digested with 0.25% pancreatin (Gibco, #25200-072), centrifuged, and then resuspended in a DMEM medium (Gibco, #10564-029) containing 0.1% fetal bovine serum (Gibco, #10099-141). Cell-antibody mixtures and TGFβ-antibody mixtures were prepared with a DMEM medium containing 0.1% fetal bovine serum; the cell density was 4×10⁵/mL, the concentration of recombinant human TGFβ (Cell signaling Technology, #8915LC) was 10 ng/mL, and the concentrations of test antibodies were all g/mL.

The upper lid and filter membrane of a ChemoTx® Disposable Chemotaxis System (Neuro Probe, #106-8) were removed, and the TGFβ-antibody mixtures were added to the lower chamber at 30 μL/well, each group in quadruplicate. The filter membrane was put back on, and the cell-antibody mixtures were added to the membrane at 50 μL/well, each group in quadruplicate. The upper lid was put back on, and the system was placed into an incubator (37° C., 5% CO₂).

After 48 h of incubation, a clean paper towel was used to soak up the liquid on the filter membrane. The filter membrane was removed, 10 μL of pre-cooled 0.25% pancreatin was added to each well in the lower chamber, and the filter membrane was put back on. After 5 min of digestion, the system was centrifuged at 1000 rpm for 1 min. Cells that settled to the bottom of the plate were counted under an inverted microscope (Leica, #DMIL LED). The data were analyzed and processed using Graphpad Prism 6.

Inhibition rate=[(TGFβ mean value−sample group mean value)/(TGFβgroup mean value−untreated group mean value)]×100%

TABLE 20
The results of the inhibition of in vitro
migration of PANC1 cells by CTGF antibodies

| | Percent inhibition of | | Percent inhibition of |
|---|---|---|---|
| Test | in vitro migration of | Test | in vitro migration of |
| sample | PANC1 cells (%) | sample | PANC1 cells (%) |
| Hu147-11wk | 73.9% | Hu164-77wl | 74.3% |
| Hu147-33wk | 77.0% | Hu164-87wl | 82.1% |
| Hu164-57wl | 81.2% | Hu164-68wl | 76.9% |
| Hu164-67wl | 75.7% | Hu164-69wl | 76.9% |
| Hu164-67yl | 78.0% | mAb1 | 71.7% |
| Hu95-51yk | 80.0% | | |

The results show that all the humanized anti-CTGF antibodies of the present disclosure can inhibit the in vitro migration ability of PANC1 cells after TGFβ1 induction.

### Example 1-7. Inhibition of In Vitro Proliferation of PANC1 in Soft Agar by CTGF Antibodies

Deionized water was added to agar (BD, #214220), and the mixture was heated to make a 1.4% gel solution. A 2×DMEM medium (Thermo, #12100046) was mixed with the gel solution in a ratio of 1:1 (v/v), and 500 μL of the mixture was added to each well of a 24-well plate (Costar, #3524) and left at 4° C. to coagulate.

PANC-1 cells (human pancreatic cancer cells, ATCC, #CRL-1469) were digested with 0.25% pancreatin (Gibco, #25200-072) and centrifuged, and then the cell density was adjusted to 5×10⁴ cells/mL with a DMEM medium (GE, #SH30243.01) containing 4% fetal bovine serum (Gibco, #10099-141). 2 mg/mL antibodies were prepared with a 2×DMEM medium. The cells, antibodies, and 1.4% gel solution were mixed in a ratio of 2:1:1 (v/v), and 200 μL of the mixture was added to each well of a 24-well plate in which resolving gel had been added. After the upper gel coagulated, 200 μL of a DMEM medium containing 2% fetal bovine serum was added to the 24-well plate. After 28 days of culture in an incubator (37° C., 5% CO₂), photographic records were made using a high-content analysis system (Molecular Devices, ImageXpress), and the area of cell clones formed was analyzed. Inhibition rate=(1-sample group mean value/untreated group mean value)×100%. The experimental results are shown in Table 21.

TABLE 21
The results of the inhibition of in vitro proliferation
of PANC1 cells in soft agar by CTGF antibodies

| | Percent inhibition of in vitro |
|---|---|
| Test sample | proliferation of PANC1 cells (%) |
| Hu147-11wk | 23.76% |
| Hu147-33wk | 22.30% |
| Hu164-67wl | 24.2% |
| Hu164-67yl | 21.7% |
| Hu95-51yk | 20.24% |

The results show that the humanized antibodies from the mAb147 and mAb164 clones have significant inhibitory effects on the in vitro proliferation of human pancreatic cancer cells (PANC-1 cells).

### Examples 1-8. Biological Activity of CTGF Antibodies in Animals

### I. Inhibition of Bleomycin-Induced Mouse Pulmonary Fibrosis by CTGF Antibodies

Experimental method: 40 mice were randomly divided by body weight into the following 4 groups: normal control group (PBS, ip, qod), mAb1 group (10 mg/kg, ip, qod), Hu164-67wl (10 mg/kg, ip, qod) group, and Hu164-67yl (10 mg/kg, ip, qod) group. Each group included 10 mice. The specific grouping is shown in Table 21 below. On the 1st day of the experiment, corresponding solvents and antibodies (10 mL/kg, the normal control and model groups were given PBS) were intraperitoneally injected according to body weight. After 2 h, modeling was performed by bleomycin nebulization (nebulization was performed at 50 mg/8 mL for 30 min and held for 5 min). Then the solvents or antibodies were intraperitoneally injected once every other day according to the groups. The experimental period was 21 days, and the intraperitoneal administration was performed 11 times in total. On the 22nd day of the experiment, mice were anesthetized by an intraperitoneal injection of 1% pentobarbital sodium (10 mL/kg) and then fixed onto an operation table in a supine position. A 1 cm skin incision was made along the median line of the neck, and the lower end of the incision reached the entrance of the chest. The subcutaneous connective tissues and muscles were bluntly separated with forceps to expose the trachea. The connective tissues on both sides of the trachea and between the trachea and the esophagus were separated to isolate the trachea free. A venous indwelling needle was inserted and fixed by ligation with a surgical suture at a place where the cannula entered the trachea. 0.8 mL of PBS was drawn with a 1 mL syringe, and the syringe was connected to the inlet end of the venous indwelling needle. PBS was slowly injected into the trachea, stayed for 30 s, and then slowly withdrawn. An opalescent foamy liquid could be observed during the withdrawal. The lavage was repeated three times, and the lavage fluid was transferred to an EP tube. Then 0.5 mL of PBS was drawn, and the above steps were repeated. The two lavage fluids were mixed and centrifuged at 1500 rpm for 5 min, and the supernatant was stored at −20° C. before analysis. The BALF supernatant was centrifuged again at 10,000 rpm for 10 min, and then the supernatant was taken and tested for soluble collagen (kit: Biocolor, Lot No. AA883).

TABLE 22
Grouping and dosing regimen

| | Number | | | Route of | |
|---|---|---|---|---|---|
| | of | | Dose | adminis- | Frequency of |
| Group | animals | Drug | (mg/mL) | tration | administration |
| 1 | 10 | PBS | — | i.p. | Once every |
| | | | | | other day |
| 2 | 10 | mAb1 | 10 | i.p. | Once every |
| | | | | | other day |
| 3 | 10 | Hu164-67wl | 10 | i.p. | Once every |
| | | | | | other day |
| 4 | 10 | Hu164-67yl | 10 | i.p. | Once every |
| | | | | | other day |

Excel statistical software was used: the average values were calculated as avg (average); SD (standard diviation) values were calculated as STDEV (standard diviation); SEM (standard error of mean) values were calculated as STDEV/SQRT (square root) (number of animals per group); GraphPad Prism software was used for plotting, and the data were statistically analyzed using two-way ANOVA or one-way ANOVA. The experimental results are shown in Table 23.

TABLE 23
The experimental results of the inhibition of bleomycin-
induced mouse pulmonary fibrosis by CTGF antibodies

| | mAb1 | Hu164-67wl | Hu164-67yl |
|---|---|---|---|
| Drug | (10 mpk) | (10 mpk) | (10 mpk) |
| Increased | 53.2% | 72.8% | 80.7% |
| collagen | | (p < 0.05) | (p < 0.05) |
| inhibition rate | | | |

The results show that Hu164-67wl or Hu164-67yl exhibited a strong inhibitory effect on pulmonary fibrosis in mice.

### II. Inhibition of Subcutaneous Xenograft Tumors of Su86.86 Human Pancreatic Cancer Cells by CTGF Antibodies

Experimental method: 200 μL of SU86.86 cells (ATCC, CRL-1837, 3.0×10⁶ cells) were inoculated subcutaneously into the right flank of Nu/Nu nude mice. Eleven days after the inoculation, when the tumor volume reached about 140 mm³, mice that were too heavy or mice with tumors that were too big or too small were excluded. Mice were randomly divided by tumor volume into 5 groups of 10, and administration was started on that day. Antibodies were administered intraperitoneally twice a week for 18 days in total. The tumor volume and body weight were measured 1-2 times a week, and the data were recorded. The grouping and dosing regimen are shown in Table 24.

TABLE 24
Grouping and dosing regimen

| | | | | Route of | Frequency of |
|---|---|---|---|---|---|
| | Number of | | Dose | adminis- | adminis- |
| Group | animals | Drug | (mg/kg) | tration | tration |
| 1 | 10 | Human IgG | 40 | i.p | Twice a week |
| 2 | 10 | mAb1 | 40 | i.p | Twice a week |
| 3 | 10 | Hu164-67yl | 40 | i.p | Twice a week |
| 4 | 10 | Hu164-67yl | 20 | i.p | Twice a week |
| 5 | 10 | Hu147-33wk | 40 | i.p | Twice a week |

Data were recorded using Excel statistical software: the average values were calculated as avg; the SD values were calculated as STDEV; the SEM values were calculated as STDEV/SQRT (number of animals per group); GraphPad Prism software was used for plotting, and statistical analysis of the data was performed using Two-way ANOVA or One-way ANOVA.

Tumor volume (V) was calculated as: V=½×L_{long}×Lₛₕₒᵣₜ²

Relative tumor proliferation rate T/C (%)=(Tₜ−T₀)/(Cₜ−C₀)×100, where Tₜ and Cₜ are the tumor volumes of the treatment group and control group at the end of the experiment; T₀ and C₀ are the tumor volumes at the beginning of the experiment.

Tumor growth inhibition TGI (%)=1−T/C(%).

Experimental Results:

During days 1-18 of the experiment (DO stands for the start of the experiment and D18 for day 18), administration was performed by subcutaneous injection twice a week, and the inhibitory effects of CTGF antibodies on SU86.86 tumor growth were measured. The experimental results are shown in Table 24 and FIG. 2. Compared to the blank control group of isotype human IgG, the mAb1-40 mg/kg, Hu164-67yl-40 mg/kg, Hu164-67yl-20 mg/kg, and Hu147-33wk (40 mg/kg) groups exhibited 45%, 53%, 47%, and 54% tumor growth inhibition, respectively. The mAb1-40 mg/kg, Hu164-67yl−40 mg/kg, Hu164-67yl-20 mg/kg, and Hu147-33wk (40 mg/kg) groups can significantly inhibit Su86.86 tumor growth (p<0.001), and the inhibitory effects on SU86.86 tumors are dose-dependent to a certain extent. In addition, there was no significant change in the body weight of each group of mice.

TABLE 25
The results of the inhibition of SU86.86
mouse xenograft tumors by CTGF antibodies

| | | | | | % | |
|---|---|---|---|---|---|---|
| | | | | | Tumor | |
| | Mean tumor | | Mean tumor | | growth | P (vs |
| | volume (mm³) | | volume (mm³) | | inhibition | blank |
| Group | D 0 | SEM | D 18 | SEM | D 18 | control) |
| Human IgG | 144.35 | 12.30 | 1087.10 | 148.21 | — | |
| mAb1 | 141.18 | 11.72 | 655.75 | 144.46 | 45 | p < 0.001 |
| (40 mg/kg) | | | | | | |
| Hu164-67yl | 138.75 | 8.71 | 582.95 | 76.14 | 53 | p < 0.001 |
| (40 mg/kg) | | | | | | |
| Hu164-67yl | 139.87 | 9.33 | 640.26 | 96.70 | 47 | p < 0.001 |
| (20 mg/kg) | | | | | | |
| Hu147-33wk | 138.00 | 10.11 | 569.66 | 77.40 | 54 | p < 0.001 |
| (40 mg/kg) | | | | | | |

### II. Formulation

### The Equipment, Antibodies, and Methods Used in the Preparation of Formulations and Detection are Shown Below.

### SEC Molecular Exclusion Chromatography:

This is a method for analyzing the separation of a solute by the relative relationship between the pore size of the gel pores and the size of the polymer sample molecule coil.

SEC % (SEC monomer content percentage)=A monomer/A total×100% (A monomer represents the peak area of the main peak monomer in the sample, and A total represents the sum of all peak areas).

Instrument for SEC determination: Agilent 1260; column: waters, XBrige BEH200A SEC (300×7.8 mm 3.5 m).

### NR-CE Capillary Gel Electrophoresis:

This is a method of moving the gel into a capillary as a supporting medium for electrophoresis and separating according to the molecular weight of the sample under a certain voltage.

Non-reduced CE purity percentage=A main peak/A total×100% (A main peak represents the light chain main peak area+the heavy chain main peak area, and A total represents the sum of all peak areas).

Instrument for CE determination: Beckman, model: plus800.

iCIEF Imaged Capillary Isoelectric Focusing Electrophoresis:

This is a technique for separating according to the difference of isoelectric points pI of proteins.

iCIEF neutral peak content percentage=neutral peak area/total area×100% (total area represents the sum of areas of acidic, neutral and basic peaks).

Manufacturer of instrument for the iCIEF determination: simple protein, model: muarice.

### Osmotic Pressure Measurement:

The freezing point method was used for measuring the osmotic pressure. The freezing point of a solution is measured by using a high-sensitivity temperature-sensing element on the basis of the proportional relation between the freezing point depression value and the molar concentration of the solution, and then converted into the osmotic pressure through electric quantity. Manufacturer of instrument: Loser, model: OM815.

### Protein Concentration Measurement:

Instrument for protein concentration measurement: ultraviolet-visible spectrophotometer; model: Nano Drop oneC; optical path length: 1 mm. The following antibody concentrations are calculated based on protein concentrations.

### CTGF Antibodies

The anti-CTGF antibody used in the following examples was Hu164-67yl, wherein the antibody used in samples 1-6 of Example 2-1, Example 2-2, Example 2-3, and Example 2-4 and samples 1 and 2 of Examples 2-5 was obtained by affinity chromatography after protein expression; the antibody used in samples 7 and 8 of Examples 2-4 and samples 3-9 of Examples 2-5 was obtained by further subjecting the aforementioned antibody to ion exchange chromatography and filtration.

### Example 2-1. Formulation Buffer System and pH Value Screening

Liquid formulations comprising 200 mg/mL Hu164-67yl were prepared. The formulations further comprised 0.4 mg/mL polysorbate 80 (PS80), 70 mg/mL sucrose, and different buffer systems. The buffer systems were 50 mM acetic acid-sodium acetate (AA) pH 5.0, 5.5; 50 mM histidine-acetate (His-AA) pH 5.5; 50 mM sodium succinate (SA) pH 5.5; 50 mM sodium citrate (CA) pH 5.5; 50 mM histidine-hydrochloride buffer (His-HCl) pH 5.5, 6.0, 6.5; and 50 mM sodium dihydrogen phosphate-disodium hydrogen phosphate (PB) pH 6.5, 7.0. Each of the formulations was filtered and bottled, and the bottles were stoppered and capped. Then the formulations were left to stand at a constant temperature of 40° C. for 1 month, and the indexes such as SEC and non-reduced CE were evaluated.

The results are shown in Table 26. The SEC data show that in the case of standing at 40° C. for 1 month, the reductions in the main peaks of the samples using AA (pH 5.5), His-AA (pH 5.5), CA (pH 5.5), and His-HCl (pH 5.5) are smaller than those of the other samples. The NR-CE data show that in the case of standing at 40° C. for 1 month, the reduction in the main peak of the sample using His-HCl (pH 5.5) is the smallest (5.1%).

TABLE 26
The results of the pH and buffer system screening

| | | SEC | ΔSEC | NR-CE | ΔNR-CE |
|---|---|---|---|---|---|
| Buffer system | Conditions | % | % | % | % |
| 50 mM AA | D 0 | 97.8 | | 93.3 | |
| pH 5.0 | 40° C. M 1 | 91.5 | 6.3 | 79.9 | 13.4 |
| 50 mM AA | D 0 | 97.8 | | 93.6 | |
| pH 5.5 | 40° C. M 1 | 93.2 | 4.6 | 84.0 | 9.6 |
| 50 mM His-AA | D 0 | 97.9 | | 93.6 | |
| pH 5.5 | 40° C. M 1 | 92.0 | 5.9 | 83.5 | 10.1 |
| 50 mM CA | D 0 | 97.9 | | 94.2 | |
| pH 5.5 | 40° C. M 1 | 92.8 | 5.1 | 82.2 | 12.0 |
| 50 mM SA | D 0 | 97.9 | | 93.4 | |
| pH 5.5 | 40° C. M 1 | 87.3 | 10.6 | 76.9 | 16.5 |
| 50 mM His-HCl | D 0 | 97.9 | | 93.4 | |
| pH 5.5 | 40° C. M 1 | 92.9 | 5.0 | 88.3 | 5.1 |
| 50 mM His-HCl | D 0 | 98.0 | | 93.7 | |
| pH 6.0 | 40° C. M 1 | 91.2 | 6.9 | 88.0 | 5.7 |
| 50 mM His-HCl | D 0 | 98.0 | | 93.4 | |
| pH 6.5 | 40° C. M 1 | 89.8 | 8.2 | 86.3 | 7.1 |
| 50 mM PB | D 0 | 97.9 | | 93.8 | |
| pH 6.5 | 40° C. M 1 | 88.0 | 9.9 | 86.3 | 7.5 |
| 50 mM PB | D 0 | 97.8 | | 93.9 | |
| pH 7.0 | 40° C. M 1 | 84.9 | 12.9 | 82.5 | 11.4 |

Note:
in the table, “D” denotes day; for example, D 7 denotes 7 days, and so on; D 0 denotes the start of the experiment; “M” denotes month; for example, M 1 denotes 1 month, and so on.

### Example 2-2. Long-Term Stability Testing

A formulation comprising 200 mg/mL Hu164-67yl, 50 mM His-HCl (pH 5.5), 70 mg/mL sucrose, and 0.4 mg/mL PS80 was prepared. Samples were left under 25° C. and 4° C. conditions to investigate stability. The results are shown in Table 27.

TABLE 27
Long-term stability testing

| Conditions | SEC % | NR-CE % | iCIEF % |
|---|---|---|---|
| D 0 | 97.9 | 93.4 | 57.1 |
| 4° C. M 3 | 96.7 | 93.7 | 57.6 |
| 4° C. M 5 | 97.4 | 93.4 | 57.5 |
| 25° C. M 3 | 94.0 | 92.0 | 51.4 |
| 25° C. M 5 | 94.3 | 90.3 | 46.0 |

Note:
in the table, “M” denotes month; for example, M 3 denotes 3 months, and so on. The same applies hereinafter.

The results show that: after the samples were left to stand at 25° C. for 5 months, the purity slightly decreased, but the decrease was within an acceptable range; after the samples were left to stand at 4° C. for 5 months, all the test indexes did not change significantly, and accordingly, this formula has better stability.

### Example 2-3. Protein Concentration Screening

Liquid formulations were prepared according to the following formulas:
1) 200 mg/mL Hu164-67yl, 50 mM His-HCl (pH 6.0), 0.4 mg/mL PS80, and 70 mg/mL sucrose
2) 150 mg/mL Hu164-67yl, 50 mM His-HCl (pH 6.0), 0.4 mg/mL PS80, and 70 mg/mL sucrose

The two formulations were filtered and bottled, and the bottles were stoppered and capped. The samples were subjected to 40° C. stability testing and long-term stability testing, with SEC, iCIEF, and non-reduced CE as indexes for evaluation.

TABLE 28
The results of the protein concentration screening

| | | | | NR- | ΔNR- | | |
|---|---|---|---|---|---|---|---|
| | | SEC | ΔSEC | CE | CE | iCIEF | ΔiCIEF |
| Group | Conditions | % | % | % | % | % | % |
| 1 | D 0 | 98.0 | | 93.7 | | 56.9 | |
| | 40° C. M 1 | 91.2 | 6.8 | 88.0 | 5.7 | 35.7 | 21.2 |
| | 25° C. M 1 | 96.7 | 1.3 | 94.1 | | 52.1 | 4.8 |
| | 25° C. M 3 | 93.2 | 4.8 | 93.2 | 0.5 | 43.0 | 13.9 |
| | 25° C. M 5 | 92.3 | 5.7 | 90.2 | 3.5 | 32.8 | 24.1 |
| | 4° C. M 3 | 96.5 | 1.5 | 93.9 | | 56.8 | 0.1 |
| | 4° C. M 5 | 97.0 | 1.0 | 93.0 | 0.7 | 56.9 | 0.0 |
| 2 | D 0 | 98.0 | | 93.3 | | 56.8 | |
| | 40° C. M 1 | 91.7 | 6.3 | 87.6 | 5.7 | 36.0 | 20.8 |
| | 25° C. M 1 | 97.0 | 1.0 | 93.2 | 0.1 | 53.5 | 3.3 |
| | 25° C. M 3 | 93.9 | 4.1 | 93.2 | 0.1 | 50.2 | 6.6 |
| | 25° C. M 5 | 94.1 | 3.9 | 89.6 | 3.7 | 45.2 | 11.6 |
| | 4° C. M 3 | 96.6 | 1.4 | 94.3 | | 56.2 | 0.6 |
| | 4° C. M 5 | 97.3 | 0.7 | 93.1 | 0.2 | 56.3 | 0.5 |

The results show that after samples 1) and 2) were left to stand under 40° C. Ml, 25° C. M3, and 4° C. M5 conditions, there was no significant difference in purity. After standing under 25° C. M5 conditions, the reductions in the SEC and iCIEF main peaks of sample 1) were slightly greater than those of sample 2).

### Examples 2-4. Surfactant Screening

Formulations comprising 150 mg/mL Hu64-67yl, 50 mM His-HCl pH 5.5, 70 mg/mL sucrose, and different surfactants were prepared. Each of the formulations was filtered and bottled, and the bottles were stoppered and capped. Samples were taken and subjected to shaking (25° C., 300 rpm), 5 freeze-thaw cycles (FT5C, conditions: −35° C.-2 to 8° C.), 40° C. high-temperature standing, and long-term stability (4° C., M3) testing, and SEC, non-reduced CE, and iCIEF were examined. The specific formula designs are shown in Table 29.

TABLE 29
The results of the surfactant type and concentration screening

| Group | Buffer system | Conditions | SEC % | NR-CE % | iCIEF % |
|---|---|---|---|---|---|
| 1 | 0.2 mg/mL | D 0 | 97.8 | 93.3 | 56.9 |
| | PS20 | Shaking | 97.4 | 95.1 | 57.1 |
| | | FT5C | 96.8 | 92.2 | 56.8 |
| | | 4° C. M 3 | 97.6 | 93.1 | 58.7 |
| 2 | 0.4 mg/mL | D 0 | 97.8 | 93.4 | 56.6 |
| | PS20 | Shaking | 97.4 | 95.0 | 57.0 |
| | | FT5C | 96.7 | 92.5 | 56.7 |
| | | 4° C. M 3 | 97.5 | 93.4 | 58.7 |
| 3 | 0.6 mg/mL | D 0 | 97.8 | 93.9 | 57.0 |
| | PS20 | Shaking | 97.3 | 94.8 | 57.5 |
| | | FT5C | 96.6 | 92.1 | 54.0 |
| | | 4° C. M 3 | 97.2 | 93.5 | 58.1 |
| 4 | 0.2 mg/mL | D 0 | 97.7 | 93.3 | 56.9 |
| | PS80 | Shaking | 97.3 | 95.1 | 57.1 |
| | | FT5C | 96.6 | 92.1 | 56.6 |
| | | 4° C. M 3 | 97.5 | 93.7 | 58.0 |
| 5 | 0.4 mg/mL | D 0 | 97.7 | 93.8 | 57.2 |
| | PS80 | Shaking | 96.8 | 95.0 | 57.1 |
| | | FT5C | 96.6 | 92.0 | 56.7 |
| | | 4° C. M 3 | 97.5 | 93.7 | 57.7 |
| 6 | 0.6 mg/mL | D 0 | 97.7 | 93.3 | 57.0 |
| | PS80 | Shaking | 97.2 | 94.9 | 57.1 |
| | | FT5C | 96.5 | 92.0 | 56.2 |
| | | 4° C. M 3 | 97.5 | 93.4 | 57.7 |
| 7 | 0.4 mg/mL | D 0 | 98.4 | 95.2 | 61.9 |
| | poloxamer 188 | 40° C. M 1 | 94.9 | 91.1 | 41.5 |
| 8 | 0.4 mg/mL | D 0 | 98.4 | 94.7 | 61.8 |
| | PS80 | 40° C. M 1 | 94.2 | 90.8 | 40.2 |

The experimental results show that there was no significant difference in the purity results of the samples comprising different types and concentrations of polysorbate. After standing at 40° C. for 1 month, there was no significant difference in the purity of two samples comprising the same concentration of PS80 and poloxamer 188.

### Examples 2-5. Formulation Formula Optimization

Liquid formulations were prepared according to the following formulas:
1) 200 mg/mL Hu164-67yl, 50 mM His-HCl (pH 6.0), 70 mg/mL sucrose, and 0.4 mg/mL PS80
2) 200 mg/mL Hu164-67yl, 50 mM His-HCl (pH 6.0), 70 mg/mL trehalose, and 0.4 mg/mL PS80
3) 150 mg/mL Hu164-67yl, 50 mM His-HCl (pH 5.5), 70 mg/mL sucrose, and 0.4 mg/mL PS80
4) 150 mg/mL Hu164-67yl, 50 mM His-HCl (pH 5.5), 70 mg/mL sucrose, 0.4 mg/mL PS80, and 2% PEG 3350
5) 150 mg/mL Hu164-67yl, 50 mM His-HCl (pH 5.5), 70 mg/mL sucrose, 0.4 mg/mL PS80, and 50 mM arginine
6) 150 mg/mL Hu164-67yl, 50 mM His-HCl (pH 5.5), 70 mg/mL sucrose, 0.4 mg/mL PS80, and 1% mannitol
7) 150 mg/mL Hu164-67yl, 50 mM His-HCl (pH 5.5), 70 mg/mL sucrose, 0.4 mg/mL PS80, and 0.5 mM EDTA
8) 150 mg/mL Hu164-67yl, 50 mM His-HCl (pH 5.5), 70 mg/mL sucrose, 0.4 mg/mL PS80, and 5 mM EDTA
9) 150 mg/mL Hu164-67yl, 50 mM His-HCl (pH 5.5), 70 mg/mL sucrose, 0.4 mg/mL PS80, and 10 mM EDTA

Each of the formulations was filtered and bottled, and the bottles were stoppered and capped. Samples were subjected to 40° C. stability testing, with SEC, iCIEF, and non-reduced CE as indexes for evaluation.

TABLE 30
Formula Optimization

| | | | | NR- | ΔNR- | | |
|---|---|---|---|---|---|---|---|
| | | SEC | ΔSEC | CE | CE | iCIEF | ΔiCIEF |
| Group | Conditions | % | % | % | % | % | % |
| 1 | D 0 | 98.0 | | 93.7 | | 56.9 | |
| | 40° C. M 1 | 91.2 | 6.1 | 88.0 | 5.7 | 35.7 | 21.2 |
| 2 | D 0 | 98.0 | | 93.5 | | 56.8 | |
| | 40° C. M 1 | 90.8 | 7.2 | 88.9 | 4.6 | 34.0 | 22.8 |
| 3 | D 0 | 98.4 | | 94.7 | | 61.8 | |
| | 40° C. M 1 | 94.2 | 4.2 | 90.8 | 3.9 | 40.2 | 21.6 |
| 4 | D 0 | 98.3 | | 94.4 | | 61.5 | |
| | 40° C. M 1 | 94.3 | 4.0 | 90.5 | 3.9 | 40.6 | 20.9 |
| 5 | D 0 | 98.4 | | 94.5 | | 61.7 | |
| | 40° C. M 1 | 94.2 | 4.2 | 91.0 | 3.5 | 40.6 | 21.1 |
| 6 | D 0 | 98.0 | | 95.1 | | 60.7 | |
| | 40° C. M 1 | 94.1 | 3.9 | 91.1 | 4.0 | 40.1 | 20.6 |
| 7 | D 0 | 98.3 | | 94.8 | | 61.7 | |
| | 40° C. M 1 | 94.7 | 3.6 | 91.8 | 3.0 | 41.0 | 20.7 |
| 8 | D 0 | 98.3 | | 94.6 | | 61.6 | |
| | 40° C. M 1 | 94.7 | 3.6 | 91.6 | 3.0 | 40.9 | 20.7 |
| 9 | D 0 | 98.3 | | 94.5 | | 62.2 | |
| | 40° C. M 1 | 94.7 | 3.6 | 92.2 | 2.3 | 40.7 | 21.5 |

The results show that: after the samples were left to stand at 40° C. for 1 month, there was no significant difference in purity between the samples in which only sucrose was added and the samples in which other stabilizers were added. The formula stability was good under these conditions. The results suggest that addition of stabilizers other than sucrose will not affect the formula stability, and the formulas that do not comprise other stabilizers have achieved the stability of the formulas that comprise other stabilizers.

1. A pharmaceutical composition, comprising an anti-CTGF antibody and a buffer, wherein the anti-CTGF antibody comprises a heavy chain variable region and a light chain variable region, wherein:
i) the heavy chain variable region comprises HCDR1, HCDR2, and HCDR3 having identical sequences to those of a heavy chain variable region set forth in SEQ ID NO: 8, and the light chain variable region comprises LCDR1, LCDR2, and LCDR3 having identical sequences to those of a light chain variable region set forth in SEQ ID NO: 9;
ii) the heavy chain variable region comprises HCDR1, HCDR2, and HCDR3 having identical sequences to those of a heavy chain variable region set forth in SEQ ID NO: 6, and the light chain variable region comprises LCDR1, LCDR2, and LCDR3 having identical sequences to those of a light chain variable region set forth in SEQ ID NO: 7;
ii-i) the heavy chain variable region comprises HCDR1, HCDR2, and HCDR3 having identical sequences to those of a heavy chain variable region set forth in SEQ ID NO: 69, and the light chain variable region comprises LCDR1, LCDR2, and LCDR3 having identical sequences to those of a light chain variable region set forth in SEQ ID NO: 70; or
ii-ii) the heavy chain variable region comprises HCDR1, HCDR2, and HCDR3 having identical sequences to those of a heavy chain variable region set forth in SEQ ID NO: 85, and the light chain variable region comprises LCDR1, LCDR2, and LCDR3 having identical sequences to those of a light chain variable region set forth in SEQ ID NO: 70;
the buffer is a histidine salt buffer.

2. The pharmaceutical composition according to claim 1, wherein the anti-CTGF antibody comprises a heavy chain variable region and a light chain variable region, wherein:
the heavy chain variable region comprises HCDR1, HCDR2, and HCDR3 set forth in SEQ ID NO: 16, SEQ ID NO: 17, and SEQ ID NO: 18, respectively, and the light chain variable region comprises LCDR1, LCDR2, and LCDR3 set forth in SEQ ID NO: 19, SEQ ID NO: 20, and SEQ ID NO: 21r.

3. The pharmaceutical composition according to claim 1, wherein the pharmaceutical composition has a pH of about 5.0 to about 6.5.

4. The pharmaceutical composition according to claim 1, wherein the anti-CTGF antibody is at a concentration of about 100 mg/mL to about 200 mg/mL.

5. The pharmaceutical composition according to claim 1, wherein the pharmaceutical composition further comprises a surfactant, and the surfactant is a polysorbate or poloxamer.

6. The pharmaceutical composition according to claim 5, wherein the surfactant is at a concentration of about 0.05 mg/mL to about 1.0 mg/mL.

7. The pharmaceutical composition according to claim 1, wherein the pharmaceutical composition further comprises a sugar, and the sugar is selected from one or more of sucrose, mannitol, and trehalose.

8. The pharmaceutical composition according to claim 7, wherein the sugar is at a concentration of about 20 mg/mL to about 100 mg/mL.

9. The pharmaceutical composition according to claim 1, wherein the buffer is at a concentration of about 5 mM to about 100 mM.

10. The pharmaceutical composition according to claim 1, wherein the pharmaceutical composition further comprises a stabilizer, and the stabilizer is selected from the group consisting of PEG, arginine, and EDTA.

11. The pharmaceutical composition according to claim 1, comprising the following components:
(a) the anti-CTGF antibody at about 150 mg/mL to about 200 mg/mL, (b) about 0.2 mg/mL to about 0.6 mg/mL polysorbate, (c) about 40 mg/mL to about 80 mg/mL sugar, and (d) about 30 mM to about 70 mM histidine salt buffer; the pharmaceutical composition having a pH of about 5.0 to about 6.0.

12. A lyophilized formulation, wherein the lyophilized formulation can, upon reconstitution, form the pharmaceutical composition according to claim 1.

13. A method for preparing a lyophilized formulation, comprising a step of lyophilizing the pharmaceutical composition according to claim 1.

14. A lyophilized formulation, wherein the formulation is obtained by lyophilizing the pharmaceutical composition according to claim 1.

15. A reconstituted solution, wherein the reconstituted solution is prepared by reconstituting the lyophilized formulation according to claim 12.

16. The pharmaceutical composition according to claim 1 wherein the composing is formulated for intravenous, subcutaneous, intraperitoneal, or intramuscular injection.

17. An article of manufacture, comprising a container, wherein the container contains the pharmaceutical composition according to claim 1.

18. A method for treating or preventing a CTGF-associated disease, wherein the method comprises administering to a subject an effective amount of the pharmaceutical composition according to claim 1,
wherein the CTGF-associated disease is a fibrotic disease, hypertension, diabetes, myocardial infarction, arthritis, a CTGF-associated disease of cellular proliferation, atherosclerosis, glaucoma, or cancer.

19. The pharmaceutical composition according to claim 1, wherein the heavy chain variable region has an amino acid sequence set forth in SEQ ID NO: 34, and the light chain variable region has an amino acid sequence set forth in SEQ ID NO: 30.

20. The pharmaceutical composition according to claim 1, wherein the anti-CTGF antibody comprises a heavy chain set forth in SEQ ID NO: 61 and a light chain set forth in SEQ ID NO: 64.

21. The pharmaceutical composition according to claim 1, comprising the following components:
(a) the anti-CTGF antibody at about 150 mg/mL,
(b) about 0.4 mg/mL polysorbate 80,
(c) about 70 mg/mL sucrose, and
(d) about 50 mM histidine-hydrochloride buffer;
the pharmaceutical composition having a pH of about 5.5 to about 5.7; the anti-CTGF antibody comprising a heavy chain set forth in SEQ ID NO: 61 and a light chain set forth in SEQ ID NO: 64.

22. The method according to claim 18 wherein the fibrotic disease is selected from the group consisting of idiopathic pulmonary fibrosis, diabetic nephropathy, diabetic retinopathy, osteoarthritis, scleroderma, chronic heart failure, liver cirrhosis, and renal fibrosis; and
the cancer is selected from the group consisting of acute lymphoblastic leukemia, dermatofibroma, breast cancer, angiolipoma, angioleiomyoma, desmoplastic cancer, prostate cancer, ovarian cancer, colorectal cancer, pancreatic cancer, gastrointestinal cancer, and liver cancer.
